(19) United States
(12) Patent Application Publication
(10) Pub. No.: US 2010/0292200 A1
(43) Pub. Date: Nov. 18, 2010

(54) Methods for Modulating Apoptosis in Platelets

(75) Inventors: Thomas Benjamin Kile, Victoria (AU); David C.S. Huang, Victoria (AU); Kylie D. Mason, Victoria (AU); Andrew Warwick Roberts, Victoria (AU); Marina Rose Carpinelli, Victoria (AU)

Correspondence Address: CLARK & ELBING LLP, 101 FEDERAL STREET, BOSTON, MA, 02110, US

(73) Assignee: The Walter and Eliza Hall Institute of Medical Research, Parkville, Victoria (AU)

(21) Appl. No.: 12/377,088

(22) PCT Filed: Aug. 10, 2007
(86) PCT No.: PCT/AU07/01121

§ 371 Date: Apr. 3, 2009

Related U.S. Application Data
(60) Provisional application No. 60/919,264, filed on Mar. 20, 2007.

(30) Foreign Application Priority Data
Aug. 11, 2006 (AU) 2006904386
Mar. 20, 2007 (AU) 2007901452

(51) Int. Cl. A61K 31/573; A01N 1/02; C12N 5/078; G01N 33/53; A61P 7/00

(52) U.S. Cl. 514/180; 435/2; 514/179; 435/375; 435/7.1; 435/372

(57) ABSTRACT

The description discloses methods of enhancing or maintaining the viability or lifespan of platelets comprising administering an agent that down modulates apoptosis. The description also discloses a method of decreasing the survival, lifespan or viability of platelets comprising administering an effective amount of an agent that enhances apoptosis.

### FIELD

The present invention relates to the practical application of information concerning the regulation of apoptosis to the field of platelet regulation. In particular, the invention relates to Bcl-2 family members, their regulators as well as more broadly to pharmacological agents that effectively modulate apoptosis. The present invention provides new targets, methods and agents for use in modulating interactions involving platelets or their precursors. In particular, the invention contemplates methods and agents when used for the treatment or prevention of conditions associated with inherited or acquired thrombocytoses or thrombocytopaenias, such, as, without limitation, vascular disease and bleeding disorders. The invention further relates to methods and agents for preparing and storing blood and blood derivatives and to methods of modulating, inter alia, platelet turnover, hemostasis, clot formation, tissue remodelling and healing.

### BACKGROUND

Bibliographic details of publications referred to by author in this specification are collected at the end of the description.

Reference in this specification to any prior publication (or information derived from it), or to any matter which is known, is not, and should not be taken as an acknowledgment or any form of suggestion that the prior publication (or information derived from it) or known matter forms part of the common general knowledge in the field of endeavour to which this specification relates.

Platelets are small, anuclear fragments of megakaryocytes that circulate in the blood and make essential contributions to functions such as blood clotting and wound healing. They are produced by megakaryocytes: large, polyploid cells that develop in the bone marrow and spleen. Megakaryocytes shed platelets into the blood stream where, in humans, they circulate for around 10 days (Leeksma et al., Nature, 175:552-553, 1955) before being destroyed by the reticuloendothelial system, primarily in the liver and spleen. Like all lineages of blood cells, the steady state number of mature platelets is the result of a balance between their production and destruction. In normal individuals, precise control of proliferation, differentiation, survival and clearance of these cells ensures maintenance of homeostasis, and reduces the likelihood of haemorrhage should platelet counts fall or thrombosis resulting from excess platelet production.

If this delicate balance is perturbed, thrombocytopaenia, or low platelet count, can ensue. Thrombocytopaenia is a common problem in the clinic, particularly in haemtological and oncological practice, as it leads to potentially fatal hemorrhagic episodes. It can occur congenitally, with a number of inherited disorders having been defined (Drachman, Blood, 103:390-398, 2004), but the majority of thrombocytopaenias seen in the clinic are the result of other causes. It can be a major problem for patients undergoing cancer chemotherapy. Acute episodes of cytotoxic drug-related thrombocytopaenia, in addition to putting the patient at immediate risk, can force dose modifications or treatment withdrawal, thus blunting treatment efficacy. Thrombocytopaenia is also frequently encountered in myelodysplasia syndromes (MDS), idiopathic thrombocytopaenia purpura (ITP) and chronic liver disease, and is associated with viral infections, particularly AIDS (Kuter et al., Blood, 100:3457-3469, 2002). In these more chronic contexts, thrombocytopaenia may result from defective platelet production or elevated platelet destruction, often as the result of autoimmune reactions. Treatment for low platelet numbers includes platelet transfusion and, potentially, administration of thrombopoietin.

Platelet mediated thrombosis is a major mechanism leading to vascular diseases such as cardiovascular, cerebrovascular and peripheral vascular diseases. Control of platelet levels or activity is an essential component of anti-thrombosis treatments. Anti-platelet agents such as aspirin, non-steroidal anti-inflammatory agents, β-lactam antibiotics, quinidine, calcium channel blockers, ticlopidine, clopidogrel and reopro are used in treating myocardial infarction and ischemic stroke or their subsequent complications, however, more effective and safer drugs are needed. Pro-thrombotic states are seen in subjects with conditions such as myeloproliferative disorders, chronic pulmonary obstructive disease and essential thrombocytosis.

The molecular regulation of apoptosis has been characterised in considerable detail over the past 20 years (Marsden et al., Annu. Rev. Immunol., 27:71-105, 2003). Apoptosis is executed by a family of aspartate-specific cysteine proteases (caspases). Many caspases exist in an inactive form in healthy cells, and are activated in response to two major signalling pathways that induce apoptosis. The first pathway is induced by developmental cues, cytokine withdrawal and other stress stimuli, and is regulated by the Bcl-2 protein family, which includes both pro-apoptotic members (e.g. Bax, Hrk, Bim) and pro-survival members (e.g. Bcl-2, Bcl-x_{L}). The second apoptotic pathway involves ligand binding to death receptors (e.g. Fas), causing formation of a death-inducing signalling complex.

The Bcl-2 family of proteins plays a central role in regulating developmentally programmed and stress induced cell deaths (Adams, Genes Dev., 17:2481-2495, 2003; Danial et al., Cell, 116:205-219, 2004). One sub-class of the family, most closely related to Bcl-2 and including Bcl-x_{L} (Boise et al., Cell, 74:597-608, 1993), Bcl-w, Mcl-1 and A1 promote survival of particular cells. They maintain cell survival until their activity is neutralised by direct binding of the distantly related pro-apoptotic BH3-only proteins such as Bim, Bad or Bid (Huang et al, Cell, 103:839-842, 2000). The precise biochemical action of the pro-survival proteins such as Bcl-x_{L} is controversial (Adams, 2003 (supra); Danial et al., 2004 (supra); Willis and Adams, Curr. Opin. Cell. Biol., 17:617-625, 2005), although it is likely that they control the action of a second class of pro-apoptotic family members, the multi-domain proteins Bax and Bak. These play an essential role in mediating apoptosis (Cheng et al., Mol. Cell., 8:705-711, 2001; Lindsten et al., Mol. Cell., 6:1389-1399, 2000; Rathmell et al., Nature Immunology, 3:932-939, 2002; Zong et al., Genes Dev., 15:1481-1486, 2001) probably by damaging intracellular membranes such as the outer mitochondrial membrane, thereby precipitating the release of pro-apoptogenic factors such as cytochrome c normally sequestered within the organelles into the cytoplasm to promote caspase activation (Green et al., Science, 305:626-629, 2004; Green et al., Science, 281:1309-1311, 1998; Yang et al., Science, 275:1129-1132, 1997).

Our understanding of the molecular control of platelet, numbers is incomplete and many cases of inherited thrombocytopaenias remain unexplained (Drachman, 2004 (supra)). It has been assumed that the major physiological controls on circulating platelet numbers are platelet production and platelet destruction by the reticuloendothelial system. In this scenario, the survival of platelets in the circulation has been thought to be a function of the metabolic capacity that a platelet acquires from its precursor, the megakaryocyte, and the cell death machinery has been conventionally thought to play little or no role in modulating platelet levels and activity.

### SUMMARY

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of sequence identifiers is provided in Table 1. A sequence listing is provided after the claims.

Genes and other genetic material (e.g. mRNA, constructs etc) are represented in italics and their proteinaceous expression products are represented in non-italicised form. Thus, Bcl-x_{L} is an expression product of Bcl-x. The term “Bcl-x_{L}” or “Bcl-x” or “Bak” or “Bak” or “Bax” or “Bax” is used to encompass all functionally analogous homologs in any species.

A genetic screen for mutations that cause thrombocytopaenia in mice has led to the identification of pro-survival Bcl-x_{L} as the key regulator of platelet survival. When Bcl-x_{L} is functionally compromised, either genetically or chemically, the levels of circulating platelets drop, due to reductions in their life span. Mouse strains that harbour mutated alleles of Bcl-x are thrombocytopenic and in accord with the hypothesis that Bcl-x_{L} is the main survival factor controlling platelet survival, mice that have their Bcl-x gene specifically targeted are similarly affected. Thus, the control of platelet survival has an important role in controlling the numbers of circulating platelets which is distinct from the regulation of megakaryocyte differentiation and platelet production.

As disclosed herein, Bcl-x_{L} maintains the survival of platelets, and if Bcl-x_{L} activity is compromised, platelet life span and consequently the total number of platelets in the circulation is reduced. Deletion of the downstream pro-apoptotic cell death mediators controlled by Bcl-x_{L} reverses the thrombocytopaenia induced by loss of Bcl-x_{L}. In particular, removal of Bak reverses the loss in platelet numbers cause by depleting Bcl-x_{L}, thus demonstrating that the balance between pro-survival Bcl-x_{L} and pro-apoptotic Bak is the major determinant of platelet survival in vivo. Bak^{-/-} platelets have an increased half-life in vivo, that is they are cleared more quickly from the circulation. Specifically, the loss of Bak increased platelet t_{1/2} by approximately 40% from 47 hrs to 67 hrs (FIG. 6C). The removal of the Bak-related molecule, Bax also has the effect of enhancing platelet life span, but to a lesser extent.

The present invention provides, therefore, methods of modulating the number and/or survival of platelets comprising administering an effective amount of an agent that modulates apoptosis. In some embodiments, the methods are in vitro methods. In other embodiments, the methods are in vivo or ex vivo. A large number of agents including cytokines and pharmacological agents such as antisense molecules or small peptide or non-peptide inhibitors or binding molecules are known to the skilled artisan that agonise or antagonise the molecules in the pathway that effects apoptosis in a range of cell types other than platelets.

In another form, the present invention provides for the use of apoptosis modulators in the treatment or prevention of conditions associated with subnormal or supernormal levels of platelets. In another embodiment, the invention provides for the use of these agents in the preparation of a medicament for the treatment or prophylaxis of thrombocytopaenia or thrombocytosis. In another embodiment, the present invention provides agents for use in the treatment of conditions characterised by abnormal or supernormal levels of platelets. In some embodiments, the agent (molecule, compound etc) modulates the functional ability or activity of platelets, such as their ability to aggregate, secrete dense granules stores, express surface markers and adhere to plasma molecules such as fibrinogen or von Willebrand factor.

Thus, for example, the present invention provides agents that promote apoptosis for use in the treatment of pro-thrombotic states. In another example, the invention provides agents that down regulate apoptosis for use in the treatment of thrombocytopaenia. Thus, for examples, caspase inhibitors are administered. In another example, agents that promote Bcl-x_{L} level or activity in platelets or their precursors (megakaryocytes) are contemplated.

In accordance with one embodiment of the present invention, the apoptosis-modulating agents effectively target or modulate the activity of pro-survival and/or pro-apoptotic members of the Bcl-2 polypeptide family. In some embodiments, the agents modulate Bcl-2 apoptosis pathways and particularly the Bcl-x_{L}/Bak and/or Bcl-x_{L}/Bax mediated apoptosis pathway. In an exemplified embodiment, the agent effectively modulates the activity of Bcl-x_{L} and/or Bak and/or Bax. Specifically, by increasing the activity of pro-survival compared to pro-apoptotic molecules platelet numbers or survival is enhanced. For example, as shown in Example 5, loss of pro-apoptotic Bak ameliorates thrombocytopaenia in a mammalian subject. Similarly, a BH3-domain mimic ABT-737 causes Bak mediated and caspase dependent killing of platelets that is prevented in the absence of Bak or in the presence of a caspase inhibitor (see Example 7). Antagonists of Bax also prolong viability. This is shown, for example, in Example 7, where the effects of a pro-apoptotic agent in mice for deficient Bak and/or Bax were examined. In the absence of Bak, the loss of one Bax allele rendered platelets entirely refractory to ABT-737.

Genes and other genetic material (e.g. mRNA, constructs etc) are represented in italics and their proteinaceous products are represented in non-italicised form. Thus, Bak polypeptide is the product of the Bak gene. The italicised or non-italicised forms are used to encompass homologsand functional variants in any animal and preferably mammalian species. In some preferred embodiments, the invention is directed to a human homolog of Bcl-2 family members.

In some embodiments, the subject methods are useful, for example, in the treatment of conditions associated with thrombocytopaenia and for enhancing the viability of platelets in blood derivative products. In relation to thrombocytopaenia, in some embodiments, this is inherited thrombocytopaenia. In other embodiments, thrombocytopaenia is acquired. Accordingly, methods are considered for enhancing or maintaining the viability or lifespan of platelets comprising administering an effective amount of an agent that down modulates apoptosis. In an illustrative embodiment, the anti-apoptotic agent is an agent identified in the herein disclosed cellular screen. In an illustrative embodiment, the agent is selected from one of the corticosteroid molecules set out in FIG. 10 or comprises the general structure set out in FIG. 10. As described herein, these agents strongly inhibited killing in mammalian cells exposed to an apoptosis inducing amount of a Bcl-x_{L} antagonist. In some other embodiments, the agent enhances the ratio of Bcl-x_{L}:Bak in a cell. In other embodiments, the agent is an agonist of Bcl-x_{L} mediated apoptosis pathway or an agonist of Bcl-x_{L}. In an especial embodiment, the agent is an antagonist of Bak, or Bax, or Bak and Bax or an antagonist, of downstream effectors of Bak, or Bax, or Bak and Bax activity. In other embodiments, the agent inhibits the uptake or cellular activity of apoptosis inducing agents. In some embodiments the agent (agonist or antagonist) is a small molecule, inhibitory RNA, antibody, aptamer, peptide, foldamer, peptidomimetic including a cyclic peptidomimetic, or a constrained peptide. In accordance with the present invention, molecules identified as anti-apoptotic i.e., those agents which enhance the survival, viability, half-life or life-span of mammalian cells in the herein described cellular screens are useful for enhance the survival, viability, half-life or life-span of a mammalian cell not limited to but including platelets.

The above methods encompass ex vivo administration such as wherein the agent is administered to a blood product containing platelets, such as whole blood or a platelet preparation. The above methods also encompass administration in vivo. In terms of administration, in some embodiments the agent is administered to a subject suffering from or at risk of developing thrombocytopaenia. In an illustrative example, the subject is one receiving any form of chemotherapy such as cytotoxic drugs including antibodies or antigen-binding molecules. One measure of platelet viability or survival is the number or half-life of platelets in circulation, another is their age profile i.e., average age. Other indicia of platelet viability in vitro or in vivo are described in Example 12. In some embodiments of the method, the half-life platelet is enhanced. In other embodiments, the platelets are stored ex vivo. In an illustrative embodiment, the half-life is enhanced by about 40%.

Thus, the present specification describes a method of treating or preventing thrombocytopaenia in a subject comprising identifying a subject suffering from or at risk for thrombocytopaenia; and administering to the identified subject an agent that down modulates apoptosis of platelets. As mentioned above, the agent in some embodiments, enhances the ratio of Bcl-x_{L}:Bak in a platelet. In some embodiments, the agent is an agonist of Bcl-x_{L} mediated apoptosis pathway or an agonist of Bcl-x_{L}. As Bak and Bax are more stable in platlets than Bcl-x, an especial embodiment considers administration of an antagonist of Bak or Bax or Bak and Bax. In some embodiments, the agent is a Bak-binding portion of Bcl-x_{L} or a variant or mimic thereof or a Bax-binding portion of Bcl-x_{L}, or a variant or mimic thereof or a Bak and Bax-binding portion of Bcl-x_{L} or a variant or mimic thereof. In other embodiments, the agent is a gene silencing agent. Alternatively, the agent is an antagonist of downstream effectors of Bak, or Bax, or Bak and Bax activity or an agent that inhibits the uptake or cellular activity of apoptosis inducing agents in platelets. In some embodiments, the agent is an apoptogenic factor inhibitor. In relation to some embodiments the agent (agonist or antagonist) is a small molecule, inhibitory RNA, antibody, aptamer, peptide, peptidomimetic or constrained peptide.

In another aspect, by decreasing the activity of pro-survival in favour of pro-apoptotic molecules, platelet number and/or survival is decreased. In an exemplified embodiment, Bcl-x_{L} is inhibited or antagonised with a Bcl-2 homology domain mimetic agent such as a BH3 homology domain mimetic agent and platelet numbers and/or survival are decreased. In another embodiment, enhancing or agonising Bak activity, directly or indirectly, leads to increased platelet apoptosis. Down regulation of platelet numbers is useful, for example, in the treatment or prevention of conditions associated with thrombosis. Accordingly, in a broad embodiment of this aspect a method is contemplated for decreasing the survival, lifespan, half-life or viability of platelets comprising administering an effective amount of an agent that enhances apoptosis. In some embodiments, the agent is an antagonist of Bcl-x_{L} mediated apoptosis pathway including an antagonist of Bcl-x_{L} polypeptide activity. In other embodiments, the agent is an agonist of Bak polypeptide activity or an agonist of Bax polypeptide activity or an agonist of both Bak and Bax polypeptide activity. In some embodiments the agent of the present invention operates in the Bcl-x_{L} pathway between Bak and/or Bax and caspase activity, here the agent is an agonist of downstream effectors of Bak, or Bax, or Bak and Bax activity. In other embodiments, the agent is an IAP (inhibitor or apoptosis) antagonist, In still other embodiments, the antagonist is a BH3-domain mimic or gene silencing agent or small molecule. In other embodiments the agent (agonist or antagonist) is a small molecule, inhibitory RNA, antibody, aptamer, peptide, peptidomimetic or constrained peptide.

The methods for decreasing the survival, lifespan, half-life or viability of platelets encompass where the agent is administered in vivo or ex vivo. In some embodiments in relation to in vivo or ex vivo administration, the agent administered to a subject tested for thrombocytosis prior to administration. In another aspect, because older platelets are more vulnerable than younger platelets to Bcl-x_{L} antagonists, Bcl-x_{L} antagonists are administered to blood or platelet donors prior to blood or platelet donation for reducing the average age of the platelets in the donated blood or platelets.

For in vivo applications, the above-described agents are administered to a subject in need thereof for treating or preventing thrombocytosis. Thus, a method of treating or preventing thrombocytosis in a subject is considered comprising identifying a subject suffering from or at risk for thrombocytosis; and administering to the identified subject an agent that promotes apoptosis of platelets. In some embodiments, the agent is an antagonist of Bcl-x_{L} mediated apoptosis pathway such as an antagonist of Bcl-x_{L}. In another embodiment, the agent is air agonist of Bak, or Bax, or Bak and Bax, or an agonist of a downstream effector of Bak, or Bax, or Bak and Bax activity. In some embodiments, the antagonist of Bcl-x_{L} mediated apoptosis pathway is a BH3-domain mimic. In another embodiment, the agent is a small molecule, inhibitory RNA, antibody, aptamer, peptide, peptidomimetic, foldamer or constrained peptide. In light of the enhanced vulnerability of platelets to apoptotic modulators, the subject agents may be administered in an amount or for a time which affects platelets but not other mammalian cells, thereby usefully discriminating between target cells. In one embodiment therefore the agent is administered in an amount and/or for a time effective to induce anuclear platelet apoptosis but substantially not apoptosis in nucleated cells.

In another embodiment, the agent modulates the activity of other components in the pathway culminating in programmed cell death. For example, agents that down regulate caspase activity or potentiate IAP activity are useful in promoting platelet viability. Suitable agents are known to those of skill in the art or are identified by the present screening methods or modifications thereof. In another embodiment, down stream effectors include cytochrome C, Apaf-1 and caspases.

Although the present invention has been exemplified using Bcl-x_{L}, Bak or Bax, the present invention is not so limited and extends to all functional homologs, functional isoforms or functional variants, including fragments of Bcl-x_{L}, Bak or Bax. In some embodiments, prosurvival and/or proapoptotic members of the Bcl-2 polypeptide family are regulated. Proapoptotic regulators include Bak, Bok (Mtd), Bax, Bad, Bid, Bik (Blk), Hrk (DP5), BNIP3, Bim, Puma, Noxa, Mule (Lasu/ARF-BPI) and Bmf. Prosurvival members include Bcl-2, Mcl-1, Bcl-w, Bcl-x_{L}, Bcl-B and A1 (Bfl-1 in humans). In other embodiments, one or more Bcl-2 family members are targeted. In some embodiments, agents modulate two or more Bcl-2 family members. In other embodiments, one or more separate agents are co-administered for enhanced efficiency. Preferred agents decrease apoptosis and antagonise Bax and/or Bak or molecules downstream of Bax and/or Bak in the apoptosis pathway.

Reference to co-administration includes simultaneous, sequential and/or spaced administration of two or more agents.

In a related embodiment, the present invention contemplates the use of compositions comprising Bcl-x_{L} or Bak and/or Bax polypeptides or analogs thereof or variants of Bcl-x_{L} or Bak and/or Bax polypeptide or agents that modulate the level or activity of Bcl-x_{L} and/or Bak and/or Bax to up-regulate or down-regulate platelet levels in a subject or in vitro. In one particular embodiment, agents that modulate the level or activity of Bcl-x_{L} or Bak and/or Bax comprise nucleic acid molecules from which Bcl-x_{L} or Bak or Bax polypeptides or peptides are producible.

In another embodiment, the present invention provides agents that modulate apoptosis for use in the treatment and/or prophylaxis of conditions associated with thrombocytopaenia or thrombocytoses. The agents are conveniently in a composition comprising the agent and one or more pharmaceutically acceptable earners, diluents and/or excipients. The agents may also be used in conjunction with further modulators of apoptosis such as caspase inhibitors or modulators of platelet level or activity, such as aspirin. Consequently, the present invention provides compositions or two- or multi-part pharmaceutical compositions comprising in one embodiment at least one modulator of platelet apoptosis and one inhibitor of platelet function. In another embodiment, platelet-protective anti-apoptosis agents are administered as an adjunct to the use of an apoptosis stimulator, for example, in cancer treatment regimes.

In another aspect, the present invention provides methods of screening or testing for agents useful in modulating platelet levels or life span in vitro or in vivo. In some embodiments, agents are tested for their ability to increase or decrease platelet survival, life-span, viability or half life. In some embodiments, agents are tested for their ability to modulate the activity of the Bcl-2 family targets identified herein or molecules downstream of Bcl-2 family members in the pathway leading to apoptosis. In a preferred embodiment, agents which down modulate apoptosis by decreasing the level or activity of Bak and/or Bax or of molecules downstream in the Bax or Bak mediated apoptosis pathway are selected.

Modified non-human animals and isolated cells comprising a partial loss of function mutation in one or more Bcl-2 family genes are also provided. The invention extends to methods of generating further mouse strains comprising crossing the herein described Bcl-x_{L} mutant mice with mice of a different strain in order to produce further mutants for testing.

The invention also provides methods of screening or testing subjects for mutations in Bcl-2 family genes such as Bcl-x, Bak and Bax genes or their genetic or proteinaoeous regulatory molecules, indicative of a particular genetic basis for thrombocytopaenia or thrombocytoses in the subject. Further methods involve measuring the ratio of pro-survival to pro-apoptotic molecules in a subject. Any agent that affects the targets identified in the present invention may be employed to modulate platelet survival, viability or half-life. The present methods include a method of screening for an agent which modulates the survival, lifespan or viability of platelets, said method comprising:
(i) contacting the agent with a system comprising a target selected from the group consisting of a Bcl-x_{L} and/or Bak or Bax polypeptide, and a Bcl-x, Bak or Bax genetic sequence; and
(ii) determining the presence of a complex between the agent and the target, a change in activity of the target, or a change in the level of activity of an indicator of the activity of the target.

In some embodiments, the method comprises screening for a molecule which enhances the survival, lifespan or viability of platelets and/or other mammalian cells. In an illustrative embodiment, the method comprises: (i) combining the molecule with a cell; (ii) contacting the cell with one or more agents that antagonise pro-survival Bcl-2 family molecules in the cell and induce/s apoptosis; (iii) determining the change in survival (viability, lifespan, half-life) of cells in the presence of the molecule relative to a control; and (iv) selecting a molecule which enhances cell survival (viability, half-life). In some embodiments, the method further comprises combining the selected molecule from (iv) with platelets to determine the change in cell survival (viability, half-life) of platelets in the presence of the molecule relative to controls.

To facilitate screening, in some embodiments, the cell is modified to enhance its sensitivity to an apoptosis inducing agent, such as by reducing the level or activity of one or more pro-survival Bcl-2 family members. In other embodiments, the cell is modified to lack one or more pro-survival Bcl-2 family members by gene disruption. In an illustrative embodiment, the cell is an Mcl-1 deficient cell from a multicellular organism and the agent is a Bcl-x_{L} antagonist. In still other embodiments, the method comprises identifying modulation of a Bcl-2 family protein in the cell. The agents and compositions of the present invention include, for example, small or large organic or inorganic chemical molecules, peptides, polypeptides, modified peptides such as constrained peptides, foldamers, peptidomimetics, cyclic peptidomimetics, proteins, lipids, carbohydrates or nucleic acid molecules including antisense or other gene silencing molecules. Small molecules generally have a molecular mass of less than 500 Daltons. Large molecules generally include whole polypeptides or other compounds having a molecular mass greater than 500 Daltons. Agents may comprise naturally occurring molecules, variants (including analogs) thereof as defined herein or non-naturally occurring molecules. Other compositions include cellular, tissue or organ compositions. In particular, the specification considers a modified population of platelets for administration to a subject in need thereof, the platelets comprising a population of platelets stored ex vivo and contacted with an apoptosis antagonist agent to increase platelet half-life. In some embodiments, the agent comprises an agonist of Bcl-x_{L} or an antagonist of Bak.

The specification further considers an apoptosis inhibitor agent for use in the treatment or prevention of thrombocytopaenia. The agents are considered for use by themselves or in conjunction with other treatments. One other treatment is treatment of cancer. In an especial embodiment, the apoptosis inhibitor agent increases the ratio of Bcl-x_{L}:Bak in a platelet for use in the treatment or prevention of thrombocytopaenia. In other embodiments, the apoptosis inhibitor agent is for use in increasing the half-life of stored platelets. Alternatively, the apoptosis promoting agent is for use in reducing the average age of stored platelets wherein a blood or platelet donor is treated with the apoptosis promoting agent prior to giving blood, or for use in the treatment or prevention of thrombocytosis.

Kits are further considered comprising an apoptosis inhibitor agent for use in the treatment or prevention of thrombocytopaenia or for use in increasing the viability or half-life of stored platelets.

The specification further contemplates a composition for the treatment or prevention of thrombocytopaenia comprising an effective amount of an agent capable of inhibiting or delaying or down modulating apoptosis in platelets. In some embodiments, the agent increases the ratio of Bcl-x_{L}:Bak in a platelet. In other embodiments, a composition is considered for increasing the half-life of stored platelets comprising an effective amount of an agent capable of inhibiting apoptosis in the stored platelets. In other embodiments, a composition is considered for the treatment or prevention of thrombocytosis comprising an effective amount of an agent capable of promoting apoptosis in platelets.

The above summary is not and should not be seen in any way as an exhaustive recitation of all embodiments of the present invention.

### BRIEF DESCRIPTION OF THE FIGURES

Some figures contain color representations or entities. Colored versions of the figures are available from the Patentee upon request or from an appropriate Patent Office. A fee may be imposed if obtained from a Patent Office.

FIG. 1 provides a representation of data showing the isolation and molecular identification of mutations in Bcl-x. (A) peripheral blood platelet counts at 7 weeks of age from 810 G₁ offspring of ENU-mutagenized BALB/c males. Each circle represents an individual mouse. Founder animals for the Plt20 and Plt16 pedigrees are indicated. The heritability of three additional thrombocytopaenias (Plt17, Plt18 and Plt21) was confirmed; these pedigrees are at various stages of the genetic mapping process. Plt17 was mapped to chromosome 11 and a mutation in the gene encoding GpIbα identified. Plt18 maps to an interval on chromosome 16, while Plt21 is yet to be assigned a map location. No heritable mutations causing thrombocytosis were identified. (B, C) Mapping haplotypes for Bcl-x^{Plt20} (B) and Bcl-x^{Plt16} (C) respectively. Markers used and their positions on the April 2006 UCSC mouse genome are indicated. Defining recombinant events are shaded gray; those confirmed by heritability testing are shown in bold. An interval of 16.3 Mb was defined for Bcl-x^{Plt20}, between JCCA19 and D2Mit500. The candidate interval for Bcl-x^{Plt16} was refined to 1.9 Mb, between JCCA9 and D2Mit139. (D, E) DNA sequence electropherograms showing the nucleotide changes in animals heterozygous for the Bcl-x^{Plt20} (D) or Bcl-x^{Plt16} (E) mutations. Further sequencing established that neither the Plt16 nor Plt20 mutation is present in the parental BALB/c strain or the C57BL/6 mapping strain. Tyrosine 15 and isoleucine 182, the residues substituted in the Plt20 and Plt16 pedigrees, respectively, are conserved between mouse and human Bcl-x_{L}.

FIG. 2 provides a graphical representation of data showing that like Bcl-x^{Plt20} and Bcl-x^{Plt16} mutant mice, mice lacking one Bcl-x allele are thrombocytopenic. (A) Automated analysis of platelet counts in wild type C57BL/6, Bcl-x^{+/-}, Bcl-2^{+/-}, Bcl-w^{-/-} or Mcl-I^{+/-} male mice. Deletion of one Bcl-x allele caused a significant decrease in platelet number. Results were compared using two-tailed unpaired Student's t-test. *p<0.05. (B) Decreased life span of Bcl-x^{Plt20} platelets. Peripheral blood samples were taken from Bcl-x^{Plt20} mice 0, 4, 8, 12, 24, 28, 48, 72 and 96 h after injection with biotin N-hydroxysuccinimide. Whereas wild type (Bcl-x^{+/+}) platelets exhibited a t_{1/2} of 57 h, consistent with published observations (Berger et al., Blood 92:4446-4452, 1998), the Bcl-x^{Plt20} mutation caused a dose-dependent decrease to ˜24 h in heterozygotes and to ˜10 h in Bcl-x^{Plt20/Plt20} homozygous mice. (C) Bcl-x mutations shorten platelet life spans. Half-lives of platelets in mice of the indicated genotypes determined as in (B). Like the Bcl-x^{Plt20} mutation, Bcl-x^{Plt16} or Bcl-x deletion (Bcl-x^{+/-}) also decreased platelet half-lives relative to that of wild type (Bcl-x^{+/+}) littermate controls, ^{†}Detailed information on the genetic background of the mice is provided in Experimental Procedures. (D) Shortened platelet half-life in mice carrying mutations in Bcl-x is an intrinsic defect. Biotinylated platelets from mice of the indicated genotypes were adoptively transferred into unmanipulated recipients of the indicated genotypes, and their clearance from the circulation measured as in (B). (E) Loss of Bcl-x_{L} increases platelet turnover, resulting in a proportionately younger platelet population. The percentage of reticulated platelets was determined by staining with thiazole orange (Kienast et al., Blood, 75:116-121, 1990); each symbol represents an individual mouse. (F) Platelet production is not impaired by mutations in Bcl-x. Absolute numbers of reticulated platelets were determined by thiazole orange staining and measuring platelet count at steady state. Data in (B), (C) and (D) represent means±SD of 5-8 mice at each time point.

FIG. 3 provides a representation of data showing that the Bcl-x^{Plt20} and Bcl-x^{Plt16} mutations destabilize the Bcl-x_{L} protein (A) Location of the Plt20 and Plt16 mutations on Bcl-x_{L}. The two mutations (in blue) are mapped on the 3-dimensional structure of mouse Bcl-x_{L} (light gray) in complex with a Bim BH3 peptide (red) (1PQ1) (Liu et al. Immunity, 19:341-352, 2003). Y15 (Plt20) is partially solvent exposed, while I182 (Plt16) is completely buried, neither contributing to the BH3 binding groove of Bcl-x_{L}. The structural depiction was prepared using PyMOL (DeLano, W. L. 2002 The PyMOL Molecular Graphics System; http://www.pymol.org). (B) The mutant proteins encoded by the Plt20 and Plt16 alleles of Bcl-x can still bind Bax and Bak. FLAG-tagged wild type Bcl-x_{L}, or the Plt20 (Y15C) and Plt16 (I182N) mutants were co-expressed in 293T cells with HA-tagged Bax (upper) or Bak (lower), and the Triton X-100 containing lysates immunoprecipitated with the mouse monoclonal anti-FLAG (FL; M2 clone), -HA (HA.11 clone) or an irrelevant control antibody (-GluGlu). The blot was probed with rat monoclonal antibodies to FLAG (9H1) or HA (3F10). (C) The Plt20 and Plt16 mutations destabilize Bcl-x_{L}. Upper panels: decreased basal expression of Bcl-x_{L} Plt16 protein. Immunoblotting for Bcl-x_{L}, Mcl-1, Bak or Actin (loading control) using equivalent lysates prepared from primary MEFs of the indicated genotypes. Lower panels: equivalent lysates prepared from wild type or Bcl-x^{Plt20/Plt20} primary MEFs 0-24 h after exposure to 50 μg/mL cycloheximide (protein synthesis inhibitor) in the presence of the broad-spectrum caspase inhibitor qVD.OPh (50 μM) were probed for indicated proteins. Data shown is representative of at least 2 cell lines of each genotype analyzed. (D) Bcl-x^{Plt20} or Bcl-x^{Plt16} MEFs are susceptible to protein synthesis inhibition. The viability (determined by PI exclusion) of representative primary MEFs derived from wild type, Bcl-x^{Plt20/Plt20} or Bcl-x^{Plt16/Plt16} mice after exposure to 50 μg/mL cycloheximide for 0-30 h. Data represent means±SD of representative cell lines. †-<1% viability.

FIG. 4 provides a representation of data showing that the BH3 mimetic, ABT-737, triggers acute thrombocytopaenia. (A) Wild type C57BL/6 mice were injected with a single dose of ABT-737 (75 mg/kg; red arrow) given by intra-peritoneal injection. Animals were bled 2-24 h afterwards and platelet counts determined. All injected mice exhibited a significant reduction in platelet counts, with the nadir (<30% normal) occurring approximately 4 h after injection; each symbol represents a mouse. (B) Platelet recovery after a single dose of ABT-737. Platelet counts (blue symbols; left axis) were determined 2-96 h after a single dose of ABT-737 (red arrow). Note full recovery by day 3, and rebound thrombocytosis by day 4. Orange symbols represent serum TPO levels (right axis). (C) Cyclical acute thrombocytopaenia triggered by ABT-737. Platelet counts were determined before and 8 h after a single dose of ABT-737 (red arrows) given at weekly intervals. The drug caused a comparable drop in platelets each time. During recovery, baseline counts drifted upwards. (D) ABT-737 acts selectively on aged platelets. Representative flow cytometric profiles of thiazole orange stained platelets after a single dose of ABT-737 (red arrow); note increased proportion (% indicated in blue) of younger (reticulated) platelets after ABT-737 treatment. (E) Synchronizing platelets. A single dose of anti-platelet serum (APS; blue arrow) treatment provokes an acute, severe thrombocytopaenia (platelet counts: red symbols; left axis). During recovery, the newly synthesized younger platelets are larger as indicated by the increased mean platelet volume (blue symbols; right axis). (F) Young platelets are resistant to ABT-737. Wild type C57BL/6 mice were treated with APS, and then injected with ABT-737 (red arrows) either 2 or 7 days afterwards. Absolute platelet counts and the % of reticulated platelets were measured. The top panels show representative flow cytometric profiles following thiazole orange staining before or after ABT-737 injections. The bottom panels show platelet counts prior to or 2 h post ABT-737 injection. Data in (B), (C), (E) and (F) represent means±SD of 3-6 mice at each time point.

FIG. 5 provides a representation of data showing that the BH3 mimetic, ABT-737 triggers platelet apoptosis. (A) Expression of Bcl-2 family proteins in platelets. Lysates prepared from 50 μg or 5 μg plasma enriched for mouse platelets or MEFs, were probed for Bcl-x_{L}, Mcl-1, Bcl-2, Bak, Bax or Actin (loading control). (B) Genetic ablation of Bcl-x_{L} exacerbates ABT-737-induced thrombocytopaenia. Wild type C57BL/6, Bcl-x^{+/-} or Bcl-2^{+/-} mice were treated with a single dose of ABT-737 (75 mg/kg; red arrow) and the platelet counts determined 2-24 h afterwards. (C) ABT-737 triggers caspase activation in platelets. Immunoblotting for full-length intact caspase-3 (p32; top panel), cleaved p17 fragment (middle) or gelsolin (bottom) of cell lysates prepared from freshly isolated or cultured platelets that were left untreated or after exposure to ABT-737 (1 μM) with or without the broad-spectrum caspase inhibitor qVD.OPh (50 μM), qVD.OPh alone or Etoposide (10 μM). ABT-737 induced complete caspase-3 activation and gelsolin cleavage that was partially blocked by qVD.OPh. (D) ABT-737 triggers Bak-mediated caspase-dependent loss of platelets in culture. Wild type C57BL/6 or Bak^{-/-} platelets were counted 1 h after being left untreated, or after exposure to ABT-737 (1 μM), with or without qVD.OPh (50 μM), qVD.OPh alone or Etoposide (10 μM). Data represent means of normalized platelet counts (untreated=100%)±SD of 4 independent experiments, using platelets pooled from 6 mice of each genotype. (E) Human platelets exhibit caspase-dependent susceptibility to ABT-737 (1 μM for 1 or 2 h). (F) Absence of Bak protects platelets against ABT-737. Wild type C57BL/6, Bak^{-/-} and Bak^{-/-} Bax^{+/-} mice were treated with a single dose of ABT-737 (75 mg/kg; red arrow) and the platelet counts determined 0-24 h afterwards. Bak^{-/-} mice were unaffected by ABT-737 at early time points (up to 4 h), whereas Bak^{-/-}Bax^{+/-} mice were completely protected. Data in (B) and (F) represent means±SD of at least 6 mice at each time point.

FIG. 6 provides a representation of data showing that Bak is the major target of pro-survival Bcl-x_{L} in platelets. (A) Deletion of the gene encoding Bak results in thrombocytosis. Automated analysis of platelet counts in wild type C57BL/6, Bax^{-/-}, Bak^{-/-} or Bak^{-/-}Bax^{+/-} mice demonstrated that loss of Bak significantly elevated platelet numbers; Bax plays a less prominent role. (B) Normal platelet ultrastructure in Bak^{-/-} platelets. Transmission electron microscopic images of representative platelets from wild type (upper panel) or Bak^{-/-} (lower) mice. (C) Bak^{-/-} platelets have increased life-spans. Half-lives of platelets in mice of the indicated genotypes determined as described (data not shown). Data represent means±SD from 8 mice. (D) Genetic ablation of Bak prevents the thrombocytopaenia caused by loss-of-function mutations in Bcl-x_{L}. Platelet counts of mice with the indicated genotypes were compared. Deletion of one Bak allele prevented thrombocytopaenia in Bcl-x^{+/-} mice, whereas the loss of both alleles resulted in thrombocytosis indistinguishable from that caused by deletion of Bak alone. Thus, Bak lies genetically downstream of Bcl-x. (E) Thrombocytopaenia in heterozygous Bcl-x^{Plt20} or Bcl-x^{Plt16} mutant mice, on a mixed genetic background, was prevented by loss of Bak, and exacerbated by constitutive absence of Bcl-x. *p<0.05; statistical analyses in (A, D, E) were performed using two-tailed unpaired Student's t-test.

FIG. 7 provides a representation of data showing destablisation of Bcl-x_{L} in Bcl-x_{L}Plt20 and Bcl-x_{L}Plt16. (A) Polyclonal pools of Bcl-x^{-/-} MEFs stably expressing FLAG-tagged wild-type Bcl-x_{L} (blue histogram), Plt20 (green) or Plt16 (red) mutants were stained with an anti-FLAG antibody (M2) and immunofluorescence detected using a FITC-conjugated anti-mouse secondary antibody. Control staining (of vector infected MEFs) is shown by the black histogram. (B) Pools of cells described in (A) were treated with 50 μg/mL cycloheximide for 0-24 h and their viability determined by PI exclusion. Data show means from a representative experiment. (C) Viability of the pools described in (A) 24 h after treatment with 0-100 μM etoposide. Note the stronger protection afforded by wild type Bcl-x_{L} compared to that provided by Bcl-x_{L}^{Plt20} or Bcl-x_{L}^{Plt16}. Data show means±SD from a representative experiment. (D) Model for the regulation of Bak by Mcl-1 and Bcl-x_{L} (Willis et al., 2005 (supra)). Whereas Bim can bind to and neutralize bodi Mcl-1 and Bcl-x_{L} to cause Bak-mediated apoptosis, another BH3-only protein Noxa can only bind Mcl-1. Thus, Noxa cannot kill efficiently unless Bcl-x_{L} is also inactivated. (E) Bcl-x_{L}^{Plt16} has weak pro-survival activity. The viability of Bcl-x^{-/-} MEFs stably expressing wild type or mutant Bcl-x_{L} (as described in A) was determined by PI exclusion 24 h after infection with retrovirus expressing Bims or Noxa (Chen et al., Mol. Cell., 77:393-403, 2005). Bimₛ can counter the overexpression of wild type or mutant Bcl-x_{L} (gray columns). Noxa killed vector control Bcl-x^{-/-} MEFs by inactivating Mcl-1, the only remaining pro-survival protein controlling Bak. When wild type Bcl-x_{L} (or the Bcl-x_{L}^{Plt20} mutant) was introduced, Noxa could no longer kill because the overexpressed Bcl-x_{L}, which is spared by Noxa (see panel D), could keep Bak in check. By contrast, Bcl-x_{L}^{Plt16} was largely inert. Data show means±SD from a representative experiment.

FIG. 8 is a graphical representation of data showing the sensitivity of cell lines derived from Bcl-x mutant mice to apoptosis. (A) Primary MEFs derived from wild-type, Bcl-x^{Plt20/Plt20} or Bcl-x^{Plt16/Plt16} mice are equally sensitive to treatment with a broad-spectrum kinase inhibitor staurosporine (10 μM). Viability was determined by PI uptake; data represent means±SD of representative cell lines. \-<1% viability. (B) Factor-dependent myeloid (FDM) cells (Ekert et al., J. Cell. Biol., 755:835-842, 2004) derived from mice of the indicated genotypes were cultured in the presence of cycloheximide (100 ng/mL) for 1-48 h and their viability determined by PI exclusion and staining for Annexin V-FITC. Data represent means±SD of at least three independently derived cell lines tested in at least 3 independent experiments.

FIG. 9 is a graphical representation of data showing that ABT-737 does not affect platelet aggregation. Platelet aggregometry performed on human platelet rich plasma (250×10⁹/L platelets) pre-incubated for 1 h with 1 μM ABT-737 (green line) or not (red) before stimulation with (A) collagen (4 μg/ml) or (B) ADP (10 μM). Note that ABT-737 did not induce platelet aggregation or affect that with agonists. The BH3 mimetic compound did not alter responses to other agonists also tested, namely Epinephrine, Arachidonic Acid or Ristocetin (data not shown).

FIG. 10 is a structural representation of agents identified in the subject cellular screens for agents that enhance cellular viability, survival or life span.

### BRIEF DESCRIPTION OF THE TABLES

Table 1 provides a description of the SEQ ID NOs referred to in the specification.

Table 2 provides an amino acid sub-classification.

Table 3 provides exemplary amino acid substitutions.

Table 4 provides a list of non-natural amino acids contemplated in the present invention.

Table 5 shows the effect of ABT-737 on formation of hematopoietic progenitors in vitro. Cells (25,000) from the bone marrow of C57BL/6 mice were cultured in soft agar with G-CSF, IL-3 and EPO for 7 r d, stained and counted. Saline or ABT-737 was added on days 1, 3 and 5. There was a minor effect on megakaryocyte colony formation at the highest concentration used, while the formation of other colonies was unaffected. G, granulocyte colony; GM, granulocyte/macrophage colony; M, macrophage colony; E_{O}, eosinophil colony; Meg, megakaryocyte colony. Total colony counts are the means of two cultures; megakaryocyte colony counts represent the means±SD of four indepdendent cultures.

Table 6 provides the results of haemtological analysis of mice carrying mutant alleles of Bcl-x.

### DETAILED DESCRIPTION

The present invention is predicated in part upon the surprising and unexpected discovery that platelet survival can be modulated in vitro and in vivo using a physiological or pharmacological agent that modulates an intrinsic apoptotic program.

The present invention, therefore, provides methods of modulating the number and/or survival of platelets. The methods comprise administering an effective amount of an agent that modulates apoptosis. The agents promote apoptosis or reduce apoptosis in a cell, tissue or subject directly or via inhibition or potentiation of molecules that themselves directly promote or reduce apoptosis. By targeting conserved mediators of apoptosis, platelet survival may be prolonged or reduced.

Each embodiment described in this specification is to be applied mutatis mutandis to every other embodiment unless expressly stated.

As the skilled person will appreciate, various regulators or effectors of the apoptosis program may be targeted in accordance with the present invention, including apoptosis inducers, inhibitors and in some embodiments, effectors such as the caspase family of effector molecules.

The Bcl-2 family of molecules and its role in apoptosis has been the subject of considerable study. A range of apoptosis modulators have been identified (see for example, review articles by Baell, Biochem. Pharmacol, 64(5-6):851-863, 2002; Fesik, Nat. Rev. Cancer, 5(11):876-885, 2005; Schimmer, Cell Death Differ., 13(2):179-188, 2006).

For example, agents that promote apoptosis include: BH3 mimetic agents such as: peptides (see for example, Cosulich et al., Current Biology, 7:913-920, 1997; Diaz et al., J. Biol. Chem., 272:11350-11355, 1997; Holinger et al., J. Biol Chem., 274:13298-13304, 1999; Ottilie et al., Journal of Biological Chemistry, 272:30866-30872, 1997; Schimmer et al. Cell Death Differ., 8:725-733, 2001; Shangary, Biochemistry, 41:9485-9495, 2002; Wang et al., Cancer Research, 60:1498-1502, 2000b); constrained peptides (see for example, Walensky et al., Science, 305:1466-1470, 2004 & WO 2004/058804 incorporated herein in its entirety by reference); foldamers (see for example Sadowsky, J. Am. Chem. Soc., 727(34):11966-11968, 2005); and small organic compounds such as: Antimycin A (e.g. Tzung et al., Nat. Cell. Biol., 3:183-191, 2001); BH3I (e.g. Degterev et al., Nat. Cell. Biol., 3:173-182, 2001); Tetrooarcin A (e.g. Nakashima et al., Cancer Research, 60:1229-1235, 2000); Polyphenols including gossypol, (e.g. Kitada et al., J. Med. Chem., 46:4259-4264, 2003); Apogossypol (e.g. Becattini, 2004); HA14-1 (e.g. Wang et al., Proc. Natl. Acad. Sci. U.S.A., 97:7124-7129, 2000a); Compound 6 (e.g. Enyedy et al., J. Med. Chem., 44:4313-4324, 2001); ABT-737 (e.g. Oltersdorf et al., Nature, 435:677-681, 2005); terphenyl-based compounds (e.g. Yin, J. Am. Chem. Soc., 127(15):5463-5468, 2005); Benzoylurea compounds acting as alpha-helical mimics as disclosed in WO 2006/002474 incorporated herein in its entirety by reference; and Benzothiozole derivatives as disclosed, for example, in U.S. Ser. No. 60/789,982 filed 6 Apr. 2006 incorporated herein in its entirety by reference.

In another approach, Inhibitors of Apoptosis (IAP) molecules that inhibit apoptosis by inhibiting caspase activity are targeted. IAP antagonists, also known as SMAC/Diablo agonists are described, for example, by Oost, J. Med. Chem., 47(18):4417-4426, 2004; Wang, J. Biol. Chem., 279(46):48168-48176, 2004; Vucic, Biochem. J., 355(Pt 1):11-20, 2005; and Franklin, Biochemistry, 42(27):8223-8231, 2003.

Before describing the present invention in detail it is to be understood that unless otherwise indicated, the subject invention is not limited to specific formulations of components, screening methods, dosage regimens, or the like, as such may vary. It is also to be understood that the terminology used herein is for the purpose of describing particular embodiments only and is not intended to be limiting.

As used in this specification, the singular forms “a”, “an” and “the” include plural aspects unless the context clearly dictates otherwise. Thus, for example, reference to a “Bcl-2 family member” includes a single Bcl-2 family member, as well as two or more Bcl-2 family members; and so forth.

The terms “compound”, “molecule”, “active agent”, “pharmacological agent” or “physiological agent”, “medicament”, “agent” and “drug” are used to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, enantiomers, metabolites, analogs, etc. The term “agent” is not to be construed as an inorganic chemical compound only but extends to peptides, polypeptides and proteins as well as genetic molecules such as RNA, DNA and chemical analogs thereof. The term “modulator” is an example of an agent, molecule, pharmacologically active agent, medicament, active and drug which modulates apoptosis.

An “effective amount” means an amount necessary to at least partially attain the desired response. An effective amount for a human subject lies in the range of about 0.1 ng/kg body weight/dose to 1 g/kg body weight/dose. In some embodiments, the range is about 1μ, to 1 g, about 1 mg to 1 g, 1 mg to 500 mg, 1 mg to 250 mg, 1 mg to 50 mg, or 1μ to 1 mg/kg body weight/dose. Dosage regimes are adjusted to suit the exigencies of the situation and may be adjusted to produce the optimum therapeutic dose. For example, several doses may be provided daily, weekly, monthly or other appropriate time intervals,

Reference to “modulating”, “modulated” or “modulator” and the like includes down modulating, inhibiting antagonising, decreasing or reducing and up modulating, increasing, potentiating, agonising, prolonging, stimulating or enhancing as well as agents that have this effect. These terms are used herein with particular reference to “apoptosis”, survival, life-span, half-life, viability and cell function or activity. One or more of these attributes of a cell may be assessed or quantified using a range of cellular assays. For example, a number of different platelet function assays are described in the Examples such as Example 10.

In one embodiment, the present invention provides a method of modulating the number and/or survival of platelets or their precursors in a subject, the method comprising administering to the subject an effective amount of an agent that modulates the activity of Bcl-x_{L} and/or Bak and/or Bax polypeptide or functional variants thereof or that modulates the interaction between Bcl-x_{L} and Bak and/or Bax polypeptides. In another embodiment, the agent modulates the activity of Bax and/or Bak polypeptides.

Any subject or animal that could benefit from the present methods or compositions is encompassed. The term “subject” includes, without limitation, humans and non-human primates, animals, livestock animals, companion animals, laboratory test animals, captive wild animals, reptiles and amphibians, fish, birds etc. The most preferred subject of the present invention is a human subject. A subject, regardless of whether it is a human or non-human organism may be referred to as a patient, individual, subject, animal, host or recipient.

Reference to “interaction” between Bcl-x_{L} and Bak and/or Bax polypeptides and functional variants thereof includes, without limitation, binding between Bcl-x_{L} and Bak and/or Bax or functional variants or homologs thereof and interaction via one or more intermediary Bcl-2 family members. Binding between Bcl-x_{L} and Bak and/or Bax occurs for example via BH3 domains and thus agents that mimic the BH3 binding activity of Bak and/or Bax also modulate die interaction between Bcl-x_{L} and Bak and/or Bax. In some embodiments, agents that antagonise Bcl-x_{L} effectively agonise the activity of Bak and/or Bax and agents that antagonise Bak and/or Bax effectively agonise Bcl-x.

In another embodiment, the present invention provides a method of enhancing the number and/or survival of platelets or their precursors in a subject comprising administering an effective amount of an agent that enhances the level or activity of Bcl-x_{L} or that decreases the level or activity of Bak and/or Bax (in platelets), or that decreases the level or activity of caspases.

In yet another embodiment, the invention provides a method of decreasing the number and/or survival in platelets or their precursors, said method comprising administering an effective amount of an agent to a subject that reduces the level or activity of Bcl-x_{L} or that increases the level or activity of Bak and/or Bax in platelets or that increases the level or activity of caspases.

In another aspect, the present invention provides a method of enhancing platelet survival in vitro comprising contacting platelets in vitro with an agent that down modulates apoptosis. In one embodiment, the agent modulates the activity of Bcl-x_{L} or Bak and/or Bax. In another embodiment, the agent inhibits the activity of caspase enzymes or promotes the activity of endogenous inhibitors of apoptosis proteins such as IAP polypeptides.

Reference to modulating the “activity” of a target (including for example, a peptide, polypeptide, nucleic acid or cell) includes reference to the level or number of molecules/cells or the concentration of the target or the functional activity of the target or cell. Preferred targets belong to the Bcl-2 family of polypeptides or their encoding genetic sequences or down stream apoptosis effector molecules. Of these, Bcl-x_{L}, Bax and Bak and the Bcl-x_{L}/Bak/Bax pathway are particularly considered.

The activity of a polypeptide may be enhanced by increasing the level of transcription or translation of an encoding DNA or RNA. The activity of a polypeptide may also be decreased by reducing the level of transcription or translation such as by inhibiting promoter or enhancer activity or by the use of antisense/siRNA strategies now routine in the art. Accordingly, in some embodiments, the level of pro-survival or pro-apoptosis polypeptides in a platelet may be modulated by administering agents from which the polypeptide or its regulators are producible, such as a genetic construct encoding a functional form of the polypeptide. In another embodiment, the genetic/targeting construct encodes a regulator of expression of the target polypeptide such as an antisense molecule, promoter or enhancer. Those skilled in the art to which the present invention pertains will appreciate that a large number of strategies are available for delivering genetic constructs or polypeptide/peptide constructs to within a cell for modulating the activity of a polypeptides or of a portion of nucleic acid in a cell.

The terms “genetic material”, “genetic construct” “genetic forms”, “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

The present invention further contemplates recombinant nucleic acids including a recombinant construct comprising all or part of Bcl-x or Bak or Bax genes or functional variants of either of these. The recombinant construct may be capable of replicating autonomously in a host cell. Alternatively, the recombinant construct may become integrated into the chromosomal DNA of the host cell. Such a recombinant polynucleotide comprises a polynucleotide of genomic, cDNA, semi-synthetic or synthetic origin which, by virtue of its origin or manipulation: (i) is not associated with all or a portion of a polynucleotide with which it is associated in nature; (ii) is linked to a polynucleotide other than that to which it is linked in nature; or (iii) does not occur in nature. Where nucleic acids according to the invention include RNA, reference to the sequence shown shoidd be construed as reference to the RNA equivalent with U substituted for T. Such constructs are useful to elevate Bcl-x_{L} or Bak levels or to down-regulate Bcl-x_{L} or Bak and/or Bax levels such as via antisense means or RNAi-mediated gene silencing. As will be well known to those of skill in the art, such constructs are also useful in generating animal models and cells carrying modified alleles of Bcl-x_{L} or Bak and/or Bax. Such animals and cells and compositions comprising them are described briefly towards the end of the description.

As know to those of skill in the art, antisense polynucleotide sequences are useful agents in preventing or reducing the expression of endogenous or physiological regulators of apoptosis. Alternatively, morpholines may be used as described by Summerton et al. (Antisense and Nucleic acid Drug Development, 7:187-195, 1997). Antisense molecules may interfere with any function of a nucleic acid molecule. The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of pro-survival or pro-apoptosis regulators of apoptosis such as Bcl-x_{L} and/or Bak.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products.

In the context of the subject invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases. Typically, nuclease-resistant phosphorothioates that hybridise to nucleotides within the open reading frame of Bcl-x or Bak and/or Bax mRNA will induce RNAseH-mediated degradation.

The genetic agents or compositions in accordance with this aspect of the invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74,75, 76, 77, 78, 79, or 80 nucleobases in length.

The agents of the present invention in some embodiments comprise Bcl-x_{L} or Bak or Bax or functional fragments or functional variants thereof, or in genetic form as Bcl-x or Bak or Bax genes or functional parts or functional variants thereof or complementary forms of these.

The present invention provides a composition comprising Bcl-x or Bak or Bax, or Bcl-x_{L} or Bak or Bax (i.e., the molecule in genetic or proteinaceous form) or a functional variant thereof which substantially enhances or reduces the activity of Bcl-x_{L} or Bak and/or Bax for use in modulating interactions involving platelets and/or the survival of platelets. Compositions may be designed for in vitro or in vivo applications.

The modulatory agents of the present invention may be chemical agents such as a synthetic or recombinant molecules, polypeptides, peptides, modified peptides or proteins, lipids, glycoproteins or other naturally or non-naturally occurring molecules, variants, derivatives or analogs thereof. Alternatively, genetic agents such as DNA (gDNA, cDNA), RNA (sense RNAs, antisense RNAs, mRNAs, tRNAs, rRNAs, small interfering RNAs (SiRNAs), short hairpin RNAs (shRNAs), micro RNAs (miRNAs), small nucleolar RNAs (SnoRNAs), small nuclear (SnRNAs) ribozymes, aptamers, DNAzymes or other ribonuclease-type complexes may be employed.

Agents in accordance with this aspect of the invention may directly interact with Bcl-x_{L}, Bcl-x, Bak, Bak, Bax or Bax. Here, for example, antibodies or peptides, oligosaccharides, foldamers, peptidomimetics or analogs and other such biomolecules may be conveniently employed. Alternatively, genetic mechanisms are used to indirectly modulate the activity of Bcl-x_{L}, Bcl-x, Bak, Bak, Bax or Bax. Again, various strategies and reagents are well documented and include mechanisms for pre or post-transcriptional silencing. The expression of antisense molecules or co-suppression or RNAi or siRNA or shRNA or DNA strategies are particularly contemplated.

Aptamers are also contemplated. RNA and DNA aptamers can substitute for monoclonal antibodies in various applications (Jayasena, Clin. Chem., 45(9):1628-1650, 1999; Morris et al., Proc. Natl. Acad. Sci., USA, 95(6):2902-2907, 1998). Aptamers are nucleic acid molecules having specific binding affinity to non-nucleic acid or nucleic acid molecules through interactions other than classic Watson-Crick base pairing. Aptamers are described, for example, in U.S. Pat. Nos. 5,475,096; 5,270,163; 5,589,332; 5,589,332; and 5,741,679. An increasing number of DNA and RNA aptamers that recognize their non-nucleic acid targets have been developed by SELEX and have been characterized (Gold et al., Annu. Rev. Biochem., 64:763-797.1995; Bacher et al., Drug Discovery Today, 3(6):265-273, 1998).

In some embodiments, as discussed above, agents which modulate the level or activity of Bcl-x or Bak or Bax genes or Bcl-x or Bak or Bax polypeptides may be derived from Bcl-x, Bcl-x, Bak or Bak, Bax or Bax or be variants thereof. Alternatively, they may be identified in in vitro or in vivo screens. Natural products, combinatorial synthetic organic or inorganic compounds, peptide/polypeptide/protein, nucleic acid molecules and libraries or phage or other display technology comprising these are all available to screen or test for suitable agents. Natural products include those from coral, soil, plant, or the ocean or Antarctic environments.

Various domains of Bcl-2 family members may be specifically targeted or screened, such as the Bcl-2 homology domains, BH1, BH2, BH3 or BH4 domains of pro-apoptotic or pro-survival Bcl-2 family polypeptides. In some embodiments, the BH3 binding region of Bcl-2 proteins or the Bcl-2 binding region of Bak or Bax protein are targeted. In other embodiments the BH1 or BH1 domains may be targeted. Alternatively, unique regions of target proteins may be targeted to provide greater selectivity. Thus, in some embodiments, variants of Bak or Bax are contemplated that inactivate Bcl-x_{L} in platelets

In some embodiments, the agent to be tested is contacted with a system comprising Bcl-x_{L}, Bak or Bax polypeptides or peptides or Bcl-x, Bak or Bax genetic sequences. Then, the following may be assayed for: the presence of a complex between the agent and the target, a change in the activity of the target, or a change in the level of activity of an indicator of the activity of the target. Competitive binding assays and other high throughput screening methods are well known in the art and are described for example in International Publication Nos. WO 84/03564 and WO 97/02048; Bcl-x_{L} binding molecules are described, for example, in WO 2002/072761. In some embodiments, Bcl-x_{L} is used as a competitive binder of Bak and/or Bax. In other embodiments, Bak and/or Bax is/are used as a competitive binder to Bcl-x_{L}.

In another embodiment, cellular assays are used to identify compounds that maintain platelet viability. Such methods comprise incubating cells that are sensitive to apoptosis inducing agents in the presence of a compound to be tested, then contacting the cells with an apoptosis inducing agent and determining the presence of live cells that have not undergone apoptosis. In some embodiments, the cells are sensitive to antagonists of one or more members of the Bcl-2 family (including, for example, Bcl-2, Bcl-x_{L}, Bcl-w, Mcl-1 and A1) such as BH3 domain mimicking agents. In other embodiments, the cells are sensitive to Bcl-x_{L} or Mcl-1 antagonists, In another embodiment the Bcl-x_{L} antagonist is ABT-737. In some embodiments, the cells are platelets or fibroblasts such as mouse embryo fibroblasts (MEFs). In a preferred embodiment, the cells are cells in which the level or activity of Mcl-1 or Bcl-x_{L} is down regulated either in part or in full, generated by methods known in the art In some embodiments, Mcl-1 or Bcl-x_{L} levels are down regulated using chemical, genetic or gene silencing (RNAi) methods. For example, Mcl-1 levels can be reduced using CDK inhibitors (e.g. R-roscovitine) or protein synthesis inhibitors (e.g. cycloheximide). Genetic strategies include creation of loss of function alleles through deletion of all or part of a gene or through insertion of foreign DNA into a gene or through expression of a transgene from an exogeneous promoter. Conditional mutant technology may also be employed. Gene silencing offers a convenient procedure for inhibiting the function of genes. Mcl-1 antisense oligonucleotides are described, for example, in International Publication No. WO 2006/099667 incorporated herein in its entirety. Bcl-x_{L} level or activity is conventiently reduced using ABT-737 or an equivalent BH3 domain mimicking agent.

Thus, in some embodiments, the invention provides a method of identifying compounds that maintain platelet viability comprising incubating cells that are sensitive to Bcl-x_{L} or Mcl-1 antagonists in the presence of a compound to be tested, contacting said cells with a Bcl-x_{L} or Mcl-1 antagonist and determining the presence of live cells indicating that the compound is capable of blocking Bcl-x_{L} or Mcl-1 antagonist-inducing cell death and maintaining cell viability. In another embodiment, the Bcl-x_{L} antagonist is ABT-737 or an analog thereof. In some embodiments, cells that are sensitive to Bcl-x_{L} antagonists are Mcl-1 deficient. In other embodiments, cells that are sensitive to Mcl-1 antagonists are Bcl-x_{L} deficient. Compounds identified through initial screens are then tested to determine upon which targets they act. For example, compounds are tested in Mcl-1 null, Bax-null cells and Mcl-1 null, Bak-null cells to confirm that the compounds act via Bax or Bak, or a further downstream target. If required, further downstream targets are then tested in this manner.

In one embodiment a method of screening for a molecule which enhances the survival, lifespan or viability of platelets and/or other mammalian cells is considered, said method comprising: (i) combining the molecule with a cell; (ii) contacting the cell with one or more agents that antagonise pro-survival Bcl-2 family molecules in the cell and induce/s apoptosis; (iii) determining the change in survival (viability, lifespan, half-life) of cells in the presence of the molecule relative to a control; and (iv) selecting a molecule which enhances cell survival (viability, half-life). In some embodiments, the method comprises combining the selected molecule from (iv) with platelets to determine the change in cell survival (viability, half-life) of platelets in the presence of the molecule relative to controls. In other embodiments, the method comprises combining the selected molecule from (iv) with a target cell type to determine the change in cell survival (viability, half-life) of the cell in the presence of the molecule, relative to controls.

In another embodiment, a method for screening for a molecule which modulates apoptosis of a cell is contemplated, comprising: (a) combining the molecule and the cell; and (b) identifying modulation of a Bcl-2 family protein of the cell, wherein modulation of the Bcl-2 family protein indicates that the molecule modulates apoptosis of the cell.

In yet another embodiment, method of screening for a molecule which modulates a Bcl-2 family protein of a cell is contemplated, comprising: (a) combining the molecule and the cell; and (b) identifying whether apoptosis of the cell is modulated, wherein modulation of apoptosis indicates that the molecule modulates the Bcl-2 family protein.

In some embodiments the methods of the invention further comprise the step, between steps a) and b), of treating the cell to induce apoptosis. The cell may be treated with an agent which reduces the level and/or activity of a pro-survival member of the Bcl-2 protein family, such as Bcl-x_{L} and/or Mcl-1. In some embodiments the cell is treated with an agent which reduces the level and/or activity of a pro-apoptotic member of the Bcl-2 family, such as Bak and/or Bax.

In some further embodiments, the level and/or activity of at least one pro-survival member of the Bcl-2 family is reduced in the cell of step a). The level and/or activity of between one and six members of the Bcl-2 family selected from the group consisting of Bcl-x_{L}, Bcl-2, Bcl-w, Mcl-1, A1 and Bcl-B may be reduced in the cell of step a). The level and/or activity of Bcl-x_{L} and/or Mcl-1 may be reduced. In some embodiments, the level and/or activity of at least one pro-apoptosis member of the Bcl-2 protein family is reduced in the cell of step a), for example the level and/or activity of Bak and/or Bax may be reduced. In an illustrative embodiment, the level and/or activity of Mcl-1 and Bak are reduced. In other embodiments the level and/or activity of Mcl-1 and Bax are reduced.

In some embodiments, the methods occur in vitro. In other embodiments the method occurs in vivo.

High-throughput screening protocols are well used such as those described in Geysen (International Publication No. WO 84/03564). Briefly, large numbers of, for example, small peptide test compounds are synthesized on a solid substrate, such as plastic pins or some other surface. Bound polypeptide is detected by various methods. A similar method involving peptide synthesis on beads, which forms a peptide library in which each bead is an individual library member, is described in U.S. Pat. No. 4,631,211 and a related method is described in International Publication No. WO 92/00091. A significant improvement of the bead-based methods involves tagging each bead with a unique identifier tag, such as an oligonucleotide or electrophoretic tag, so as to facilitate identification of the amino acid sequence of each library member. These improved bead-based methods are described in International Publication No. WO 93/06121.

Another chemical synthesis screening method involves the synthesis of arrays of peptides (or peptidomimetics) on a surface wherein each unique peptide sequence is at a discrete, predefined location in the array. The identity of each library member is determined by its spatial location in the array. The locations in the array where binding interactions between a predetermined molecule and reactive library members occur are determined, thereby identifying the sequences of the reactive library members on the basis of spatial location. These methods are described in U.S. Pat. No. 5,143,854; International Publication Nos WO 90/15070 and WO 92/10092; Fodor et al., Science, 251:767, 1991. Of particular use are display systems, which enable a nucleic acid to be linked to the polypeptide it expresses. Selection protocols for isolating desired members of large libraries are known in the art, as typified by phage display techniques. Such systems, in which diverse peptide sequences are displayed on the surface of filamentous bacteriophage, are useful for creating libraries of antibody fragments (and the nucleotide sequences that encoding them) for the in vitro selection and amplification of specific antibody fragments that bind a target antigen. The nucleotide sequences encoding the V_{H} and V_{L} regions are linked to gene fragments which encode leader signals that direct them to the periplasmic space of E. coli and the resultant antibody fragments are displayed on the surface of the bacteriophage, typically as fusions to bacteriophage coat proteins (e.g., pIII or pVIII). Alternatively, antibody fragments are displayed externally on lambda phage capsids (phage bodies). An advantage of phage-based display systems is that selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward. Corresponding technologies are applied to combinatorial libraries of small organic molecules.

The three-dimensional structure of Bcl-x_{L}, Bak and Bax have been determined and this facilitates the design of binding agents that modulate apoptosis. Three-dimensional representations of the structure of one or more binding sites of Bcl-x_{L} and/or Bak and/or Bax or a variant, derivative or analog of either or these molecules to identify interacting molecules that, as a result of their shape, reactivity, charge potential etc. favourably interacts or associate. In a preferred aspect, the skilled person can screen three-dimensional structure databases of compounds to identify those compounds having functional groups that will fit into one or more of the binding sites. Combinational chemical libraries can be generated around such structures to identify those with high affinity binding to Bcl-x_{L}, Bak and/or Bax binding sites. Agents identified from screening compound databases or libraries are then fitted to three-dimensional representations of Bcl-x_{L}, Bak and/or Bax binding sites in fitting operations using, for example docking software programs,

A potential modulator may be evaluated “in silico” for its ability to bind to a Bcl-x_{L}, Bak or Bax active site prior to its actual synthesis and testing. The quality of the fit of such entities to binding sites may be assessed by, for example, shape complementarity by estimating the energy of the interaction. (Meng et al., J. Comp. Chem., 13:505-524, 1992).

The design of chemical entities that associate with components of the apoptosis pathway comprising Bcl-x_{L} and/or Bak and/or Bax generally involves consideration of two factors. Considering Bcl-x_{L} and Bak as examples, the compound must be capable of physically and structurally associating with Bcl-x_{L} or Bak. Non-covalent molecular interactions important in the association of Bcl-x_{L} or Bak with its interacting partners include hydrogen bonding, van der Waal's and hydrophobic interactions. Second, the compound must be able to assume a conformation that allows it to associate with Bcl-x_{L} or Bak. Although certain portions of the compound will not directly participate in this association with Bcl-x_{L} or Bak, those portions may still influence the overall conformation of the molecule. Such conformation requirements include the overall three-dimensional structure and orientation of the chemical entity or compound in relation to all or a portion of the active site, or the spacing between functional groups of a compound comprising several chemical entities that directly interact with Bcl-x_{L} or Bak.

Once a binding compound has been optimally selected or designed, as described above, substitutions may then be made in some of its atoms or side groups in order to improve or modify its binding properties. Generally, initial substitutions are conservative, i.e. the replacement group will have approximately the same size, shape, hydrophobicity and charge as the original group. It should of course be understood that components known in the art to alter conformation should be avoided.

Putative binding agents may be computationally evaluated and designed by means of a series of steps in which chemical entities or fragments are screened and selected for their ability to associate with the one or more binding sites. Selected fragments or chemical entities may then be positioned in a variety of orientations, or “docked,” to target binding sites. Docking may be accomplished using software, such as QUANTA and SYBYL, followed by energy minimization and molecular dynamics with standard molecular mechanics force fields, such as CHARMM or AMBER. Specialised computer programs may be of use for selecting interesting fragments or chemical entities. These programs include, e.g., GRID (Oxford University, Oxford, UK), 5 MCSS (Molecular Simulations, USA), AUTODOCK (Scripps Research Institute, USA), DOCK (University of California, USA), XSITE (University College of London, UK) and CATALYST (Accelrys).

Useful programs to aid the skilled addressee in connecting chemical entities or fragments include CAVEAT (University of California, USA), 3D database systems and HOOK (Molecular Simulations, USA) De-novo ligand design methods include those described in LUDI (Molecular Simulations, USA), LEGEND (Molecular Simulations, USA), LeapFrog (Tripos Inc.,) SPROUT (University of Leeds, UK) and the like.

Structure based ligand design is well known in the art and various strategies are available which can build on structural information to determine ligands which effectively modulate the activity of components of the apoptosis pathway comprising Bcl-x_{L}, Bak or Bax. Molecular modelling techniques include those described by Cohen et al., J. Med. Chem., 33:883-894, 1990, and Navia et al., Current Opinions in Structural Biology, 2:202-210, 1992.

Standard homology modelling techniques may be employed in order to determine the unknown three-dimensional structure or molecular complex. Homology modelling involves constructing a model of an unknown structure using structural coordinates of one or more related protein molecules, molecular complexes or parts thereof. Homology modelling may be conducted by fitting common or homologous portions of the protein whose three-dimensional structure is to be solved to the three-dimensional structure of homologous structural elements in the known molecule. Homology may be determined using amino acid sequence identity, homologous secondary structure elements and/or homologous tertiary folds. Homology modelling can include rebuilding part or all of a three-dimensional structure with replacement of amino acid residues (or other components) by those of the related structure to be solved.

Using such a three-dimensional structure, researchers identify putative binding sites and then identify or design agents to interact with these binding sites. These agents are then screened for a modulatory effect upon the target molecule.

In some embodiments, binding agents are designed with a deformation energy of binding of not greater than about 10 kcal/mole, more preferably not greater than 7 kcal/mole. Computer software is available to evaluate compound deformation energy and electrostatic interactions. For example, Gaussian 98, AMBER, QUANTA, CHARMM, INSIGHT II, DISCOVER, AMSOL and DelPhi.

Libraries of small organic molecules can be generated and screened using high-throughput technologies known to those of skill in this area. See for example U.S. Pat. No. 5,763,263 and US Application No. 20060167237. Combinatorial synthesis provides a very useful approach wherein a great many related compounds are synthesised having different substitutions of a common or subset of parent structures. Such compounds are usually non-oligomeric and may be similar in terms of their basic structure and function, varying in for example chain length, ring size or number or pattern of substitutions. Virtual libraries may also, as mentioned above, be constructed and compounds tested in silico (see for example, US Application No. 20060040322) or in vitro or in vivo assays known in the art.

In another aspect, agents are derived from nucleic acid molecules. In relation to nucleotide sequences of Bcl-x, Bak or Bax genes, the terms functional form or variant, functionally equivalent derivative or homolog include molecules which selectively hybridize to Bcl-x, Bak or Bax genes or a complementary form thereof over all or part of the genetic molecule under conditions of medium or high stringency at a defined temperature or range of conditions, or which have about 60% to 90% or 98% sequence identity to the nucleotide sequence defining Bcl-x, Bak or Bax genes.

Illustrative Bcl-x or Bak nucleotide sequences include those comprising nucleotide sequences set forth in SEQ ID NO: 1 or 5 or their complements or corrected forms (mouse or human Bcl-x_{L} mRNA) and SEQ ID NO: 3 or 7 (mouse or human Bak mRNA) or their complements or corrected forms. Illustrative Bax nucleotide sequences include those comprising nucleotide sequences set forth in SEQ ID NO: 9 or their complements or corrected forms. For the avoidance of doubt however, it should be noted that the term “Bcl-x gene” or “Bak gene” or “Bax gene” expressly encompass all forms of the gene including regulatory regions such as those required for expression of the coding sequence and genomic forms or specific fragments including probes and primers and constructs comprising same or parts thereof as well as cDNA or RNA and parts thereof.

The terms “functional form” or “variant”, “functionally equivalent derivatives” or “homologs” include polypeptides comprising a sequence of amino acids having about 60% sequence identity to the Bcl-x_{L} or Bak or Bax polypeptides of SEQ ID NO: 2, 4, 6, 8 or 10. Exemplary Bcl-x or Bak amino acid sequences include those comprising sequences set forth in SEQ ID NO: 2 (mouse Bcl-x_{L}) SEQ ID NO: 4 (mouse Bak), SEQ ID NO: 6 (human Bcl-x_{L}) or SEQ ID NO: 8 (human Bak). An exemplary Bax amino acid is set forth in SEQ ID NO: 10 (human Bax).

Reference herein to “medium stringency”, includes and encompasses from at least about 16% v/v to at least about 30% v/v fonnamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur et al., J. Mol. Biol., 5:109, 1962). However, the Tₘ of a duplex DNA decreases by 1° C. with every increase of 1% in the number of mismatch base pairs (Bonner et al, Eur. J, Biochem., 46:83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C. In some embodiments, the nucleic acid molecule encoding a Bcl-x_{L} or Bak polypeptide comprise a sequence of nucleotides as set forth in SEQ ID NOs: 1, 3, 5, 7 or 9 or which hybridises thereto or to a complementary form thereof under medium or high stringency hybridisation conditions. Preferably the hybridisation region is about 12 to about 80 nucleobases or greater in length.

More preferably, the percent identity between a particular nucleotide sequence and a reference sequence is about 60%, or 65% or about 70% or about 80% or about 85% or more preferably about 90% similarity or greater as about 95%, 96%, 97%, 98%, 99% or greater. Percent identities between 60% and 100% are encompassed.

A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al., Nucl. Acids Res. 25:3389, 1997. A detailed discussion of sequence analysis can be found in Unit 19.3 of Ausubel et al., Current Protocols in Molecular Biology John Wiley & Sons Inc, 1994-1998, Chapter 15).

A percentage of sequence identity between nucleotide sequences, for example, is calculated by comparing two optimally aligned sequences over the window of comparison, determining the number of positions at which the identical nucleic acid base (e.g. A, T, C, G, I) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity for amino acid sequences.

In some embodiments, the present invention contemplates the use of full-length Bcl-x_{L} or Bak or Bax or biologically active portions of those polypeptides. Biologically active Bcl-x_{L} or Bak or Bax portions comprise one or more binding domains. A biologically active portion of a full-length polypeptide can be a polypeptide which is, for example, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 120, 150, 300, or more amino acid residues in length, The Bcl-x_{L} or Bak or Bax polypeptides of the present invention includes all biologically active or functionally naturally occurring forms of Bcl-x_{L} or Bak or Bax as well as biologically active portions thereof and variants or derivatives of these. For example, Bcl-x_{L} or functional variants thereof including agonists or antagonists may be delivered to platelets in proteinaceous forms as part of a delivery construct designed to allow appropriate intracellular targeting.

The present invention also contemplates variant forms of the interacting molecules, “Variant” polypeptides include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is, they continue to possess at least one biological activity of the native protein. Such variants may result from, for example, genetic polymorphism or from human manipulation. Biologically active variants of a native Bcl-x, Bak or Bax polypeptide will have at least 40%, 50%, 60%, 70%, generally at least 75%, 80%, 85%, preferably about 90% to 95% or more, and more preferably about 98% or more sequence similarity with the amino acid sequence for the native protein as determined by sequence alignment programs described elsewhere herein using default parameters. A biologically active variant of a Bcl-x_{L} or Bak polypeptide may differ from that polypeptide generally by as much 100, 50 or 20 amino acid residues or suitably by as few as 1-15 amino acid residues, as few as 1-10, such as 6-10, as few as 5, as few as 4, 3, 2, or even 1 amino acid residue.

A Bcl-x_{L}, Bak or Bax polypeptide/peptide may be altered in various ways including amino acid substitutions, deletions, truncations, and insertions. Methods for such manipulations are generally known in the art. For example, amino acid sequence variants of a Bcl-x or Bak or Bax polypeptide can be prepared by introducing mutations in the encoding DNA. Methods for mutagenesis and nucleotide sequence alterations are well known in the art. See, for example, Kunkel (Proc. Natl. Acad. Sci. USA, 82:488-492, 1985), Kunkel et al. (Methods in Enzymol., 154:367-382, 1987), U.S. Pat. No. 4,873,192, Watson et al. (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (Natl. Biomed. Res. Found, 5:345-358, 1978). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of polypeptides. Recursive ensemble mutagenesis (REM), a technique that enhances the frequency of functional mutants in the libraries, can be used in combination with the screening assays to identify useful polypeptide variants (Arkin et al., Proc. Natl. Acad. Sci. USA, 89:7811-7815, 1992; Delgrave et al., Protein Engineering, 6:327-331, 1993). Conservative substitutions, such as exchanging one amino acid with another having similar properties, may be desirable as discussed in more detail below.

Variant Bcl-x_{L}, Bak or Bax polypeptides may contain conservative amino acid substitutions at various locations along their sequence, as compared to a reference amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan. As shown herein, substitution of tyrosine from the BH4 domain of Bcl-x_{L} or loss of isoleucine from the BH2 domain of Bcl-x_{L} polypeptide profoundly alters its ability to be active in vivo.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterises certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al, 1978 (supra); and by Gonnet et al., Science, 256(5062):1443-1445, 1992), however, include proline in the same group as glycine, serine, alanine and threonine. Accordingly, for the purposes of the present invention, proline is classified as a “small” amino acid.

Amino acid residues can be further sub-classified as cyclic or noncyclic, and aromatic or nonaromatic, self-explanatory classifications with respect to the side-chain substituent groups of the residues, and as small or large. The residue is considered small if it contains a total of four carbon atoms or less, inclusive of the carboxyl carbon, provided, an additional polar substituent is present; three or less if not. Small residues are, of course, always nonaromatic. Dependent on their structural properties, amino acid residues may fall in two or more classes. For the naturally-occurring protein amino acids, sub-classification according to this scheme is presented in the Table 2.

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional Bcl-x_{L} or Bak polypeptide can readily be determined by assaying its activity. Activities that can readily be assessed are known to those of skill and include assays to determine binding or dimerization or oliomerization detected by, for example, Biacore, kinetic, affinity and pull-down analyses. Conservative substitutions are shown in Table 3 below under the heading of exemplary substitutions. More preferred substitutions are shown under the heading of preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm. C. Brown Publishers (1993).

Thus, a predicted non-essential amino acid residue in a Bcl-x_{L} or Bak polypeptide is typically replaced with another amino acid residue from the same side chain family. Alternatively, mutations can be introduced randomly along all or part of the polynucleotide coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined.

Accordingly, the present invention also contemplates variants of the naturally-occurring Bcl-x_{L}, Bak or Bax polypeptide sequences or their biologically-active fragments, wherein the variants are distinguished from the naturally-occurring sequence by the addition, deletion, or substitution of one or more amino acid residues. In general, variants will display at least about 50, 55, 60, 65, 70, 75, 80, 85, 90, 91, 92, 93, 94, 95, 96, 97, 98, 99% identity to a reference Bcl-x_{L} or Bak polypeptide sequence as, for example, set forth in any one of SEQ ID NOs: 2, 4, 6, 8 or 10. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, 30, 40, 50 or more amino acids but which retain certain properties of the reference Bcl-x_{L} or Bak polypeptide are contemplated. The present variant Bcl-x_{L}, Bak, Bax polypeptides also include polypeptides that are encoded by polynucleotides that hybridize under stringency conditions as defined herein, especially high stringency conditions, to Bcl-x, Bak or Bax polynucleotide sequences, or the non-coding strand thereof.

In some embodiments, variant polypeptides differ from a Bcl-x_{L}, Bak or Bax polypeptide sequence by at least one but by less than 50, 40, 30, 20, 15, 10, 8, 6, 5, 4, 3 or 2 amino acid residue(s). In another, variant polypeptides differ from the corresponding sequence in any one of SEQ ID NOs: 2, 4, 6, 8 or 10 by at least 1% but less than 20%, 15%, 10% or 5% of the residues. If this comparison requires alignment the sequences should be aligned for maximum similarity. (“Looped” out sequences from deletions or insertions, or mismatches, are considered differences.) In one embodiments, the differences are differences or changes at a non-essential residue or a conservative substitution. A sequence alignment for Bcl-x_{L}, Bak or Bax proteins from a range of mammalian species is used to demonstrate conserved residues.

A “non-essential” amino acid residue is a residue that can be altered from the wild-type sequence of an embodiment polypeptide without abolishing or substantially altering one or more of its activities. Suitably, the alteration does not substantially alter one of these activities, for example, the activity is at least 20%, 40%, 60%, 70% or 80% of wild-type. An “essential” amino acid residue is a residue that, when altered from the wild-type sequence of an polypeptide agent of the invention, results in abolition of an activity of the parent molecule such that less than 20% of the wild-type activity is present.

In other embodiments, a variant polypeptide includes an amino acid sequence having at least about 50%, 55%, 60%, 65%, 70%, 75%, 80%, 85%, 90%, 91%, 92%, 93%, 94% 95%, 96%, 97%, 98% or more similarity to a corresponding sequence of a Bcl-x_{L} or Bak polypeptide as, for example, set forth in SEQ ID NOs: 2, 4, 6, 8 or 10, and has at least one activity of that Bcl-x_{L}, Bak or Bax polypeptide.

Polypeptide agents may be prepared by any suitable procedure known to those of skill in the art. For example, the polypeptides may be prepared by a procedure including the steps of: (a) preparing a chimeric construct comprising a nucleotide sequence that encodes at least a portion of a Bcl-x_{L}, Bak or Bax polypeptide or a functional variant thereof and that is operably linked to one or more regulatory elements; (b) introducing the chimeric construct into a host cell; (c) culturing the host cell to express the Bcl-x_{L}, Bak or Bax polypeptide or variant thereof; and (d) isolating the Bcl-x_{L}, Bak or Bax polypeptide or variant of either of these polypeptides from the host cell. In illustrative examples, the nucleotide sequence encodes at least a portion of the sequence set forth in SEQ ID NOs: 2, 4, 6, 8 or 10, or a variant thereof. Recombinant polypeptides can be conveniently prepared using standard protocols as described for example in Sambrook, et al., (1989, supra), in particular Sections 16 and 17; Ausubel et al, (1994, supra), in particular Chapters 10 and 16; and Coligan et al, CURRENT PROTOCOLS IN PROTEIN SCIENCE (John Wiley & Sons, Inc. 1995-1997), in particular Chapters 1, 5 and 6. Alternatively, polypeptides agents may be synthesised by chemical synthesis, e.g., using solution synthesis or solid phase synthesis as described, for example, in Chapter 9 of Atherton and Shephard (supra) and in Roberge et al., (Science, 269:202, 1995). The synthesis of conformationally constrained peptides is described for example in International Publication No, WO 2004106366.

The terms “derivative” or the plural “derivatives” and “variant” or “variants” are used interchangeable and, whether in relation to genetic or proteinaceous molecules, include as appropriate parts, mutants, fragments, and analogues as well as hybrid, chimeric or fusion molecules and glycosylafion variants. Particularly useful derivatives retain the functional activity of the parent molecule and comprise single or multiple amino acid substitutions, deletions and/or additions to the Bcl-x_{L}, Bak or Bax amino acid sequence. Preferably, the variants have functional activity or alternatively, modulate Bcl-x_{L}, Bak or Bax functional activity.

As used herein reference to a part, portion or fragment of Bcl-x, Bak or Bax genes is defined as having a minimal size of at least about 10 nucleotides or preferably about 13 nucleotides or more preferably at least about 20 nucleotides and may have a minimal size of at least about 35 nucleotides. This definition includes all sizes in the range of 10 to 35 as well as greater than 35 nucleotides. Thus, this definition includes nucleic acids of 12, 15, 20, 25, 40, 60, 100, 200, 500 nucleotides of nucleic acid molecules having any number of nucleotides between 500 and the number shown in SEQ ID NOs: 1, 3, 5, 7 or 9 or a complementary form thereof. The same considerations apply mutatis mutandis to any reference herein to a part, portion or fragment of Bcl-x_{L}, Bak or Bax polypeptide.

Substitutional variants typically contain the exchange of one amino acid for another at one or more sites within the protein and may be designed to modulate one or more properties of the polypeptide such as stability against proteolytic cleavage without the loss of other functions or properties. Amino acid substitutions may be made on the basis of similarity in polarity, charge, solubility, hydrophobicity, hydrophilicity and/or the amphipathic nature of the residues involved. Preferred substitutions are those which are conservative, that is, one amino acid is replaced with one of similar shape and charge. Conservative substitutions are well known in the art and typically include substitutions within the following groups: glycine, alanine; valine, isoleucine, leucine; aspartic acid, glutamic acid; asparagine, glutamine; serine, threonine; lysine, arginine; and tyrosine, phenylalanine.

Certain amino acids may be substituted for other amino acids in a protein structure without appreciable loss of interactive binding capacity with structures such as, for example, antigen-binding regions of antibodies or binding sites on substrate molecules or binding sites on proteins interacting with the polypeptide. Since it is the interactive capacity and nature of a protein which defines that protein's biological functional activity, certain amino acid substitutions can be made in a protein sequence and its underlying DNA coding sequence and nevertheless obtain a protein with like properties. In making such changes, the hydropathic index of amino acids may be considered. The importance of the hydrophobic amino acid index in conferring interactive biological function on a protein is generally understood in the art (Kyte et al., J. Mol. Biol., 157:105-132, 1982). Alternatively, the substitution of like amino acids can be made effectively on the basis of hydrophilicity. The importance of hydrophilicity in conferring interactive biological function of a protein is generally understood in the art (U.S. Pat. No. 4,554,101). The use of the hydrophobic index or hydrophilicity in designing polypeptides is further discussed in U.S. Pat. No. 5,691,198. The 3-D structures of various Bcl-2 proteins have been determined.

The term “homolog” or “homologs” refers herein broadly to functionally or structurally related molecules including those from other species. For example, viral homologs of Bcl-2 polypeptides have been described that antagonist Bcl-2 functions (White, Cell Death and Differentiation, 13:1371-1377, 2006).

Reference herein to “mimetics” includes carbohydrate, nucleic acid or peptide mimetics and it intended to refer to a substance which has conformational features allowing the substance to perform as a functional analog. A peptide mimetic may be a peptide containing molecule that mimic elements of protein secondary structure (Johnson et al., “Peptide Turn Mimetics” in Biotechnology and Pharmacy, Pezzuto et al., eds Chapman and Hall, New York, 1993). Peptide mimetics may be identified by screening random peptides libraries such as phage display or combinatorial libraries for peptide molecules which mimic the functional activity of Bcl-2 polypeptides. Alternatively, mimetic design, synthesis and testing are employed. The recognition of carbohydrates and lipids by proteins is an important event in many biological systems and the development of chemotherapeutics based on carbohydrate and/or lipid-mimics which can disrupt specific recognition processes is a rapidly emerging field. Nucleic acid mimetics include, for example, RNA analogs containing N3′-P5′ phosphoramidate internucleotide linkages which replace the naturally occurring RNA O3′-P5′ phosphodiester groups. Enzyme mimetics include catalytic antibodies or their encoding sequences, which may also be humanised.

Peptide or non-peptide mimetics can be developed as functional analogues of Bcl-x_{L}, Bak or Bax by identifying those residues of the target molecule which are important for function. Modelling can be used to design molecules which interact with the target molecule and which have improved pharmacological properties. Rational drug design permits the production of structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g., enhance or interfere with the function of a polypeptide in vivo. See, e.g. Hodgson (Bio/Technology, 9:19-21, 1991). In one approach, one first determines the three-dimensional structure of a protein of interest by NMR spectoscopy, x-ray crystallography, by computer modeling or most typically, by a combination of approaches. Useful information regarding the structure of a polypeptide may also be gained by modeling based on the structure of homologous proteins. In addition, putative peptide or polypeptide agents may be analyzed by an alanine scan (Weils, Methods Enzymol., 202:2699-2705, 1991). In this technique, an amino acid residue is replaced by Ala and its effect on the peptide's activity is determined. Each of the amino acid residues of the peptide is analyzed in this manner to determine the important regions of the peptide.

Mimics of BH3-only proteins are contemplated for their ability to bind to the hydrophobic grove of Bcl-x_{L} and there prevent Bcl-x_{L} from inhibiting Bak function. For example, as disclosed in WO 2006/002474 benzoylurea derivatives provide an alpha-helical peptidomimetic scaffold for interaction with Bcl-2 polypeptides. In another example, terphenyl derivatives provide an alpha-helical peptidomimetic of the Bak BH3 domain as described by Yin et al., 2005 (supra).

It is also possible to isolate a target-specific antibody selected by a functional assay and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore. As briefly described, it is possible to design or screen for mimetics which have enhanced activity or stability or are more readily and/or more economically obtained.

In some embodiments, analogs have enhanced stability and activity or reduced unfavourable pharmacological properties. They may also be designed in order to have an enhanced ability to cross biological membranes or to interact with only specific substrates. Thus, analogs may retain some functional attributes of the parent molecule but may posses a modified specificity or be able to perform new functions useful in the present context i.e., for administration to a subject.

In another aspect, analogs of agonist or antagonist agents are contemplated. Analogs of peptide or polypeptide agents contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with eyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetramtromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid contemplated herein is shown in Table 4.

Crosslinkers can be used, for example, to stabilize 3D conformations, using homo-bifunctional crosslinkers such as the bifunctional imido esters having (CH₂)ₙ spacer groups with n=1 to n=6, giutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids and the introduction of double bonds between C_{α} and C_{β} atoms of amino acids.

Conformationally constrained peptides are contemplated that modulate apoptosis, such as BH3-only protein mimics described in WO 2006/00034. Here, the helical conformation of molecules that bind to the hydrophobic pocket of Bcl-2 proteins are stabilized by means of a linker covalently bound between two amino acid residues in the sequence.

Agents for we in the present invention, such as peptides or small organic or inorganic molecules, carbohydrates, lipids or nucleic acid molecules can readily be conjugated to targeting compounds to allow direct delivery of agents to platelets or platelet precursors. Suitable targeting agents are known to those of skill in the art and include antibodies or antigen-binding fragments thereof. Antibodies and their generation and treatment are well known to those in the art. Exemplary protocols for their production are provided in Coligan et al “Current Protocols in Immunology” (John Wiley & Sons, 1991) and Ausubel et al “Current Protocols in Molecular Biology” (1994-1998). Antibodies may be polyclonal or monoclonal antibodies, fragments include Fv, Fab, Fab′ and F(ab′)₂ portions of immunoglobulin molecules. Synthetic Fv fragments are conveniently employed including synthetic single chain Fv fragments prepared, for example, as described in U.S. Pat. No. 5,091,513. Other binding molecules include single variable region domains (referred to as dAbs), or minibodies comprising a single chain comprising the essential elements of a complete antibody as disclosed in U.S. Pat. No. 5,837,821. In further embodiments, the antigen binding molecule comprises multiple binding sites for one or more antigens (e.g. multi-scFvs). In other embodiments, the antigen binding molecule is a non-immunoglobulin derived protein framework having complementary determining regions selected for a particular antigen such as a platelet surface protein moiety.

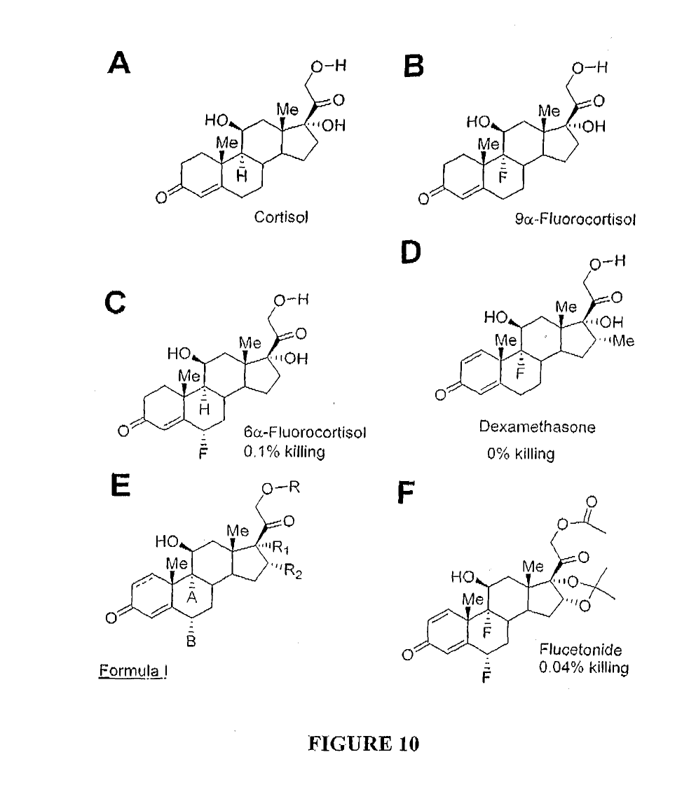

In another embodiment, a method is considered for enhancing or maintaining the viability or lifespan of platelets according to any one of claims 1 to 13, said method comprising administering an agent or composition comprising an agent of Formula 1 or an or a composition comprising an agent selected from one of agents (a) to (d) in FIG. 10, Thus, an apoptosis inhibitory or apoptosis retarding agent or a composition comprising same is contemplated for use in the treatment or prevention of thrombocytopaenia, said agent comprising the structure set out in Formula 1. In another embodiment, an apoptosis inhibitory or apoptosis retarding agent or composition comprising same is considered for use in enhancing the viability or survival of stored platelets, said agent comprising the structure set out in Formula 1. In another embodiment, the subject agents are used to preserve tissue or organ viability for transplantation.

The molecule identified by a method of the invention may modulate apoptosis. As used herein the term “modulate” means changed or adjusted. Thus the rate of apoptosis of the cell may be changed or adjusted. The rate of apoptosis may be increased or decreased. That is, the life of the cell may be made greater or lesser. Alternatively or in addition, the level and/or activity of a member of the Bcl-2 family of proteins may be modulated and may be increased or decreased. That is, the level and/or activity of the Bcl-2 family member may be made greater or lesser.

The molecule may modulate apoptosis and/or the level and/or activity of a member of the Bcl-2 family directly or indirectly. For example, the molecule may bind to a member of the Bcl-2 protein family. Alternatively the molecule may bind to another molecule which in turn binds to a member of the Bcl-2 protein family. For example, the molecule may indirectly modulate apoptosis and/or the level and/or activity of a member of the Bcl-2 protein family. The small molecule ABT-737 is a BH3 mimetic drug that antagonizes pro-survival Bcl-x_{L}. It selectively targets Bcl-2, Bcl-x_{L} and Bcl-w but not the other pro-survival proteins Mcl-1 or A1. Alternatively, the molecule may modulate apoptosis by binding to another molecule downstream from the Bcl-2 protein family, such as a caspase.

The molecule may be an agonist or antagonist of a member of the Bcl-2 protein family. As used herein the term “agonist” refers to a molecule that improves the activity of a different molecule. The term “antagonist” refers to a molecule that counteracts the action of another. Thus the molecule may upregulate or downregulate apoptosis and/or the level and/or activity of a member of the Bcl-2 family of proteins.

The molecule identified by a method of the invention will have use generally in preserving or maintaining cell viability, and especially mammalian cell viability, for example, in the treatment or prevention of range of conditions. In an illustrative example the following conditions are specifically contemplated: an apoptosis mediated disease, cytopaenia, an inflammatory disease, an autoimmune disease, a destructive bone disorder, a proliferative disorder, an infectious disease, a degenerative disease, a disease associated with cell death, an excess dietary alcohol intake disease, a viral mediated disease, uveitis, inflammatory peritonitis, osteoarthritis, pancreatitis, asthma, adult respiratory distress syndrome, glomerulonephritis, rheumatoid arthritis, systemic lupus erythematosus, scleroderma, chronic thyroiditis, Grave's disease, autoimmune gastritis, diabetes, autoimmune hemolytic anemia, autoimmune neutropenia, tlrrombocytopaenia, chronic active hepatitis, myasthenia gravis, inflammatory bowel disease, Crohn's disease, psoriasis, atopic dermatitis, scarring, graft vs host disease, organ transplant rejection, osteoporosis, leukemias and related disorders, myelodysplastic syndrome, multiple myeloma-related bone disorder, acute myelogenous leukemia, chronic myelogenous leukemia, metastatic melanoma, Kaposi's sarcoma, multiple myeloma, haemorrhagic shock, sepsis, septic shock, burns, Shigellosis, Alzheimer's disease, Parkinson's disease, Huntington's disease, prion disease, cerebral ischemia, epilepsy, myocardial, ischemia, acute and chronic heart disease, myocardial infarction, congestive heart failure, atherosclerosis, coronary artery bypass graft, spinal muscular atrophy, amyotrophic lateral sclerosis, multiple sclerosis, HIV-related encephalitis, aging, alopecia, neurological damage due to stroke, ulcerative colitis, traumatic brain injury, spinal cord injury, hepatitis-A, hepatitis-B, hepatitis-C, hepatitis-D, hepatitis-E, hepatitis-G, other forms of viral hepatitis, drug (e.g. paracetamol-induced liver disease, yellow fever, dengue fever, Japanese encephalitis, liver disease, alcoholic hepatitis, renal disease, polycystic kidney disease, H. pylori-associated gastric and duodenal ulcer disease, HIV infection, tuberculosis, meningitis, and to treat complications associated with coronary artery bypass grafts.

More specifically, a molecule identified by a method of the invention may be used to preserve organ viability such as without limitation a kidney, heart or heart valve, lung, liver, skin, cornea, vein and other vessels, bone, tendon and other musculo skeletal tissue, pancrease, intestine and the like. In some embodiments, a molecule so identified is used to prolong platelet survival in patients or in blood bank storage, as well as to treat or prevent myocardial infarcts, reperfusion injuries, thrombotic strokes to minimize loss of neuronal tissues, prevent gut toxicity (mucositis) following high-dose chemotherapy and total body radiation, hepatitis and other forms of liver failures, inflammatory diseases that lead to tissue loss e.g. rheumatoid arthritis, anemias, neutropenias, infertility due to loss of sperm viability, and premature greying due to loss of melanocytes (cells for hair pigmentation). In some embodiments, the cell is one other than a platelet cell.

The cell used to identify modulation of the level and/or activity of a member of the Bcl-2 family of proteins and/or apoptosis and the cell to be treated or whose life span is to be maintained or enhanced may be any cell winch comprises one or more members of the Bcl-2 protein family, that is, any cell of a multicellular organism. The cell may be from any multicellular organism as members of the Bcl-2 family of proteins, or homologies thereof, are found in organisms such as C. elegans, mice, and humans. Thus the cell may be from a human or a mammal of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels), horses, and birds including those kinds of birds that are endangered, kept in zoos, and fowl, and more particularly domesticated fowl, e.g., poultry, such as turkeys, chickens, ducks, geese, guinea fowl, and the like, as they are also of economical importance to humans. The term does not denote a particular age. Thus, cells from both adult and newborn organisms are intended to be covered.

The cell to be treated may be any cell having a nucleus including, without limitation, a fibroblast, neural cell, epithelial cell, endothelial cell, stem cell, hepatocyte, myoblast, osteoblast, osteoclast, lymphocyte, keratinocyte, mesothelial cell, and muscle cell. Alternatively the cell may be anuclear, that is, without a nucleus, and thus have no DNA. Examples of anuclear cells include red blood cells (erythrocytes) and platelets (thrombocytes).

In some embodiments the cell is deficient in one or more pro-survival members of the Bcl-2 protein family. In other embodiments the cell is deficient in one or more pro-apoptotic members of the Bcl-2 protein family. In some embodiments the cells are Mcl-1 deficient cells.

A cell deficient in a protein may be generated by methods known in the art. For example, the technique known as “gene disruption” selectively inactivates a gene in an otherwise normal cell by replacing the gene with a mutant allele. Powerful methods have been developed for accomplishing gene disruption (also called gene knockout or gene silencing) in the cells of organisms such as yeast and mice. These methods rely on the process of homologous recombination, in which regions of sequence similarity exchange segments of DNA. “Homologous recombination” refers to the exchange of nucleic acid regions between two nucleic acid molecules at the site of homologous nucleotide sequences. Foreign DNA inserted into a cell can disrupt any gene with which it is, at least in part, homologous by exchanging segments. Specific genes can be targeted if their nucleotide sequences are known.

In some embodiments the foreign DNA may be located on a targeting construct. A targeting construct is an artificially constructed segment of genetic material which can be transferred into selected cells. The targeting construct can integrate with the genome of the host cell in such a position so as to enhance or inhibit (partially or enthely) expression of a specific gene.

The targeting construct may be produced using standard methods known in the art. For example, as described in Sambrook and Russell, Molecular Cloning: A Laboratory Manual, 3^{rd} Edition, Cold Spring Harbor Laboratory Press, Cold Spring Harbor, N.Y., 2001; Ausubel (ed), Current Protocols in Molecular Biology, 5^{th} Edition, John Wiley & Sons, Inc, NY, 2002).

The development of the targeting construct facilitates its introduction into a cell to be used in a method of the invention. Various techniques for introducing a targeting construct into a host cell, either in vivo or in vitro, are known in the art and include, but are not limited to, microinjection, viral-mediated transfer, and electroporation.

In order to modulate apoptosis and/or the level and/or activity of a member of the Bcl-2 family of proteins, the molecule will be combined with a cell in vitro or in vivo. Combining the molecule and the cell may be achieved by any method known in the art. In some embodiments the cell has been isolated from the organism and combining the molecule and the cell occurs in vitro. In other embodiments the cell has not been isolated from the organism and combining the molecule and the cell occurs in vivo. The molecule may be combined with the cell directly, i.e., applied directly to the cell. Alternatively the molecule may be combined with the cell indirectly, e.g. by injecting the molecule into the bloodstream of an organism, which then carries the molecule to the cell.

A cellular assay may be used to identify molecules which modulate apoptosis and/or the level and/or activity of a member of the Bcl-2 family of proteins. Such methods comprise incubating cells which are sensitive to apoptosis-inducing molecules in the presence of a test molecule and determining the presence of live cells which have not undergone apoptosis. If the molecule modulates the level and/or activity of a member of the Bcl-2 protein family this may be identified by determining whether or not the cell has undergone apoptosis. Alternatively, if the molecule modulates apoptosis of the cell, this may be identified by determining the level and/or activity or a member of the Bcl-2 family of proteins.

Many different methods have been devised to detect apoptosis such as uptake of vital cellular dyes (eosin red, trypan blue, alamar blue), TUNEL (TdT-mediated dUTP Nick-End Labeling) analysis, ISEL (in situ end labeling), and DNA laddering analysis for the detection of fragmentation of DNA in populations of cells or in individual cells, Annexin-V staining that measures alterations in plasma membranes, detection of apoptosis related proteins such as caspases (including caspase activity or activation), Bcl-2 family proteins, p53, Fas and FADD. These are techniques known to the skilled person.

Similarly, many methods are known to the skilled person for detecting the level and/or activity of a member of the Bcl-2 protein family. For example, the protein can be purified from the cell, such as by chromatographic techniques, and compared to the protein purified from a cell which has not been subjected to the method of the invention.

The small or large chemicals, polypeptides, nucleic acids, antibodies, peptides, chemical analogs, or mimetics of the present invention can be formulated in pharmaceutic compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabiliser or other materials well known in the art. Such materials should be non-toxic and should not interfere with the efficacy of the active ingredient. The carrier may take a wide variety of forms depending on the form of preparation desired for administration, e.g. intravenous, oral, intrathecal, epineuval or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698. For parenteral administration, the compound may dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, (supra).

Alternatively, targeting therapies may be used to deliver the active agent more specifically to tissues producing or accumulating platelets such as the bone marrow, lung, spleen, vascular system by the use of targeting systems such as antibodies or cell specific ligands, vectors or model site of delivery. Targeting may be desirable for a variety of reasons, e.g. to avoid targeting other areas of the body, if the agent is unacceptably toxic or if it would otherwise require too high a dosage or if it would not otherwise be able to enter the target cells.

Instead of administering these agents directly, they could be produced in the target cell, e.g. in a viral vector such as those described above or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells or expression of expression products could be limited to specific cells, stages of development or cell cycle stages. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

In accordance with this aspect of the present invention, the cells of a subject exhibiting modified Bcl-x, Bak or Bax genetic sequences may be treated with a genetic composition comprising Bcl-x, Bak or Bax polynucleotide. The provision of wild type or enhanced Bcl-x_{L}, Bak or Bax function to a cell which carries a mutant or altered form of the gene should in this situation complement the deficiency. The wild type allele may be introduced into a cell in a vector such that the gene remains extrachromosomally. Alternatively, artificial chromosomes may be used. Typically, the vector may combine with the host genome and be expressed therefrom.

Gene therapy would be carried out according to generally accepted methods, for example, as described by Friedman (In: Therapy for Genetic Disease, T, Friedman, Ed., Oxford University Press, pp. 105-121, 1991) or Culver (Gene Therapy: A Primer for Physicians, 2^{nd} Ed., Mary Ann Liebert, 1996). Suitable vectors are known, such as disclosed in U.S. Pat. No. 5,252,479, International Patent Publication No. WO 93/07282 and U.S. Pat. No. 5,691,198. Gene transfer systems known in the art may be useful in the practice of the gene therapy methods of the present invention. These include viral and non-viral transfer methods. Non-viral gene transfer methods are known in the art such as chemical techniques including calcium phosphate co-precipitation, mechanical techniques, for example, microinjection, membrane fusion-mediated transfer via liposomes, direct DNA uptake, receptor-mediated DNA transfer and nucleofection. Viral-mediated gene transfer can be combined with direct in vivo gene transfer using liposome delivery.

Expression vectors in the context of gene therapy are meant to include those constructs containing sequences sufficient to express a polynucleotide that has been cloned therein. In viral expression vectors, the construct contains viral sequences sufficient to support packaging of the construct. If the polynucleotide encodes Bcl-x_{L}, for example, expression will produce Bcl-x_{L}. If the polynucleotide encodes a sense or antisense polynucleotide or a ribozyme or DNAzyme, expression will produce the sense or antisense polynucleotide or ribozyme or DNAzyme. Thus, in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters are routinely determined.

Receptor-mediated gene transfer may be achieved by conjugation of DNA to a protein ligand via polylysine. Ligands are chosen on the basis of the presence of the corresponding ligand receptors on the cell surface of the target cell/tissue type. Receptors on the surface of liver cells may be advantageously targeted. These ligand-DNA conjugates can be injected directly into the blood if desired and are directed to the target tissue where receptor binding and internalization of the DNA-protein complex occurs. To overcome the problem of intracellular destruction of DNA, co-infection with adenovirus can be included to disrupt endosome function.

In a further related aspect of the present invention it has been determined that alternations in the level or activity of Bcl-x_{L}, Bak or Bax have profound effects on platelet survival. Accordingly, susceptibility to conditions associated with subnormal or supernormal platelet numbers can now be diagnosed by monitoring subjects for modification in the level or activity of Bcl-x_{L} and/or Bak and/or Bax or specific mutations or aberrations (such a methylation events) in Bcl-x and/or Bak and/or Bax genes.

The term “gene” is used in its broadest sense and includes cDNA corresponding to the exons of a gene. Reference herein to a “gene” is also taken to include:
(i) a classical genomic gene consisting of transcriptional and/or translational regulatory sequences and/or a coding region and/or non-translated sequences (i.e. introns, 5′- and 3′-untranslated sequences); or
(ii) mRNA or cDNA corresponding to the coding regions (i.e. exons) and 5′- and 3′-untranslated sequences of the gene.

Mutations or other modifications to the gene may cause total or partial gain or loss of Bcl-x_{L}, Bak or Bax function. In some embodiments, modification in the gene affects transcription, translation or post-translational processing and so affects the level or activity of Bcl-x_{L}, Bak or Bax. In some embodiments, the mutation in Bcl-x is in the BH4 and/or BH1, BH2 or BH3 encoding domains; in Bak or Bax in the BH1, BH2 or BH3 domains.

A wide range of mutation detection screening methods are available as would be known to those skilled in the art. Any method which allows an accurate comparison between a test and control nucleic acid sequence may be employed. Scanning methods include sequencing, denaturing gradient gel electrophoresis (DGGE), single-stranded conformational polymorphism (SSCP and rSSCP, REF-SSCP), chemical cleavage methods such as CCM, ECM, DHPLC and MALDI-TOF MS and DNA chip technology. Specific methods to screen for pre-determined mutations include allele specific oligonucleotides (ASO), allele specific amplification, competitive oligonucleotide priming, oligonucleotide ligation assay, base-specific primer extension, dot blot assays and RFLP-PCR. The strengths and weaknesses of these and further approaches are reviewed in Sambrook, Chapter 13, Molecular Cloning, 2001. Methylation detection assays are also known in the art with methods for the detection of 5-methylcytosines being the most advanced, as reviewed by Rein et al., Nucleic Acids Research, 26(10):2255-2264, 1998. Detection of cytosine methylation is also described in International Publication Nos. WO 00/70090 and WO 03/000926.

The present invention provides methods of diagnosis of conditions associated with thrombocytopaenia or thrombocytoses in a subject and further provides genetic or protein based methods of determining the susceptibility of a subject to develop these conditions.

The diagnostic and prognostic methods of the present invention detect or assess an aberration in the wild-type Bcl-x and/or Bak and/or Bax gene or locus to determine if a modified polypeptide will be produced or if it will be over-produced or under-produced. The term “aberration” in the gene or locus encompasses all forms of mutations including deletions, insertions, point mutations and substitutions in the coding and non-coding regions. It also includes changes in methylation patterns of the gene. Point mutations may result in stop codons, frameshift mutations or amino acid substitutions. Somatic mutations are those which occur only in certain tissues, e.g. in the tumor tissue and are not inherited in the germline. Germline mutations can be found in any of a body's tissues and are inherited.

Predisposition to conditions associated with thrombocytopaenia or thrombocytoses can be ascertained by testing any tissue of a human or other mammal for mutations in a Bcl-x and/or Bak and/or Bax gene. The mutation can be determined by testing DNA from any tissue of a subject's body. In addition, pre-natal diagnosis can be accomplished by testing fetal cells, placental cells or amniotic fluid for mutations of the Bcl-x and/or Bak and/or Bax gene. Alteration of a wild-type allele whether, for example, by point, mutation or by deletion or by methylation, can be detected by any number of means.

Useful diagnostic techniques to detect aberrations in the Bcl-x and/or Bak and/or Bax gene include but are not limited to fluorescent in situ hybridization (FISH), PFGE analysis, Southern blot analysis, dot blot analysis and PCR-SSCP. Also useful is DNA microchip technology. Direct DNA sequencing, either manual sequencing or automated fluorescent sequencing, can detect sequence variation. Another approach is the single-stranded conformation polymorphism assay (SSCP) (Orita et al., Proc. Nat. Acad. Sci. USA, 86:2776-2770, 1989). This method can be optimized to detect most DNA sequence variation. The increased throughput possible with SSCP makes it an attractive, viable alternative to direct sequencing for mutation detection on a research basis. The fragments which have shifted mobility on SSCP gels are then sequenced to determine the exact nature of the DNA sequence variation. Other approaches based on the detection of mismatches between the two complementary DNA strands include clamped denaturing gel electrophoresis (CDGE) (Sheffield et al., Am. J. Hum. Genet., 49:699-706, 1991), heteroduplex analysis (HA) (White et al., Genomics, 12:301-306, 1992) and chemical mismatch cleavage (CMC) (Grompe et al., Proc. Natl. Acad. Sci. USA, 86:5855-5892, 1989). Other methods which might detect mutations in regulatory regions or which might comprise large deletions, duplications or insertions include the protein truncation assay or the asymmetric assay. A review of methods of detecting DNA sequence variation can be found in Grompe (Proc. Natl. Acad. Sci. USA, 86:5855-5892, 1993).

Other tests for confirming the presence or absence of a wild-type or mutant Bcl-x and/or Bak and/or Bax alleles; denaturing gradient gel electrophoresis (DGGE) (Wartell et al., Nucl. Acids Res., 18:2699-2705, 1990; Sheffield et al., Proc. Natl. Acad. Sci. USA, 86:232-236, 1989); RNase protection assays (Finkelstein et al., Genomics, 7:167-172, 1990; Kinszler et al., Science, 251:1366-1370, 1991); denaturing HPLC; allele-specific oligonucleotide (ASO hybridization) (Conner et al, Proc. Natl. Acad. Sci. USA, 80:278-282, 1983); the use of proteins which recognize nucleotide mismatches such as the E. coli mutS protein (Modrich, Ann. Rev. Genet., 25:229-253, 1991) and allele-specific PCR (Ruano et al., Nucl. Acids. Res. 17:8392, 1989). For allele-specific PCR, primers are used which hybridize at their 3′ ends to a particular. If the particular mutation is not present, an amplification product is not observed. Amplification Refractory Mutation System (ARMS) can also be used, as disclosed in European Patent Publication No. 0 332 435 and in Newtown et al. (Nucl. Acids. Res. 77:2503-2516, 1989).

Nucleic acid sequences of the Bcl-x and/or Bak and/or Bax gene(s), which have been amplified by use of PCR or other amplification reactions may also be screened using allele-specific probes. These probes are nucleic acid oligomers, each of which contains a region of the gene sequence harboring a known mutation. By use of a battery of allele-specific probes, PCR amplification products can be screened to identify the presence of a previously identified mutation in the Bcl-x and/or Bak and/or Bax gene. Hybridization of allele-specific probes with amplified Bcl-x and/or Bak and/or Bax sequences can be performed, for example, on a nylon filter. Hybridization to a particular probe under stringent hybridization conditions indicates the presence of the same mutation in the tissue as in the allele-specific probe.

Microchip technology is also applicable to the present invention. In this technique, thousands of distinct oligonucleotide or cDNA probes are built up in an array on a silicon chip or other solid support such as polymer films and glass slides. Nucleic acid to be analyzed is labelled with a reporter molecule (e.g. fluorescent label) and hybridized to the probes on the chip. It is also possible to study nucleic acid-protein interactions using these nucleic acid microchips. Using this technique, one can determine the presence of mutations or sequence the nucleic acid being analyzed or one can measure expression levels of a gene of interest or multiple genes of interest such as genes encoding products in a biochemical pathway. The technique is described in a range of publications including Hacia et al (Nature Genetics, 14:441-447, 1996) and Shoemaker et al. (Nature Genetics, 14:450-456, 1996).

Alteration of wild-type Bcl-x and/or Bak and/or Bax genes can also be detected by screening for alteration of wild-type Bcl-x, Bak or Bax proteins. For example, monoclonal antibodies immunoreactive with Bcl-x, Bak or Bax can be used to screen sample from a subject. Alteration in the level, size or lack of cognate antigen would indicate a mutation. Antibodies specific for products of mutant alleles could also be used to detect mutant gene product. Such immunological assays can be done in any convenient format known in the art. These include Western blots, immunohistochemical assays and ELISA and RAPID assays.

The use of monoclonal antibodies in an immunoassay is particularly preferred because of the ability to produce them in large quantities and the homogeneity of the product. The preparation of hybridoma cell lines for monoclonal antibody production is derived by fusing an immortal cell line and lymphocytes sensitized against the immunogenic preparation (i.e. comprising a Bcl-x_{L}, Bak or Bax polypeptide) or can be done by techniques which are well known to those who are skilled in the art. (See, for example, Douillard et al., Basic Facts about Hybridomas, in Compendium of Immunology Vol. II, ed. by Schwartz, 1981; Kohler et al, Nature, 256:495-499, 1975; Kohler et al., European Journal of Immunology, 6:511-519, 1976).

In another aspect the present invention provides modified animals or cells for use inter alia in the development or testing of agents as described herein.

The genetically modified animals such as PLT20 or PLT16 mutants described herein and cells therefrom provide a sensitised system in which to study the affects of a range of apoptosis modifiers in the context of a depleted Bcl-2/Bak/Bax pathway.

Cells or constructs may be stored frozen and sold with instructions for use. In some embodiments, the modified animals are genetically modified, comprising mutations in pro-survival or proapoptotic genes, Bcl-x, Bak or Bax genes, such as partial loss of function mutations. The term “genetically modified” refers to changes at the genome level and refers herein to a cell or animal that contains within its genome one or more specific gene which have been altered. Alternations may be single base changes such as a point mutation or may comprise deletion of the enthe portions of the gene by techniques such as those using homologous recombination. Genetic modifications includes alterations to regulatory regions, insertions of further copies of endogenous or heterologous genes, insertions or substitutions with heterologous genes or genetic regions etc. Alterations include, therefore, single of multiple nucleic acid insertions, deletions, substitutions or combinations thereof resulting in partial loss of function of the gene.

Cells and animals which carry one or more modified allele/s can be used as model systems to study the effects of the gene products and/or to test for substances which have potential as therapeutic agents when these function are impaired. Animals for testing therapeutic agents can be selected after mutagenesis of whole animals or after treatment of germline cells or zygotes. After a test substance is applied to the cells, the phenotype of the cell is determined. Any trait of the cells can be assessed. Thus a genetically modified animal or cell includes animals or cells from a transgenic animal, a “knock in” or knock out” animal, conditional variants or other mutants or cells or animals susceptible to co-suppression, gene silencing or induction of RNAi.

Conveniently, targeting genetic constructs are initially used to generate the modified genetic sequences in the cell or organism. Targeting constructs generally but not exclusively modify a target sequence by homologous recombination. Alternatively, a modified genetic sequence may be introduced using artificial chromosomes. Targeting or other constructs are produced and introduced into target cells using methods well known in the art which are described in molecular biology laboratory manuals such as, for example, in Sambrook, Molecular Cloning: A Laboratory Manual, 3^{rd} Edition, CSHLP, CSH, NY, 2001; Ausubel (Ed) Current Protocols in Molecular Biology, 5^{th} Edition, John Wiley & Sons, Inc, NY, 2002.

Genetically modified organisms are generated using techniques well known in the art such as described in Hogan et al,, Manipulating the Mouse Embryo: A Laboratory Manual, Cold Spring Harbour Laboratory Press, CSH NY, 1986; Mansour et al., Nature, 336:348-352, 1988; Pickert, Transgenic Animal Technology: A Laboratory Handbook, Academic Press, San Diego, Calif., 1994. The present invention provides methods of generating genetically modified mice mutants comprising modifying the herein described mouse mutants using such techniques.

The present invention is further described by the following non-limiting Examples,

### Example 1

### Mutations in Bcl-x Cause Thrombocytopaenia

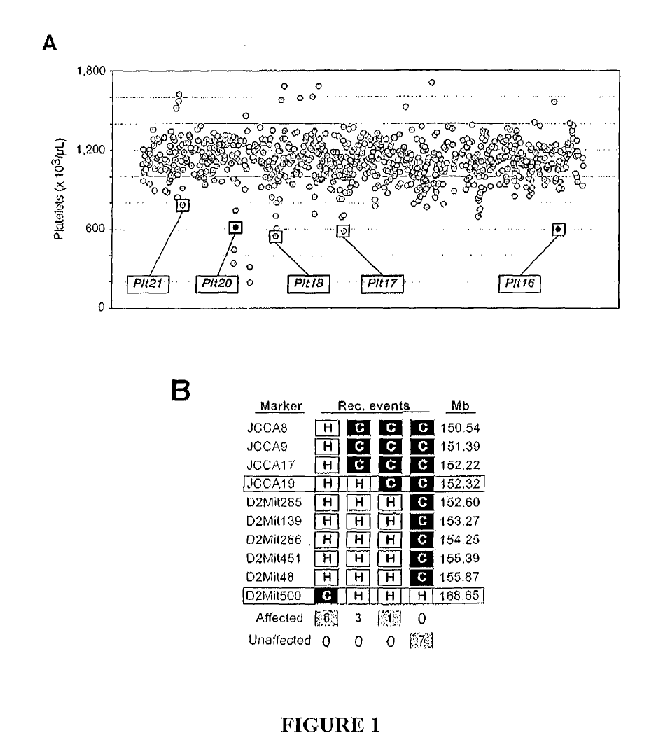

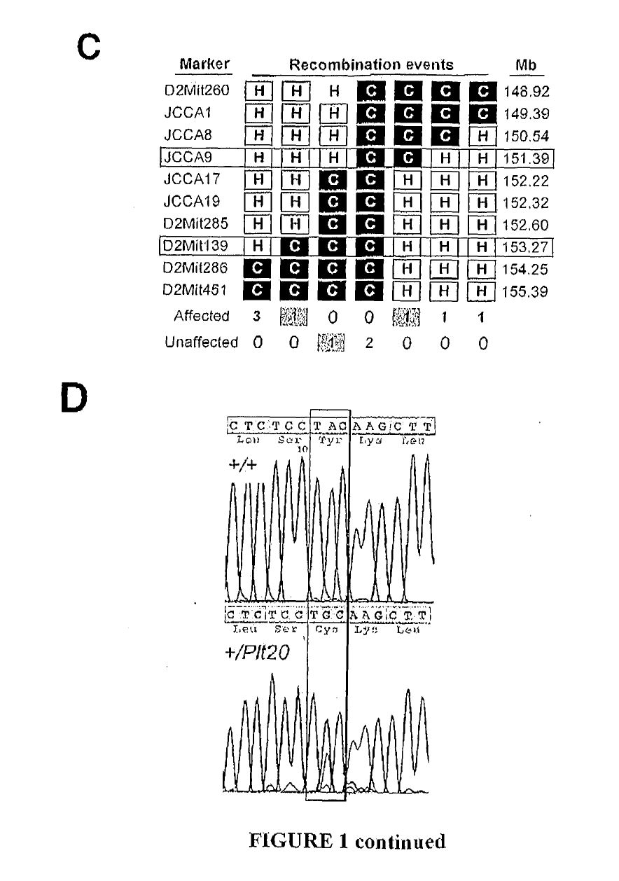

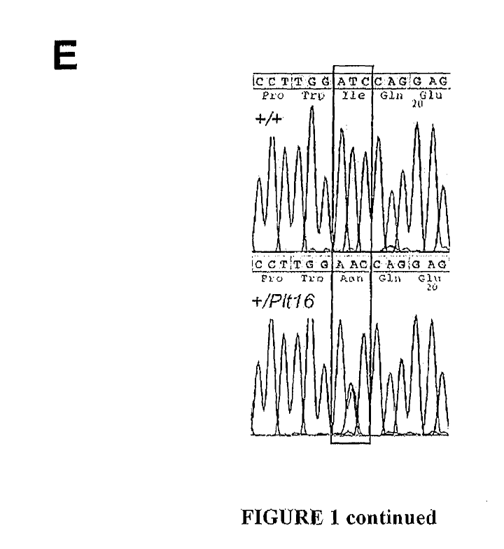

A genome-wide mutagenesis screen in wild type mice was conducted (Kile et al., Nat. Rev. Genet., 6:557-567, 2005) to identify mutations causing thrombocytopaenia. Male BALB/c mice were treated with the chemical mutagen N-ethyl-N-nitrosourea (ENU) and mated to untreated BALB/c females. First generation (G₁) offspring were bled at 7 weeks of age, and mice exhibiting circulating platelet counts below 900×10³/μL (lower end of the normal range) were re-bled at 9 weeks. Several G₁ mice exhibited persistent thrombocytopaenia (FIG. 1A), the heritability of which was in each case tested by mating to wild type BALB/c mice. The G₂ offspring from these matings were bled at 7 weeks of age, and the presence of animals with low platelet counts confirmed 5 pedigrees were segregating heritable dominant mutations causing thrombocytopaenia (˜600×10³/μL) (Table 6).

Two of these mutations, denoted Plt20 and Plt16, were both mapped, via a standard positional cloning approach, to the distal end of chromosome 2. Fine mapping refined the candidate regions for Plt20 (FIG. 1B) and Plt16 (FIG. 1C) to overlapping intervals of 16.3 and 1.9 Mb, respectively. The exons and splice junctions of candidate genes were directly sequenced, and mutations in the Bcl-x gene were identified in affected animals from both the Pit20 (FIG. 1D) and Plt16 (FIG. 1E) pedigrees. In the case of Plt20, an A-to-G transition is predicted to cause the substitution of cysteine for tyrosine at residue 15 of Bcl-x_{L}, the major protein encoded by the Bcl-x locus. In Plt16, the mutation is a T-to-A transversion predicted to cause the substitution of asparagine for isoleucine at residue 182.

### Example 2

### Like Bcl-x^{Plt20} and Bcl-x^{Plt16} Mice, Bcl-x_{L}-Deficient Mice are also Thrombocytopenic

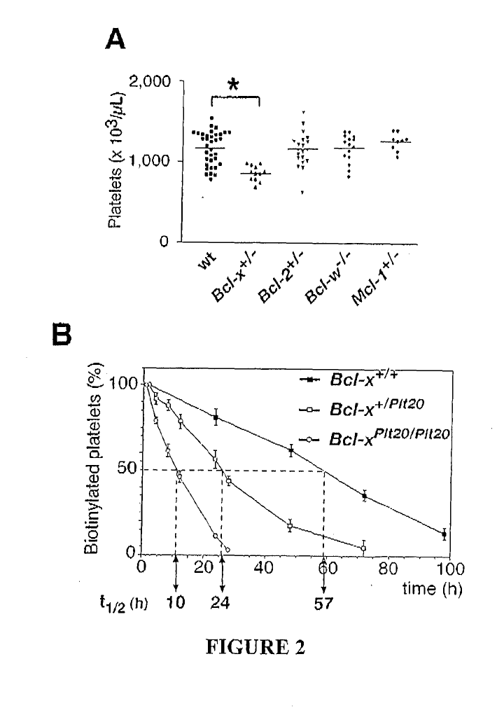

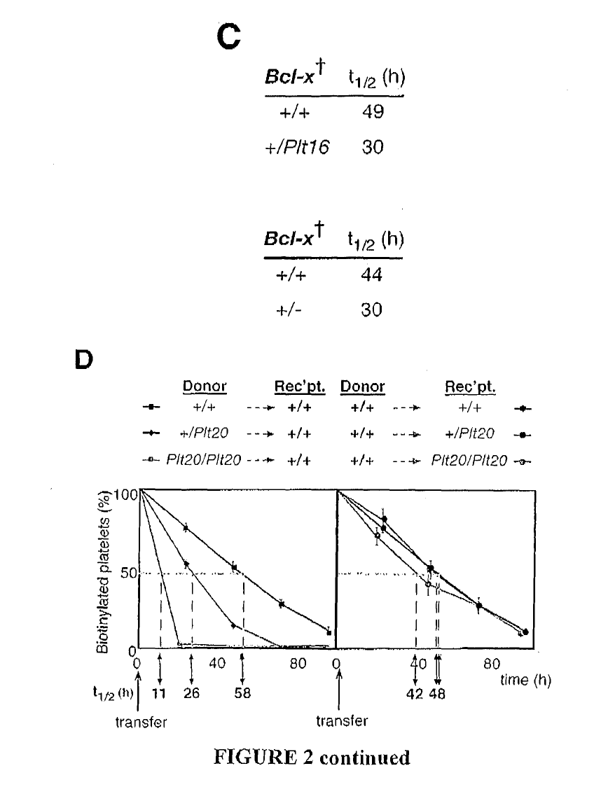

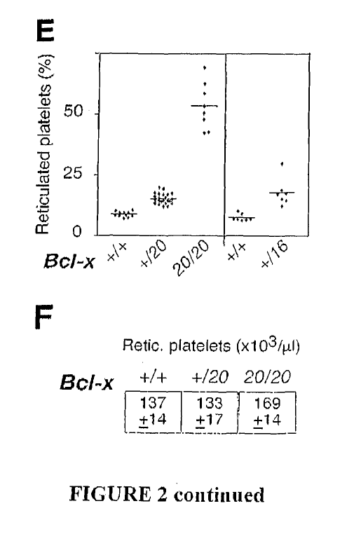

Bcl-x_{L} (Boise et al, 1993 (supra)) is a pro-survival member of the Bcl-2 protein family (which includes Bcl-2, Bcl-w, Mcl-1 and A1) that regulates developmentally programmed and stress induced cell death (Adams, 2003 (supra); Danial et al., 2004 (supra)). The thrombocytopaenia exhibited by mice carrying the Plt20 and Plt16 alleles of Bcl-x suggested that Bcl-x_{L} contributes to the maintenance of platelet numbers. To verify the role of Bcl-x_{L}, to preclude other Bcl-x gene defects (e.g. aberrant splicing, dominant activation) and to exclude linked ENU-induced mutations as the cause of the thrombocytopaenia, animals were examined that had been specifically engineered to lack Bcl-x_{L} (Motoyama et al., Science, 267:1506-1510, 1995). Bcl-x^{+/-} mice develop normally and are born at the expected Mendelian frequency (Motoyama et al, 1995 (supra)). Like Bcl-x^{+/Plt20} and Bcl-x^{+/Plt16} mice, Bcl-x^{+/-} animals exhibited platelet counts significantly lower (˜860×10³/μL) than wild type counterparts (˜1,100×10³/μL) (FIG. 2A), confirming that haploinsufficiency of Bcl-x results in thrombocytopaenia.

Unlike Bcl-x^{Plt16/Plt16} animals, of which only a few were observed at birth, and Bcl-x^{-/-} mice, which die at mid-gestation (Motoyama et al, 1995 (supra)), Bcl-x^{Plt20/Plt20} mice were born at the expected Mendelian frequency and survived to at least 6 months of age, indicating that this allele of Bcl-x is hypomorphic, rather than a complete loss-of-function. Aside from a mild increase in splenic erythropoiesis (data not shown), Bcl-x^{Plt20/Plt20} mice did not display any other gross abnormalities in the hematopoietic compartment (Table 6), and in contrast to animals carrying other alleles of Bcl-x (Kasai et al., Dev Biol 264;202-216, 2003), males were fertile, indicating that spermatogenesis is not significantly compromised. Significantly, platelet counts in homozygous Bcl-x^{Plt20/Plt20} mice were further reduced to approximately 25% that of their wild type littermates (Table 6), demonstrating that incremental reductions in Bcl-x_{L} produce a phenotypic gradient with respect to platelet number.

Thrombocytopaenia is not a general result of inactivating Bcl-2-like pro-survival proteins: unlike Bcl-x mutant mice, platelet counts in Bcl-2^{+/-}, Bcl-w^{-/-} and Mcl-1^{-/-} animals were normal (FIG. 2A).

### Example 3

### Increased Rates of Platelet Clearance in Bcl-x Mutant Mice

Robust megakaryocytopoiesis in the bone marrow and spleens of Bcl-x mutant mice argued against defective platelet production being the primary cause of thrombocytopaenia. Megakaryocyte progenitor numbers were normal in Bcl-x^{+/Plt20} mice and Bcl-x^{Plt20/Plt20} littermates (data not shown). Mature megakaryocytes were marginally increased in Bcl-x^{+/Plt20} and Bcl-x^{+/Plt16} mice, and significantly elevated in Bcl-x^{Plt20/Plt20} homozygotes (data not shown). In all Bcl-x mutant mice, they were morphologically normal and exhibited ploidy profiles similar to those of wild type counterparts (data not shown). Additionally, megakaryocyte progenitors from the mutant mice were not prone to spontaneous apoptosis in vitro (data not shown), and recovered as robustly as their wild type counterparts from stress-induced thrombocytopaenia in vivo (data not shown). These data indicate that mutations in Bcl-x do not significantly impair platelet production.

It was examined whether increased splenic sequestration might account for the reduction in platelet counts. However, removing spleens from homozygous Bcl-x^{Plt20/Plt20} mice only partially corrected the thrombocytopaenia (from 367±41 to 516±63×10³/μL, compared with 1,279±200 to 1,650±136×10³/μL in wild type littermates), indicating that abnormal splenic function is primarily not responsible for low platelet counts.

Next, it was examined whether Bcl-x_{L} might directly influence the fate of platelets, in its capacity as a pro-survival regulator. It was reasoned that platelets with reduced Bcl-x_{L} function might die prematurely and be removed from the blood at a faster rate compared to their wild type counterparts. By tracking the fate of platelets labeled in vivo with biotin (Ault et al., Exp. Hematol., 23:996-1001, 1992), it was determined that the Bcl-x^{Plt20} mutation decreased platelet half-lives in a dose-dependent manner (FIG. 2B). One mutant allele of Bcl-x reduced the normal platelet half-life (t_{1/2}) by approximately 50% (˜57 h to 24 h), whereas mutating both alleles triggered a further reduction in t_{1/2}, to less than 12 h (FIG. 2B). Similarly, platelets from Bcl-x^{+/Plt16} and Bcl-x^{+/-} mice also exhibited shortened platelet life spans (FIG. 2C).

To confirm that these changes in circulating half-life reflected properties intrinsic to platelets, reciprocal adoptive transfers were performed. Upon transfer into wild type recipients, Bcl-x^{+/Plt20} and Bcl-x^{Plt20/Plt20} platelets were cleared more quickly than wild type platelets (FIG. 2D), with half-lives indistinguishable from those seen in unmanipulated mice. Conversely, the clearance of wild type platelets transferred into Bcl-x^{+/+}, Bcl-x^{+/Plt20} or Bcl-x^{Plt20/Plt20} mice was identical, regardless of the recipient's genotype (FIG. 2D).

The age profile of circulating platelets was examined by staining with thiazole orange, a marker of young, RNA-replete, ‘reticulated’ platelets (Ault et al, 1992 (supra); Kienast et al, 1990 (supra)). The Plt20 and Plt16 mutations in Bcl-x caused dose-dependent increases in the proportion of positive cells (FIG. 2E), indicating that platelet populations in mice carrying these mutations were relatively younger. This is consistent with their overall life span being shortened. Absolute reticulated platelet numbers in Bcl-x^{+/Plt20} or Bcl-x^{Plt20/Plt20} mice were comparable to wild type mice (FIG. 2F), again supporting the conclusion that mutation of Bcl-x does not significantly impair platelet production (Table 6 and data not shown).

### Example 4

### Bcl-x^{Plt20} and Bcl-x^{Plt16} Mutations Destabilize Bcl-x_{L}

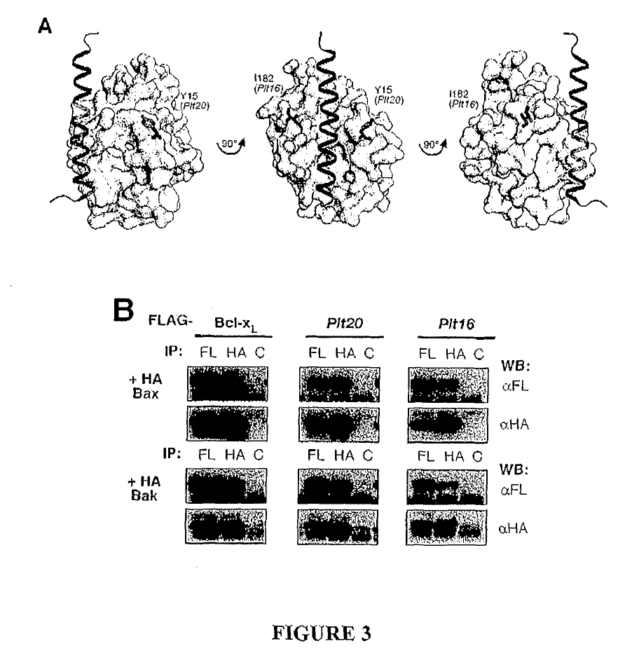

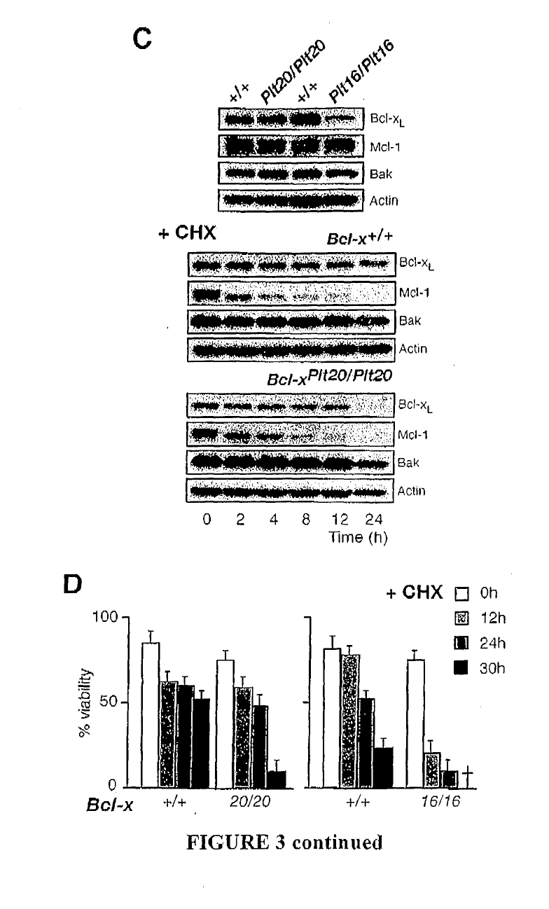

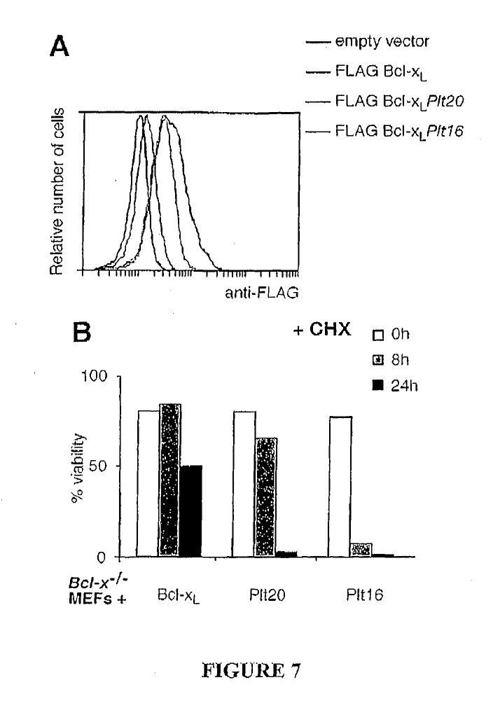

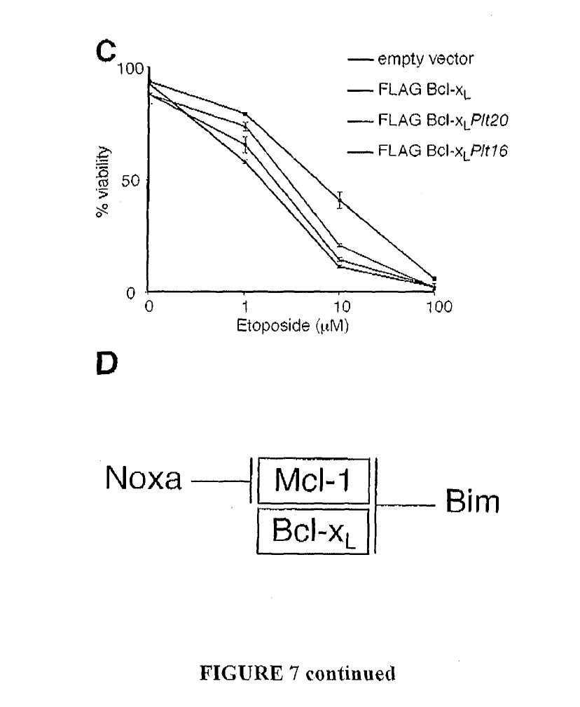

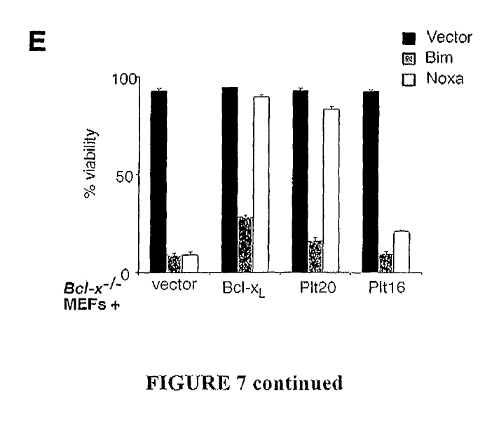

In contrast to Bcl-x^{-/-} mice, the absence of severe early developmental defects in homozygous Bcl-x^{Plt20/Plt20} mice argues there must be sufficient Bcl-x_{L} in most cell types to maintain adequate survival. Neither the Plt20 nor the Plt16 mutation directly affected the BH3 binding groove of Bcl-x_{L} (FIG. 3A), a region critical for its function (Adams, 2003 (supra); Liu et al., 2003 (supra); Sattler et al., Science, 275:983-986, 1997). Consistent with this, the capacity of the mutant Bcl-x_{L} proteins to bind the essential downstream mediators of apoptosis, Bax and Bak (Cheng et al., 2001 (supra); Lindsten et al., 2000 (supra)), appeared largely intact (FIG. 3B). When overexpressed in cell lines (including immortalized Bcl-x^{-/-} mouse embryo fibroblasts), the mutant Bcl-x_{L} proteins were not constitutively cytotoxic but instead were less stable that wild type Bcl-x_{L} (data not shown). Likewise, it was determined that the stability of endogenous full-length Bcl-x_{L} (normal t_{1/2}˜18 h) was moderately reduced in mouse embryo fibroblasts (MEFs) derived from Bcl-x^{plt20/plt20} mice (t_{1/2}˜12 h) (FIG. 3C). Even the basal level of endogenous Bcl-x_{L} was reduced in Bcl-x^{Plt16/Plt16} cells, consistent with the propensity of this mutant form of Bcl-x_{L} to be degraded (FIGS. 3C, 7A).

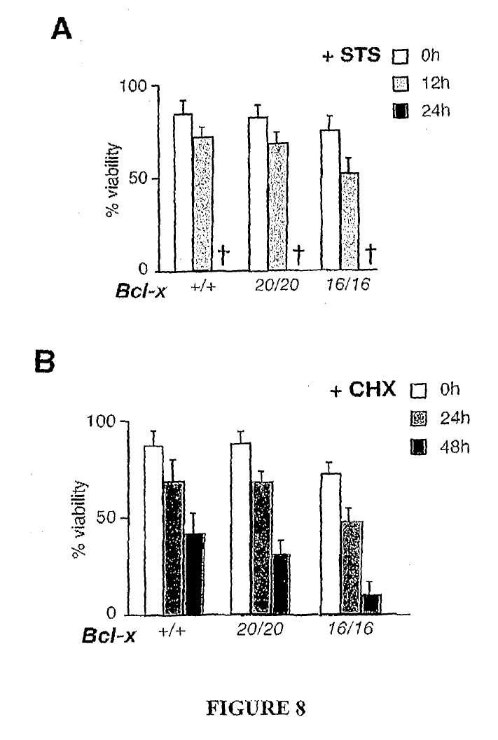

Interestingly, the destabilization of Bcl-x_{L} specifically sensitized MEFs to apoptosis when protein synthesis was inhibited (FIGS. 3D, 7B, 8B), because Bcl-x_{L} and Mcl-1, the two constraints on pro-apoptotic Bak (Willis et al., 2005 (supra)), were both degraded following cycloheximide treatment (FIG. 3C). In contrast, Bak was maintained even 24 h after this treatment. Because both pro-survival proteins (Bcl-x_{L} and Mcl-1) need to be inactivated for Bak-mediated apoptosis (Willis et al., 2005 (supra)), the greater stability of wild type Bcl-x_{L} allowed prolonged survival following cycloheximide treatment (FIGS. 3D, 7B) even when Mcl-1 was degraded (FIG. 3C). In contrast, the mutations had no effect on the sensitivity to other damaging signals that did not directly affect protein synthesis, such as treatment with the broad-spectrum kinase inhibitor staurosporine (FIG. 8A).

Thus, Bcl-x^{Plt20} and Bcl-x^{Plt16} are hypomorphic alleles of Bcl-x that encode labile proteins. The effect of these mutations is most marked when new protein synthesis is limited, potentially explaining why they produce such a striking phenotype in platelets, a cell type with a limited capacity to synthesize new proteins (Booyse et al. Biochim. Biophys. Acta., 157:660-663, 19681 Denis et al., Cell, 122:379-391, 2005; Kieffer et al., Eur. J. Biochem., 164:189-195, 1987; Weyrich et al, Proc. Natl. Acad. Sci. USA, 95:5556-5561, 1998).

### Example 5

### A BH3-mimetic Compound Causes Acute Thrombocytopaenia

If Bcl-x_{L} is required to maintain platelet survival in vivo, it was speculated that pharmacologically antagonizing its activity in wild type mice will trigger platelet death and result in thrombocytopaenia. The small molecule ABT-737 is a BH3 mimetic drug that antagonizes pro-survival Bcl-x_{L} (Oltersdorf et al., 2005 (supra)). It selectively targets Bcl-2, Bcl-x_{L} and Bcl-w but not the other pro-survival proteins Mcl-1 or A1 (Oltersdorf et al., 2005 (supra); van Delft et al., Cancer Cell, 10:389-399, 2006). To date, it is reported to be well-tolerated in mice and demonstrates single agent efficacy against certain tumor cell lines, particularly those derived from small cell lung cancers or lymphomas (Oitersdorf et al., 2005 (supra)).

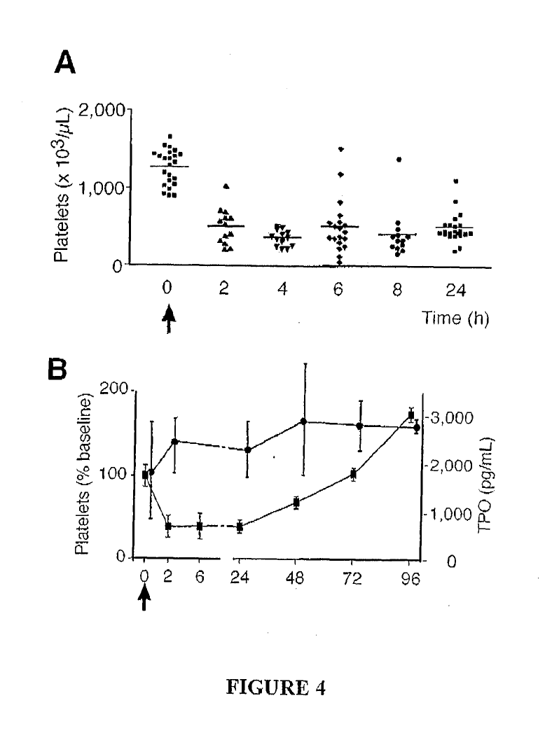

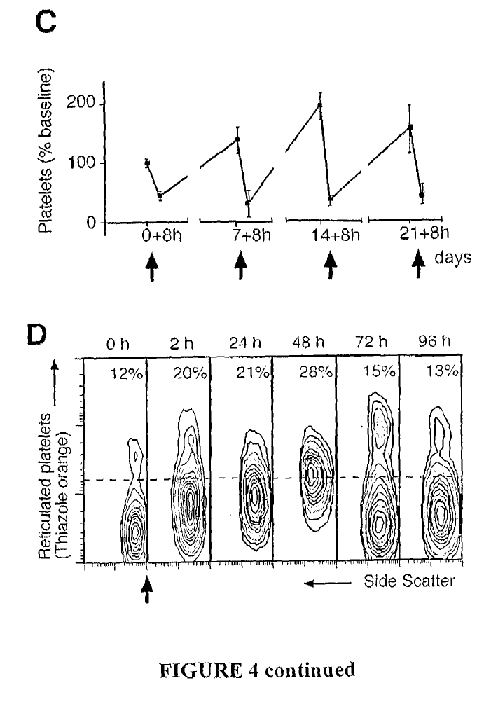

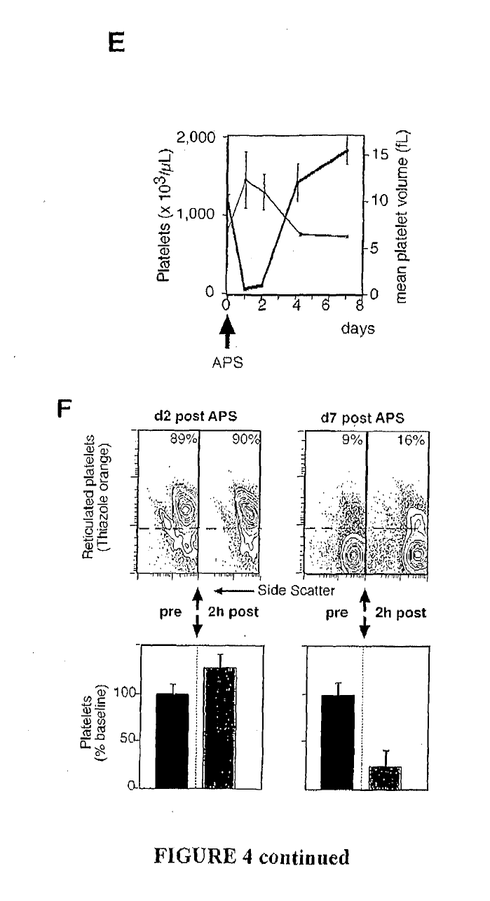

Within two hours of injecting a single dose of ABT-737 (but not the vehicle control) into wild type C57BL/6 mice, platelet counts dropped to less than 30% of normal, with the nadir at 4 hours (FIG. 4A and data not shown). Thrombocytopaenia was dose dependent (data not shown) and platelet counts gradually recovered to reach normal levels by 3 days post-treatment (FIG. 4B). This recovery, associated with sustained production of thrombopoietin (TPO) (FIG. 4B), was also observed even with daily injections of ABT-737. When ABT-737 was continued for 14 days, platelet levels were maintained at ˜60-70% of normal until the therapy was stopped (data not shown). In contrast, when ABT-737 was given weekly, acute thrombocytopaenia ensued in a cyclical manner (FIG. 4C), with platelet counts recovering in the intervening periods.

Interestingly, when the age profile of circulating platelets post ABT-737 was examined by thiazole orange staining, a transient increase in the proportion of reticulated platelets was noted (FIG. 4D). This suggested that older platelets might be more susceptible to the effects of the drug. To investigate this possibility, mice were treated with anti-platelet serum (APS) in order to artificially synchronize platelet production. Platelet counts post APS decreased rapidly to almost undetectable levels at 24 hours, before recovering over the course of 7 days (FIG. 4E). During this recovery, the thiazole orange profile changed dramatically, with a largely homogenous population of new, reticulated platelets prominent on Day 2 after APS (FIG. 4F). As the population aged, the proportion of reticulated platelets dropped to near-normal levels at 7 days post APS. Newly synthesized young platelets (2 days post-APS) were highly resistant to the effects of ABT-737 (FIG. 4F). In sharp contrast, aged platelets (7 days post APS) were susceptible to ABT-737 in vivo, confirming that the drug acts primarily on older platelets.

Drugs are a well-known cause of thrombocytopaenia. Aside from bone marrow suppression caused by cytotoxic agents, this is usually immune mediated. Typically, in such cases, onset of thrombocytopaenia occurs 7-10 days after initial exposure to a drug and is inevitably exacerbated by continued treatment (George et al., Ann. Intern. Med., 129:886-890, 1998). Conversely, the effect of ABT-737 was extremely rapid (FIG. 4A) and platelet counts in the treated mice partially recovered despite ongoing therapy (data not shown), suggesting that a new rheostat for maintaining platelet levels had been set. It was observed that TPO levels (FIG. 4B) and megakaryocytopoiesis were normal, or probably even elevated, in treated mice (data not shown). Furthermore, when ABT-737 was tested in progenitor cell cultures in vitro, no effect on the number of megakaryocyte colonies formed was evident (data not shown).

Thus, in contrast to the well-characterized effects observed when drugs impair platelet production or trigger immune-mediated destruction, the results described herein point towards a direct cytotoxic action of ABT-737 as an example of a pro-apoptotic molecule on platelets.

### Example 6

### ABT-737 Induces Caspase-Dependent Platelet Death

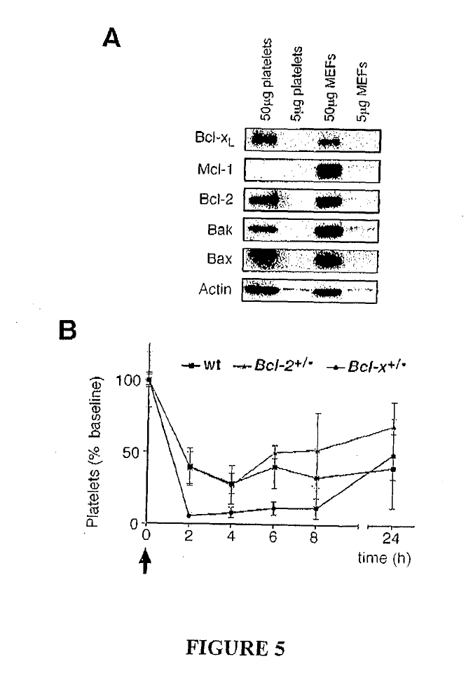

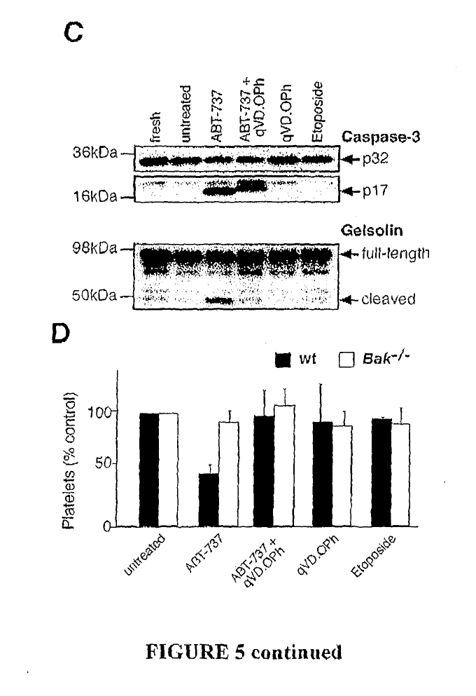

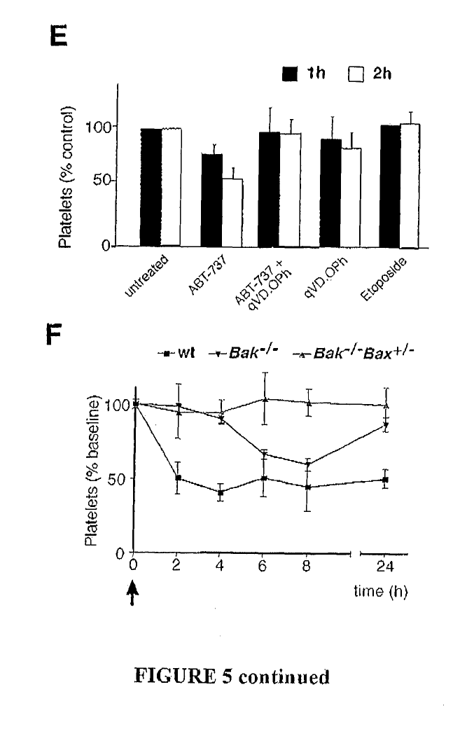

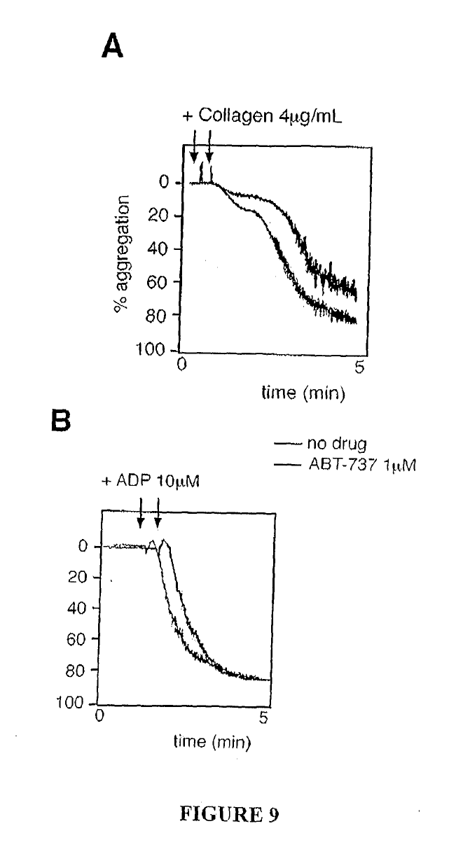

As anticipated, heterozygosity for a null allele of Bcl-x_{L}, but not Bcl-2, exacerbated ABT-737-induced thrombocytopaenia (FIG. 5B). Exposure to ABT-737 triggered cleavage and full activation of caspase-3 as well as cleavage of gelsolin, a known caspase substrate, in cultured platelets (FIG. 5C). Furthermore, inhibiting caspases, the downstream effectors of apoptosis (Thornberry et al., Science, 281:1312-1316, 1998), with the broad-spectrum inhibitor qVD.OPh (Caserta et al., Apoptosis, 8:345-352, 2003) partially ameliorated the cytotoxic effect of ABT-737 on both mouse (FIG. 5D) and human (FIG. 5E) platelets in culture. Of note, exposure to ABT-737 ex vivo did not trigger platelet activation or adversely impact upon the ability of platelets to aggregate in response to ADP or collagen (FIG. 9).

### Example 7

### Loss of Bak Ameliorates Thrombocytopaenia Caused by ABT-737

To explore how antagonism of Bcl-x_{L} might trigger platelet destruction by caspases, the potential molecular target(s) of its activity were considered. The likeliest candidates are the multi-domain pro-apoptotic family members, Bax and Bak, since they have been shown to be essential mediators of apoptotic cell death (Cheng et al., 2001 (supra); Lindsten et al., 2000 (supra); Rathmell et al., 2002 (supra); Zong et al., 2001 (supra)) that act upstream of the caspases. Furthermore, Bcl-x_{L} has the capacity to keep Bak in check by directly binding this cell death mediator (FIG. 3B; see discussion above) (Willis et al., 2005 (supra)), thereby preventing its downstream actions. The effect of ABT-737 in mice deficient for either one or both of these proteins was therefore examined. The absence of Bak markedly blunted the action of ABT-737 on platelet viability in culture (FIG. 5D) and significantly buffered against the apoptotic effects of ABT-737 in vivo (FIG. 5F). While the loss of Bax alone had little impact (data not shown), the complete absence of Bak combined with the additional loss of one Bax allele rendered platelets enthely refractory to ABT-737 (FIG. 5F).

These data show that ABT-737 as an example of a Bcl-x_{L} antagonist actively induces the killing of platelets by neutralizing the pro-survival action of Bcl-x_{L}, thereby allowing the unrestrained action of the key pro-apoptotic mediators Bak, and to a lesser extent Bax.

### Example 8

### In Platelets, Pro-Apoptotic Bak is the Critical Target for Pro-Survival Bcl-x_{L}

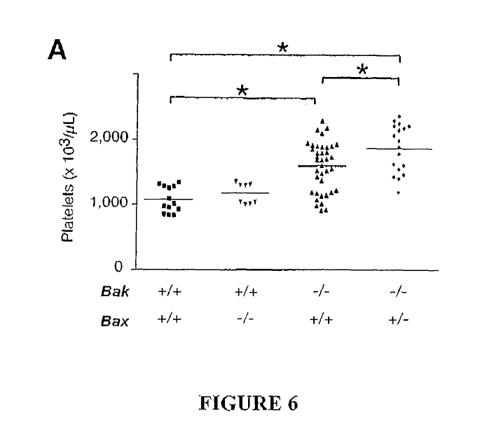

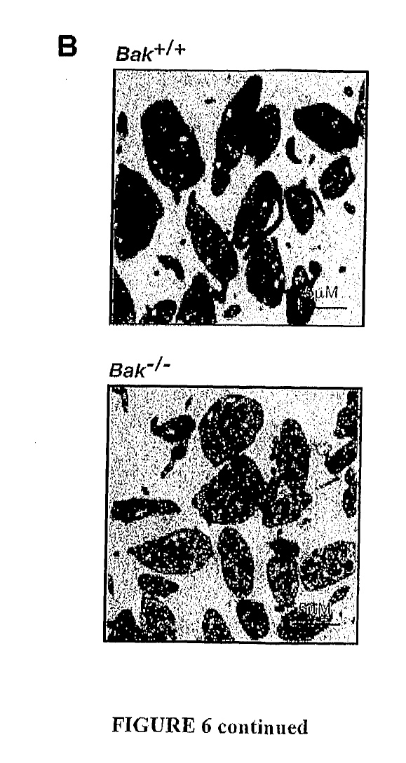

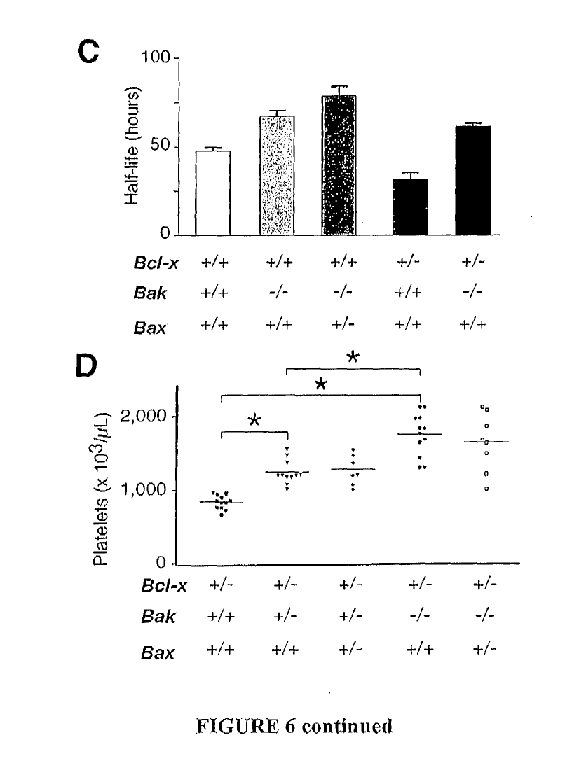

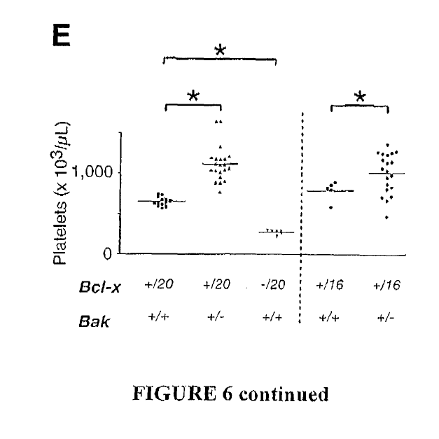

Since deletion of the downstream effectors Bak and Bax protected platelets against ABT-737-induced killing the role of these molecules in normal platelet homeostasis was examined. At steady state, platelet counts in Bax-deficient mice were indistinguishable from those of wild type counterparts (FIG. 6A). In contrast, Bak^{-/-} mice exhibited a marked increase in platelet numbers (FIG. 6A). Bak^{-/-} platelets were morphologically normal (FIG. 6B) and Bak^{-/-} mice do not have defects (e.g. hyposplenism) that might account for the thrombocytosis. It was therefore of interest to find that platelet half-life in these animals, as determined by in vivo labeling assays, was increased by almost 50% (FIG. 6C and data not shown). Platelet counts and half-lives were examined in mice carrying combinations of mutated Bcl-x, Bak and/or Bax alleles. Strikingly, the loss of one Bak allele rescued the thrombocytopaenia in Bcl-x^{+/-} mice (FIG. 6D). Even with reduced Bcl-x_{L} in Bcl-^{+/-} mice, the absence of both Bak alleles prolonged platelet half-lives (FIG. 6C) and caused a thrombocytosis similar to that seen in the Bak-deficient animals (c.f. FIG. 6D with 6A). Furthermore, the thrombocytopaenia seen in heterozygous Bcl-x^{Plt20} or Bcl-x^{Plt16} mice was exacerbated by the constitutive absence of the other Bcl-x allele and reversed by loss of one Bak allele (FIG. 6E). Thus, Bak lies biochemically (Willis et al, 2005 (supra)) (FIGS. 3, 5) and genetically (FIG. 6) downstream of Bcl-x.

### Example 9

### Cellular Screens for Compounds that Maintain Cell Viability

Platelets are not readily amenable to manipulation ex vivo. Accordingly, in order to identify compounds (agents) that promote cellular survival (including enhanced life span/viability) cells are used that are sensitive to apoptosis inducing agents. For example, cells in which Bcl-x_{L} is the key control on Bak are used to screen for small molecules or other compounds (agents) that inhibit cell death, even when Bcl-x_{L} is inactivated. Such compounds are useful for maintaining cell viability. In one example, mouse embryo fibroblasts (MEFs) are engineered to lack Mcl-1, which is the other control on Bak (Willis et al., 2005 (supra)). Inactivating Bcl-x_{L} with, for example, ABT-737 does not cause cell death unless Mcl-1 is also inactivated (van Delft et al., 2006 (supra)). In the constitutive absence of Mcl-1, MEFs are highly sensitive to such as ABT-737. In this situation, the only brake on Bak is Bcl-x_{L}, which can be abrogated by a compound ABT-737 that acts as a potent inhibitor of Bcl-x_{L}. Accordingly, compound libraries are screened for those compounds that can block ABT-737-induced killing of Mcl-1 deficient MEFs. Such compounds may act to directly block ABT-737 or the action of the cell death mediators Bak or Bax, or Bak and Bax.

In one non-limiting example, Mcl-1-null MEFs are plated onto flat-bottomed 96-well plates. 12-24 h later, a library compound is added at 0.1, 1 and 10 μM final concentration and incubated for 2 h followed by addition of ABT-737 (100 nM) or a carrier vehicle. Cell viability is scored 24 h later vising Alamar Blue dye and read 4 h later. The cell viability in the absence of either library compound or ABT-737 acts as a positive control. Lack of cell viability in the presence of no library compound and ABT-737 acts as a negative control. Inert compounds show normal cell viability in the absence of ABT. Cytotoxic compounds show reduced cell viability in the presence of library compounds but absence of ABT-737. Positive hits show increased cell viability in the presence of ABT-737 and a library compound, while negative compounds show reduced cell viability in the presence of a library compound and ABT-737. Positive hits are tested on several independent cell lines and on platelets in culture. In some embodiments, the compounds may act by blocking cellular uptake of ABT-737 or inhibiting the action of ABT-737 in cells. In other embodiments, the compounds act by directly inhibiting Bak, Bax or both Bak and Bax or indirectly inhibiting these molecules or apoptosis effector molecules downstream of Bak or Bax. The methods are also practised on modified mice with Bcl-2-family genes modified in order to further sensitise the screen and detect the molecular targets of the each positive agent.

### Example 10

### Supplemental Experimental Procedures

### Generation and Isolation of Bcl-x^{Plt30} and Bcl-x^{Plt16}

Male BALB/c mice were injected intra-peritoneally with three 100 mg/kg doses of ENU (Nitroso-N-ethylurea, Sigma N3385 1 g isopac) given at weekly intervals. Treated mice were allowed to recover fertility for 8 weeks before mating with untreated BALB/c females to yield first-generation (G₁) progeny. At 7 weeks of age, blood from G₁ mice was collected from the retroorbital plexus into tubes containing potassium EDTA (Sarstedt) and the number of platelets in the peripheral blood was determined using an Advia 120 automated haemtological analyzer (Bayer),

### Genetic Mapping

The Plt20 and Plt16 mutations were mapped by outcrossing affected animals to wild type C57BL/6 mice. The F₁ offspring from these matings were screened for platelet number, and affected F₁ mice were then outcrossed to wild type C57BL/6 mice to produce the F₂ generation. Genomic DNA was collected from 40 F₂ animals in each case and a genome-wide scan performed with a panel of 80 simple sequence length polymorphism (SSLP) markers. Candidate intervals were refined by analysing the products of additional meioses with MIT and in-house CA repeat markers at increasing density.

### Nucleic Acid Sequencing

Genomic DNA was extracted from tail biopsies and subjected to PCR amplification. To remove primers and unincorporated nucleotides, post-PCR reactions were treated with ExoSAP-IT (USB) according to the manufacturer's instructions and filtered through Sephadex columns. Amplicons were then sequenced directly using BigDye Terminator v3.0 (Applied Biosystems).

### Haemtological Analyses

The hematocrit, platelet and white cell count, and differential were determined by using either manual or automated (Advia 120) counting techniques. In most experiments, with the exception of data shown in FIGS. 1A and 6E, the data was collected from male mice. Clonal cultures of 2.5×10⁴ adult bone marrow cells or 5×10⁴ spleen cells in 1 mL of 0.3% agar in DMEM supplemented with newborn calf serum (20%) were stimulated with a mixture of 100 ng/mL murine stem cell factor, 10 ng/mL murine IL-3, 4 units/mL human EPO and incubated for 7 days at 37° C. in a fully humidified atmosphere of 10% CO₂ in air. Agar cultures were fixed, sequentially stained for acetylcholinesterase, Luxol fast blue and hematoxylin; the cellular composition of each colony determined at ×100-400 magnification. Megakaryocyte counts were performed by manual counting from sections of sternum and spleen after staining with hematoxylin/eosin. A minimum of 10 high-power fields (×200) were scored.

### Platelet Clearance Analysis

Mice were injected intravenously with 600 μg N-hydroxysuccinimido-biotin (NHS-biotin) (Sigma) in buffer containing 140 mM NaCl and 10% DMSO. At various times points whole tail blood was isolated and mixed with BSGC buffer (116 mM NaCl, 13.6 mM tri-sodium citrate, 8.6 mM Na₂HPO₄, 1.6 mM KH₂PO₄, 0.9 mM EDTA, 11.1 mM glucose). The equivalent of 1 μL blood was washed in balanced salt solution (BSS: 149 mM NaCl, 3.7 mM KCl, 2.5 mM CaCl₂, 1.2 mM MgSO₄, 7.4 mM HEPES, 1.2 mM KH₂PO₄, 0.8 mM K₂HPO₄, 3% bovine calf scrum), pelleted at 1,210 g for 10 min and stained with FITC conjugated rat anti-CD41 (BD) and phycoerythrin-conjugated streptavidin (BD) for 1 h on ice. 50,000 allophycocyanin-conjugated beads (Flow Cytometry Standards) were added per sample to facilitate quantitation of absolute platelet number. Samples were washed again in BSS and flow cytometry was performed on an LSR flow cytometer (BD). Platelets were distinguished from other cells by size and CD41 staining; biotinylated platelets were phycoerythrin positive.

### Adoptive Platelet Transfer

Mice were injected intravenously with 600 mg N-hydroxysuccinimido-biotin (NHS-biotin) (Sigma) in buffer containing 140 mM NaCl and 10% DMSO, 30 minutes after biotin injection tire mice were heart bled using a heparinized syringe and 2 mL blood mixed with 5 mL BSGC buffer. Blood was centrifuged twice at 600 g for 3 min and 5 mL platelet rich plasma was removed each time. Pooled platelet rich plasma was pelleted at 1210 g for 10 min and resuspended in 154 mM NaCl before intravenous injection into recipient mice. At various times post injection blood was isolated from recipient mice and analyzed as described above.

### Reticulated Platelet Labeling

Staining of reticulated platelets was performed using thiazole orange. Reactions contained 1 μL blood, 50 μL thiazole orange (0.1 μg/mL in PBS), 0.25 μL phycoerythrin conjugated CD41 antibody (BD) and 9 μL PBS. Reactions were incubated in the dark at room temperature for 15 min before being fixed by the addition of 1 mL of PBS 1% PFA. Platelets were distinguished from other cells on the basis of size and CD41 expression.

### Anti-Platelet Serum

For each mouse, 200 μL rabbit anti-mouse platelet serum (APS; Intercell Lot number 6309) was injected intra-peritoneally at 1:60 dilution.

### Animal Experimentation

All animal experiments conformed to the regulatory standards of, and were approved by, the Melbourne Health Research Directorate Animal Ethics Committee, Mice lacking Bcl-x_{L} (Motoyama et al, 1995 (supra)), Bcl-2 (Veis et al., Cell, 75:229-240, 1993), Bcl-w (Print et al., Proc. Natl. Acad. Sci. USA, 95:12424-12431, 1998), Bax (Knudson et al., Science, 270:96-99, 1995) and Bak (Lindsten et al., 2000 (supra)) have been previously described. They were originally generated on a mixed C57BL/6×129Sv background (using 129Sv ES cells for gene targeting). These strains have been backcrossed for at least 10 generations with C57BL/6 mice before use in this study. Mice lacking Mcl-1 were generated on an inbred C57BL/6 background and will be fully described elsewhere (PNK, A Strasser and P Bouillet, manuscript in preparation). Briefly, tire Mcl-1 targeting construct was prepared from C57BL/6 (clone 75A5) BAC DNA and the 4.6 kb Mcl-1 coding region was flanked by loxP sites electroporated into C57BL/6 ES cells, and injected into 129 blastocysts. After germline transmission had been established, chimeric black offspring were crossed with C57BL/6 transgenic mice expressing Cre recombinase to obtain Mcl-1^{+/-} mice; successful excision was confirmed by PCR genotyping.

The Plt20 and Plt16 mutations were induced on an inbred BALB/c background and backcrossed to C57BL/6. All Bcl-x^{Plt20} and Bcl-x^{Plt16} mice used in this study were on a mixed background, having been backcrossed at least 2, but not more than 6, generations with C57BL/6 mice. Although no significant differences in platelet number were observed between the parental C57BL/6 and BALB/c strains, consistent with previous reports (Kile et al., Mamm. Genome, 14:81-85, 2003; Peters et al., Physiol. Genomics., 11:185-193, 2002, littermates were used as controls as appropriate.

### Platelet Aggregometry

Whole blood was collected in sodium citrate (3.2%) and platelet rich plasma obtained by centrifugation at 800 rpm for 12 min at room temperature. Platelet poor plasma was then obtained by centrifugation of remaining blood at 3,000 rpm for 15 min at room temperature. Platelet count was adjusted to 250×10⁹/L using platelet poor plasma. Diluted platelet rich plasma was placed in an aggregometer cuvette warmed to 37° C. and stirred whilst light transmission through the plasma was measured in a 4 channel platelet aggregometer. After baseline measurements were obtained, ADP or Collagen (both from Edward Keller) was added to induce aggregation.

### Thrombopoietin Analysis

Blood was collected from the retroorbital sinus using a non-heparinized capillary tube into an eppendorf tube and incubated at room temperature for 2 h. After centrifugation at 5,500 rpm for 20 min, the supernatant was centrifuged again under the same conditions and serum collected. Thrombopoietin levels were measured by enzyme-linked immunosorbent assay (ELISA) using the Quantikine Mouse TPO Immunoassay kit (R&D Systems) according to the manufacturer's instructions.

### Flow Cytometric Analysis of Megakaryocytes

Bone marrow from femurs was flushed into 2 mL of CATCH medium (0.38% sodium citrate, 1 mM adenosine, 2 mM theophylline, 3 μg/mL prostaglandin h in calcium- and magnesium-free HBSS) and dispersed gently with a transfer pipette. The cell suspension was filtered through a 100 μm cell strainer to remove cell clumps and debris, and centrifuged at 400 g for 4 min at RT. After resuspension in 1 mL of CATCH diluted 1:1 with PBS containing 5% FBS, non-specific binding of antibodies was blocked by pre-incubation of cells with an antibody to CD16 and CD32. Cells were labeled with FITC-conjugated anti-CD41 antibody on ice for 30 min, and washed with 4 mL of CATCH 1:1 with PBS containing 5% FBS. After centrifugation at 400 g for 4 min at RT, cells were resuspended in 1 mL hypotonic propidium iodide (50 μg/mL in 0.1% sodium citrate), and incubated on ice for 15 min. RNAse A was then added to a final concentration of 50 μg/mL, and after incubation at RT for 30 min, the cells were filtered through a 100 μm cell strainer and analyzed.

### Electron Microscopy

Platelet-rich plasma was diluted in, and bone marrow from femurs was flushed into, 10 volumes of 2.5% glutaraldehyde, 2.0% formaldehyde in 0.1 M cacodylate buffer with 2 mM CaCl. After fixation for 1.5 h, samples were washed twice with 0.1 M cacodylate buffer, and post-fixed in 1% osmium tetroxide/0.1 M cacodylate buffer for 1 h. Samples were buffer washed, dehydrated through a graded series of ethanol concentrations, subjected to intermediate dehydration with propylene oxide, then gradual resin infiltration with Glauert EMBED resin mix to propylene oxide followed by infiltration with pure resin. Samples were then placed in BEEM capsules with pure resin and cured at 60° C. for 2 days. Sections of 500 μm thickness were cut and counterstained with toluidine blue. Subsequently, thin sections, of 60 nm were cut and stained with 1% aqueous uranyl acetate, and Reynold's lead citrate. Grids were viewed using a Hitachi-H7500 transmission electron microscope. Digital images were acquired by a Gatan 2k×2k CCD camera.

### Expression Constructs

Mammalian expression vectors for FLAG-tagged wild-type or mutant mouse Bcl-x_{L}, and HA-tagged human Bax or Bak were sub-cloned into pEF PGKpuro or pEF PGKhygro as described previously (Chen et al., 2005 (supra); Huang et al., EMBO. J., 16:4628-4638, 1997; O'Connor et al., EMBO. J., 17:384-395, 1998). Retroviral expression constructs for human Bim_{S} and human Noxa were made by sub-cloning into the pMIG vector (Chen et al., 2005 (supra)), and for wild-type or mutant, mouse Bcl-x_{L} into the pMIH vector (MSCV-IRES-hygromycin) (van Delft et al., 2006 (supra)). All constructs were verified by sequencing and details of all oligonucleotides and constructs are available from die authors.

### Tissue Culture, Cell Death Induction, Retroviral Infections and Apoptosis Assays

Cell lines (HEK293T: immortalized human embryonal kidney cell line, Phoenix Ecotropic packaging cells and mouse embryonic fibroblasts: ‘MEFs’) were cultured in Dulbecco's Modified Eagles (DME) medium supplemented with 10% fetal calf serum (FCS), and in some cases with 250 μM L-asparagine and 50 μM 2-mercaptoethanol. All MEFs were generated from E13-14.5 embryos as previously described (Chen et al., 2005 (supra); Willis et al., 2005 (supra)). IL-3 dependent (factor-dependent myeloid-FDM) cell lines were generated by co-culturing E14.5 fetal liver single cell suspensions with fibroblasts expressing a HoxB8 retrovirus in the presence of high levels of IL-3, as previously described (Ekert et al., 2004 (supra)). Cells expressing Bcl-x_{L} or its mutants were generated by retrovirally infecting the cells with pMIH retroviruses (Chen et al., 2005 (supra)). Retroviral constructs were transiently transfected into Phoenix Ecotropic packaging cells and viral supernatants were used to infect cells as described (Chen et al., 2005 (supra)). Pools of expressing cells were selected by addition of 1 mg/mL hygromycin.

Cell death was induced with ABT-737 (up to 5 μM, Abbott Laboratories (Oltersdorf et al., 2005 (supra)), etoposide (up to 100 μM, Pharmacia) or staurosporine (10 μM, Sigma); 50 μM qVD.OPh (Enzyme Systems) was also added in some experiments. Cell viability was determined by flow cytometry for cells that exclude propidium iodide or manual counting using a hemacytometer.

### Immunoprecipitation, Immunoblotting and Immunostaining

Cell lysates were prepared in lysis buffer (20 mM Tris-pH 7.4, 135 mM NaCl, 1.5 mM MgCl₂, 1 mM EGTA, 10% glycerol) containing 1% Triton X-100 (TX-100), supplemented with protease inhibitors (Roche and Sigma). Immunoprecipitation was performed as described (Chen et al., 2005 (supra); Huang et al., 1997 (supra); O'Connor et al., 1998 (supra)) using mouse monoclonal antibodies to FLAG (M2: Sigma) or HA (HA.11; CRP); control immunoprecipitations were performed using mouse anti-Glu-Glu (MMS-115R: CRP). Proteins were resolved by SDS:PAGE (Novex gels: Invitrogen), transferred onto nitrocellulose membranes and detected by immunoblotting using rat monoclonal anti-HA (3F10: Roche) and anti-FLAG (9H1, (Wilson-Annan et al., J. Cell. Biol., 162:877-888, 2003) antibodies.

Other antibodies for immunoblotting were mouse monoclonal anti-Bcl-x_{L} (2H12; BD), anti-Bcl-2 (clone 7; BD), anti-human Bcl-2 (Bcl-2-100), anti-actin (AC40; Sigma), anti-Bax (2D2 and 5B7; Sigma), anti-Mcl-1 (clone 22: BD); rat monoclonal anti-Mcl-1 (14C11; D Huang, unpublished), rabbit polyclonal anti-Bak (B5897: Sigma), anti-Mcl-1 (Rockland), anti-caspase-3 (585; gift of Y. Lazebnik), anti-cleaved p17 fragment of caspase-3 (AB3623; Chemicon), anti-gelsolin (gift of D Kwiatkowski) (Kothakota et al., Science, 278:294-298, 1997); hamster monoclonal anti-mouse Bcl-2 (3F11; BD). Secondary antibodies included HRP-conjugated anti-rat or anti-hamster IgG (SouthernBiotech), anti-mouse or anti-rabbit IgG (Chemicon), and anti-mouse IgG Fcγ chain specific antibody (Jackson ImmunoResearch). The proteins were detected using Enhanced ChemiLuminescence (ECL; GE Healthcare).

Expression of FLAG-tagged Bcl-x_{L} was also determined by immunostaining using the mouse monoclonal anti-FLAG M2 antibody before incubating with FITC-conjugated goat-anti-mouse IgG (10 μg/mL; SouthernBiotech); the samples were analyzed using a FACScan® (BD).

### ABT-737 Administration

Mice were injected intra-peritoneally with 75 mg/kg of ABT-737 given as a single dose or daily. A stock solution of ABT-737 (1 g/mL in DMSO) was diluted in a mixture of 30% propylene glycol, 5% Tween 80, 65% D5W (5% dextrose in water), pH 4-5; the final concentration of DMSO was less than 1%.

Ex vivo Platelet Assays

Blood was collected from indicated mice using a heparinized syringe and diluted 1:5 into buffered saline glucose, citrate with 1 mg/mL of prostaglandin I₂. Human whole blood was obtained with informed consent (approved by the Institutional Review Board), Platelet rich plasma was obtained and incubated at room temperature with drug or control for up to 2 h. Platelets were counted manually in triplicate using a hemacytometer.

### Example 11

### Discussion

These results presented here identify Bcl-x_{L} as the major homeostatic regulator of platelet survival. In contrast, the other Bcl-2-like pro-survival proteins do not play a significant role. Genetic or pharmacological antagonism in vivo or in vitro of Bcl-x_{L} caused a dose-dependent diminution of platelet survival and life span, but mutations in Bcl-2, Bcl-w or Mcl-1 did not. Furthermore, a role for pro-survival Mcl-1 or A1 in vivo is not contemplated since the BH3 mimetic compound ABT-737 does not target these members of the pro-survival Bcl-2 family (Oltersdorf et al., 2005 (supra); van Delft et al., 2006 (supra)).

The unique role Bcl-x_{L} plays in the maintenance of platelet survival is distinct from its proposed involvement in platelet formation. Overexpression of Bcl-2 in the hematopoietic compartment causes thrombocytopaenia (Ogilvy et al., 1999 (supra)), as does deletion of the gene encoding the pro-apoptotic BH3-only protein Bim (Bouillet et al., 1999 (supra)). Furthermore, overexpression of Bcl-x_{L} or Bcl-2 in megakaryocytes impairs pro-platelet formation (De Botton et al., Blood 100:1310-1317, 2002; Kaluzhny et al., 2002 (supra)). Together with evidence that activated caspases are required for platelet shedding, these data have implicated the apoptotic machinery in the massive ‘para-apoptotic’ (Thiele et al., Acta. Haematologica., 97:137-143, 1997) cytoplasmic reorganisation that megakaryocytes undergo to produce platelets, a process likened to the cytoplasmic blebbing observed in dying cells. Although the precise contribution various apoptotic regulators make to platelet biogenesis remains to be determined, it is concluded herein that Bcl-x_{L} is not absolutely required for megakaryocyte proliferation and differentiation. The role of apoptotic regulators in the process of platelet shedding is also addressed.

In a non-limiting embodiment, it is proposed herein that the amount of Bcl-x_{L} a platelet inherits determines its life span. As Bcl-x_{L} is degraded over time, a threshold is reached, upon which pro-apoptotic Bak is freed and platelet apoptosis is induced Inhibition of Bcl-x_{L}, either genetically or pharmacologically, speeds up the ‘molecular clock’, bringing forward the point of entry into cell death and subsequent platelet clearance from the circulation. The model is supported by the observation described herein that Bcl-x_{L}, and Bak have different half-lives: in the absence of new protein synthesis, Bcl-x_{L} degrades more rapidly than Bak. Given their limited synthetic capacity, it might therefore be expected that all else being equal, aging platelets would be unable to counter an inexorable decline in Bcl-x_{L} relative to Bak. Indeed, previous reports suggest that while Bak levels are stable in platelets stored at 37° C. (Brown et al., J. Biol. Chem., 275:5987-5996, 2000), Bcl-x_{L} levels decrease over time (Bertino et al., Transfusion (Paris), 45:857-866, 2003). A corollary is that older circulating platelets, harboring less Bcl-x_{L}, are more susceptible to the effects of ABT-737 than their younger counterparts, and our experiments with anti-platelet serum followed by ABT-737 treatment clearly demonstrate that this is the case. This probably explains near-normal recovery of platelet counts in mice treated daily with ABT-737, whereas mice that received the drug weekly exhibited acute-onset thrombocytopaenia interspersed with complete recovery between injections. In the face of sustained Bcl-x_{L} inhibition, the age profile of platelets presumably changes, such that the circulating population comprises primarily younger cells that are more refractory to ABT-737. With weekly injections, the age profile instead reverts to normal as the drug is cleared and the circulating platelet population is therefore normosensitive to ABT-737. Agents that antagonise Bcl-x_{L} in platelets cause thrombocytopaenia and which form a surrogate biomarker for Bcl-x_{L} inhibition in the clinic.

Thus, the present studies demonstrate that the intrinsic machinery for programmed cell death (apoptosis) regulates the life span of the anucleate platelet. Platelet survival, and hence life span, depends on pro-survival Bcl-x_{L} restraining the pro-apoptotic protein Bak and/or Bax. It has been reported previously that enucleated cytoplasts can undergo apoptosis (Jacobson et al., EMBO. J., 73:1899-1910, 1994). To our knowledge, platelets are the first example of an unmanipulated, anucleate cell that is not only capable of proceeding through programmed cell death, but whose life span is governed by the interplay between pro-survival and pro-apoptotic factors.

These studies indicate that mutations in the key genes controlling platelet survival account for some cases of inherited or acquired thrombocytopaenias and thrombocytoses. Strategies to promote platelet survival by inhibiting apoptosis will be advantageous in some patients with thrombocytopaenia. Conversely, patients suffering from thrombotic conditions or thrombocytosis will benefit from treatment with agents, that promote apoptosis such as BH3 mimetics like ABT-737, to promote platelet destruction and thus prevent the sequelae associated with uncontrolled clotting.

The present invention provides methods for the handling and storage of platelets prior to transfusion. Apoptotic processes have been implicated in the rapid decline in platelet viability observed ex vivo—the platelet storage lesion (Li et al., Transfusion (Paris), 40:1320-1329, 2000). Indeed, while Bcl-x_{L} levels decline in human platelets stored at 37° C., they do not appear to so in platelets subjected to routine blood bank storage procedures at 22° C. (Bertino et al., 2003 (supra)). In the light of our findings, maintaining the Bcl-x_{L}:Bak and/or Bax balance in favour of Bcl-x_{L} will be a key mechanism by which platelet viability is maintained during storage at room temperature and at the lower temperature. Thus, valuable improvements in platelet viability during storage and perhaps even post-transfusion, are contemplated by apoptosis inhibitory agents for stabilization of Bcl-x_{L} or inhibition of Bak and/or Bax. For example, agents inhibit apoptosis by promoting Bcl-x_{L} levels and/or activity may be contacted with platelets that have been collected for ex vivo storage and later administration to a subject in need thereof. Agents for promoting Bcl-x_{L} in favour of Bak and/or Bax may be added to platelets during harvesting of the platelets from a donor, for example by supplying the agent in the collection container. Alternatively, agents may be added during ex vivo storage of the platelets or during reinfusion of the platelets into the subject. Similarly, agents that antagonize Bak and/or Bax levels and/or activity may be contacted with platelets that are being stored ex vivo for later administration to a patient in need thereof. Apoptosis inhibitory agents may be contacted with the platelets to increase the half-life, viability or survival of the stored platelets that may be administered to a subject. For example, the agents may increase the half-life or survival of stored platelets by 10%, or greater, 20% or greater, 30% or greater, 40% or greater and 50% or greater. In another embodiment, agents may inhibit the activity of pro-apoptotic agents by 20% or greater, or 30% or greater, or 40% or greater or 50% or greater, or 60% or greater or 70% or greater, or 80% of greater, or 90% or greater.

Several groups have examined whether inhibition of caspases, the downstream demolition enzymes, can effectively delay storage-associated decreases in platelet viability. Even though reduction of enzyme activity was achieved, there was little impact on platelet viability (Bertino et al., 2003 (supra); Brown et al., 2000 (supra); Cohen et al., Thromb, Res., 113:387-393, 2004; Li et al., 2000 (supra)). This may reflect the short half lives of some inhibitors or their failure to completely abolish caspase activation, since low-level activity suffices for apoptosis (Methot, et al., J. Exp. Med., 199:199-207, 2004). More likely, even complete caspase inhibition may not prevent the organellar (particularly mitochondrial) damage directly induced by activated Bak (Green et al., 2004 (supra)). In one aspect, of this embodiment, inhibiting the apoptotic cascade at the level of the Bcl-x_{L}:Bak and/or Bax axis is proposed to overcome this important limitation.

### Example 12

### Platelet Viability and Functional Assays

Assays of platelet functions are described for example in Goncalves et al., J. Biol. Chem., 278(37):34812, 2003; Maxwell et al., J. Biol. Chem., 279(31):32196, 2004; Mangin et al., J. Biol. Chem., 278(35):32880, 2003; and Bakimer et al., J. Clin. Invest., 89(5): 1558, 1992. Of particular interest are the following assays.

### i) Standard Platelet Functional Assays

a) Platelet aggregation—This relatively simple technique involves stirring a suspension of platelets in the presence of a platelet activating substance and by monitoring changes in light transmission the device can accurately monitor platelet clumping (aggregation) in solution. This assay is useful at detecting changes in the adhesive function of the major platelet integrin α_{IIb}β₃ (GPIIb-IIIa).

b) Serotonin release assays—Platelet dense granules store various nucleotides (ADP and ATP) and other small molecule activators of platelets (serotonin, adrenaline and histamine). These molecules are released from platelets during thrombus development and play a major role in sustaining platelet activation. Monitoring of ¹⁴⁻C-serotonin release is a simple, reliable method of detecting defects in dense granule secretion,

c) Flow cytometry—Monitoring the surface expression of various markers of platelet activation, such as GPIIb-IIIa activation, P-selectin release and phosphatidylserine exposure, is important in defining specific abnormalities in platelet activation pathways. Defects in the expression of these markers are typically associated with abnormalities of platelet aggregation, α-granule secretion and procoagulant function, respectively,

d) Platelet adhesion assays—Allows the analysis of platelet adhesive interactions with various substrates, including fibrinogen and von Willebrand factor. In combination with imaging techniques such as Total Internal Reflection Fluorescence and epifluorescence microscopy, these assay systems allow simultaneous analysis of changes in cell morphology, receptor kinetics and near-membrane signal events. These assays are very useful at defining changes in membrane fluidity, spatio-temporal signalling events and cytoskeletal changes.

ii) In vitro Flow-Based Thrombosis Models

a) Platelet adhesion to purified thrombogenic proteins—These assays involve analysing adhesion of platelets onto purified von Willebrand factor, collagen or fibrinogen over a broad range of blood flow conditions. These simplified flow assays help define specific abnormalities in platelet adhesion responses and the likely receptors involved.

b) Platelet thrombus formation on thrombogenic surfaces—Involves perfusing native or anticoagulated whole blood on collagen or fibrinogen/vWf-coated surfaces and examining the 3-D dynamics of thrombus growth using confocal microscopy.

c) Real-time assessment of platelet adhesion and activation underflow—By employing calcium indicator dyes we can monitor simultaneous changes in calcium flux (sensitive marker of platelet activation) with changes in platelet morphology and adhesion dynamics. This is a powerful assay system to examine the temporal dynamics of platelet adhesion and activation.

d) Assessment of platelet thrombus formation in an ex-vivo vessel chamber—Involves perfusing native or anticoagulated whole blood through isolated vessel segments obtained from rodents or human tissue samples. Vascular injury is induced by FeCl3, through mechanical or photochemical means and platelet thrombus formation examining using various live cell imaging techniques. This is a useful technique to examine potential alterations in vascular reactivity to platelets and leukocytes, leading to an exaggerated thrombotic response.

iii) In vivo Thrombosis Models

a) Folts-type thrombosis model—The Folts model has principally been utilized in larger animals and is considered one of the gold standard experimental thrombosis models. This model has been adapted to the investigation of carotid artery thrombus formation in mice. The model involves repetitive crush injury to areas of arterial stenosis, resulting in platelet exposure to subendothelial thrombogenic components and high shear stress, two key factors promoting thrombus growth. Development of occlusive thrombi in mice typically occurred within 2-3 minutes of vascular injury and mechanical agitation of the vessel resulted in the embolisation of thrombi and restoration of normal blood flow. Constant formation and dislodgement of thrombi resulted in cyclic flow reductions (CFRs), a characteristic feature of the Folts model. The thrombi are characteristically platelet-rich and elimination of CFR is a hallmark feature of platelet dysfunction/inhibition.

b) Electrolytic model—The electrolytic thrombosis model has also been principally used in larger animals and leads to the development of platelet-rich thrombi in larger vessels. The model has been adapted to produce electrolytic injury to non-stenosed carotid arteries in mice. Electrolytic injury leads to full thickness vascular injury, triggering the formation of platelet-rich thrombi in mice (confirmed by histology) that ultimately occlude the artery within 15-20 minutes. Similar to the Folts model, this is an excellent system to assess defects in platelet function.

c) Laser injury model—A limitation of the Folts and electrolytic models is the inability to directly visualize the kinetics of thrombus growth. Intravital microscopy has been employed to visualize thrombus development in mouse mesenteric arterioles following laser-induced vascular injury. In this model, the mesentery is exteriorized and localized injury to the luminal surface of arterioles is induced by a pulsed nitrogen dye laser (440 nm; Micropoint Laser System, Photonics Instruments, St Charles, Ill.). This model allows graded vascular injury by adjusting the intensity of the laser. Platelet adhesion and aggregation and the overall kinetics of thrombus development can be visualized directly using an inverted Leica DMIRB microscope. This model is well characterized in terms of defining defects in platelet adhesive function.

### Example 13

### Assay of Mcl-1 Null MEF Cells

### 1. Introduction

Apoptosis is induced in Mcl-1^{(-/-)} cells by the compound ABT-737. The cells can be rescued from this effect by the general caspase inhibitor Q-VD-OPH. The assay aims to discover other compounds that have a comparable effect to that of Q-VD-OPH.

In summary, cells are split on day one in order to have them at a confluency of 60-80% on day two. On day two, the cells are seeded out into assay plates at a density that will ensure they are not confluent by day four of the assay. The assay plates are incubated at room temperature for 20-60 minutes before being transferred to 37° C. so that edge effects are minimized. For the same reason, assay plates are never stacked on top of each other in the incubator. On day three the cells are treated first with either Q-VD or with WEHI library compound. The cells are incubated for a 2 hour period in the presence of the library compounds and are then treated with ABT-737. Finally, on day four the cells are incubated for four hours in the presence of CellTitre-Blue™ Cell Viability Assay. This product contains resaruzin which is metabolized by live cells to resorufin. After four hours the level of resorufin is measured.

### 2. Reagents, Consumables and Instrumentation

Mel-1^{(-/-)} mouse embryonic fibroblasts (MEFs) were were grown in Iwaki 75 cm² tissue culture flasks (cat #3123-075). MEFs were grown in FMA media consisting of:
89% DME Kelso
10% heat-inactivated foetal calf serum (FCS) (Hyclone cat #SH30396.03)
1% 10 mM asparagine (Fluka cat. #11149)
275 μl of a 1:2000 dilution of 2-mercaptoethanol was added to the final 500 ml volume of FMA (Sigma cat #M7522; diluted in MT-PBS)

FMA was stored at 4° C. and used at 37° C.

MEFs were cultured and harvested using FMA media, MT-PBS (Parkville stores) and trypsin (Parkville stores). All reagents were stored at 4° C. and used at 37° C.

For assays, cells were seeded out in FMA media containing only 1% FCS. This consisted of:
98% DME Kelso (Parkville stores)
1% heat-inactivated foetal calf serum (FCS) (Hyclone cat #SH30396.03)
1% 10 mM asparagine (Fluka cat #11149)
275 μl of a 1:2000 dilution of 2-mercaptoethanol was added to the final 500 ml volume (Sigma cat #M7522; diluted in MT-PBS)

Assays were seeded out in Corning 384-well tissue culture grade black plates with flat, clear bottoms (DKSH Australia P/L cat #3712). Compounds were made up in Matrical 384-well 50 μl V-bottomed plates (cat #MP101-2-PP). Compound plates were sealed for overnight storage using foil seals from Beckman Coulter (cat #538619).

AnalaR grade DMSO was used for compound preparation and titrations (Merck cat #1.02952.2500). Trypan Blue Solution (0.4%) was used for cell counting (Sigma T8154). CellTitre-Blue™ Cell Viability Assay was sourced from Promega (cat #G8081), stored at −20° C. and used at 37° C. Q-VD-OPH general caspase inhibitor was used as a positive control (MP Biomedicals cat. #OPH109).

The Multidrop 384 (ThermoLabsystems) was used to seed the assay plates with cells and to add CellTitre-Blue™ viability reagent to cells. The Zymark Sciclone ALH3000 system was used for control and compound addition. The Wallac EnVision plate reader (Perkin Elmer) was used to measure fluorescence at λₑₓ 535 nm/λₑₘ 590 nm.

### 3. Method

3.1 Day One—Cell Splitting

Media was aspirated off the cells and they were then washed with 10 mls of warm MT-PBS. The PBS was aspirated off and 2 ml of trypsin was added to the flask. The flask was placed at 37° C. until the cells were detached. FMA media (˜6 ml) was used to wash the trypsin and cells to the bottom of the flask. The entire volume was transferred to a 50 ml centrifuge tube and centrifuged for 3 minutes at 250×g. The supernatant was aspirated off and the pellet resuspended in 4 ml of 10% PCS FMA. One millilitre of this cell suspension was added to a clean 75 cm² flask containing 19 ml of 10% FCS FMA media, thus performing a 1:4 split. This was repeated with the remaining cell suspension into other 75 cm² flasks, depending on the number of cells required for the following day's assay.

Assay plates for day two were labeled with barcodes.

3.2 Day Two—Seeding Assay Plates and Preparing Control Plates

The protocol for day one was repeated up to the point where the cells were pelleted and the supernatant aspirated off. The pellet was resuspended in 10 mls 1% FCS FMA. A 1:10 dilution was prepared in a 1.5 ml tube using 800 μl water, 100 μl cell suspension and 100 μl Trypan Blue Solution. The cells were vortexed and then counted using a haemocytometer. The dilution necessary to achieve a density of 2×10⁴ cells ml⁻¹ (1000 cells per well per 50 μl media) in the required volume was calculated and the dilution performed in 1% FCS FMA.

The Multidrop system was used to seed cells into all 384 wells of the assay plates. The system was set up to deliver 50 μl of cell suspension to each well. A sterile cassette head was used and rinsed thoroughly with sterile distilled water before use. The assay plates were rested at room temperature for 60 minutes and then placed at 37° C./5% CO₂ overnight. Plates were not stacked.

The Q-VD-OPH and ABT-737 plates were set up in Matrical compound plates. Q-VD was used at a stock concentration of 12.5 mM (final concentration in the cells of 25 μM). 10 μl of this stock was placed in wells I-P in columns 23 and 24. In all remaining wells, 10 μl of DMSO was dispensed. ABT-737 was at a stock concentration of 10 mM and was used at 10 μM in the compound plates (final concentration in the cells of 20 nM). Thus a 1:1000 dilution was performed and then 10 μl was dispensed into all wells of a 384-well Matrical plate except wells 23A-D, 24A-D, 23I-L and 24I-L. DMSO (10 μl) was dispensed into these 16 wells. Both plates were sealed with foil and stored overnight at 12° C.

3.3 Day Three—Treating the Cells

1. Library plates were removed from the freezer and allowed to thaw at room temperature for 30-60 minutes before use (note: the Q-VD-OPH addition takes ˜60 minutes so the plates can be thawing whilst the addition is occurring). It is important that the

2. The Zymark system was set up. The HEP A filter unit was turned on, the pintool was checked to ensure it was clean and unloaded and the deck was set up with blotting paper, ethanol and DMSO (refer to diagram 1).

3. The Q-VD-OPH was added to the cells. To do this, the Q-VD-OPH plate was placed on the deck (refer to diagram 1) with the A1 corner of the plate facing the corner of the room in which the EnVision computer sits. The assay plates were placed in stack 1 of the front Twister (refer to diagram 1).

Clara Execution Manager was opened on the Zymark desktop PC. All components were initialized by clicking on the “Initialize” button. Once initialization is completed, remove any old applications from the Applications Chain window and add the following ones in the stated order (for each application you need to enter the number of runs i.e. the number of assay plates you are treating):
1. ControlAddition
2. ControlAdditionRestack
3. PintoolUnload

Once these were in place and OKed the Material Initialization screen came up and was OKed. One of the Zymark Stack Storage system windows was then brought up and the configuration menu was accessed. “New” was chosen, then “ControlAddition” was chosen and OKed. The configuration menu was again accessed and the process repeated for the “ControlAdditionRestack” programme.

Once this was completed, the “Run” button on Clara was clicked to begin the run. At the end of the run the assay plates were left in the stacker and the Q-VD-OPH plate was removed from the Zymark deck.

4. The library compound addition was then begun. The compound plates were placed in stack 1 of the back Twister with A1 facing the MiniTrak (refer to diagram 1). Old applications were removed from the Applications Chain in Clara and the new ones were added in the following order with the number of runs being entered for each application:
1. PintoolAdditionCorning
2. PintoolUnload

Once this was done, it was OKed and the Material Initialization window was checked and OKed. One of the Zymark Stack Storage system windows was then brought up and the configuration menu was accessed, “New” was chosen, then “PintoolAdditionCorning” was chosen and OKed.

Once this was completed, the “Run” button on Clara was clicked to begin the run.

At the end of the run, the assay plates were re-lidded and returned to 37° C./5% CO₂ for the remainder of the 2 hours (timed from the compound addition to the first assay plate—generally around 30 minutes for a 20 plate run). The library plates were re-lidded and returned to freezer storage.

7. At the end of the 2 hour incubation the ABT-737 addition was carried out. The Q-VD plate was placed on the deck (refer to diagram 1) with the A1 corner of the plate facing the corner of the room in which the EnVision computer sits. The assay plates were placed in stack 1 of the front Twister.

Clara Execution Manager was opened on the Zymark desktop PC. All components were initialized by clicking on the “Initialize” button. Once initialization is completed, remove any old applications from the Applications Chain window and add the following ones in the stated order (for each application you need to enter the number of runs i.e. the number of assay plates you are treating):
1. ControlAddition
2. ControlAdditionRestack
3. PintoolUnload

Once these were in place and OKed the Material Initialization screen came up and was OKed. One of the Zymark Stack Storage system windows was then brought up and the configuration menu was accessed. “New” was chosen, then “ControlAddition” was chosen and OKed. The configuration menu was again accessed and the process repeated for the “ControlAdditionRestack” programme.

Once this was completed, the “Run” button on Clara was clicked to begin the run. At the end of the run the assay plates were re-lidded and returned to 37° C./5% CO₂ and the ABT-737 plate was removed from the Zymark deck.

8. The HEPA filter was turned off, the DMSO and ethanol reservoirs emptied and washed out and the pintool was cleaned following the protocol below:

Dip 10× in VP cleaning solution; sit for 5 minutes in VP cleaning solution; blot

Dip 10× in MQ water; blot

Dip 10× in 100% ethanol; blot

3.4 Day Four—Viability Analysis

CellTitre-Blue™ was warmed to 37° C. and 10 μl was then added to each well of the assay plates using the Multidrop. The plates were returned to 37° C. for 4 hours before being loaded into the EnVision plate reader. Viability measurements were taken and the data was then imported into Abase (IDBS) for analysis.

### Example 14

### Corticosteroids are Identified in the Subject Screen

A library of known compounds was screened using the protocol set out in Example 13. The results of preliminary analyses indicate several active molecules which were corticosteroids conforming to the following structural formula:

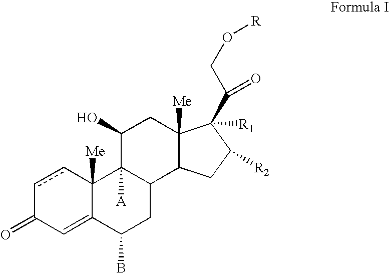

[symbol] can be either a single or double bond
A=H or F, B═H, CH₃, F or OH
R═H or C₂-C₆Acyl
R₁═H, OH or OC₂-C₆Acyl
R₂═H, Me or R₁ and R₂ form a dioxolane ring

In particular, these agents (see FIG. 10) were able to significantly inhibit killing of Mcl-1 null MEF cells by ABT-737. Accordingly, these agents are suitable for use in the present methods of enhancing or extending platelet viability life span or survival. Further, the agents find broad application in therapeutic interventions to extend or preserve life span of other cells.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
Summary of sequence identifiers

| SEQUENCE ID NO: | DESCRIPTION |
|---|---|
| 1 | nucleotide sequence cDNA mouse Bcl-x_{L} |
| 2 | amino acid sequence encoded by SEQ ID NO: 1 |
| 3 | nucleotide sequence cDNA mouse Bak |
| 4 | amino acid sequence encoded by SEQ ID NO: 3 |
| 5 | nucleotide sequence cDNA human Bcl-x_{L} |
| 6 | amino acid sequence encoded by SEQ ID NO: 5 |
| 7 | nucleotide sequence cDNA human Bak |
| 8 | amino acid sequence encoded by SEQ ID NO: 7 |
| 9 | nucleotide sequence cDNA human Bax |
| 10 | amino acid sequence encoded by SEQ ID NO: 9 |

TABLE 2
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Praline |
| influence chain | |
| orientation | |

TABLE 3
Exemplary and Preferred Amino Acid Substitutions

| Original | EXEMPLARY | PREFERRED |
|---|---|---|
| Residue | SUBSTITUTIONS | SUBSTITUTIONS |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

TABLE 4
Codes for non-conventional amino acids

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylasparatate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethy)glycine | Nthr |
| D-N-methylhistidine | Dnmhis | N-(hydroxyethy)glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mtrp | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

TABLE 5

| Additive | Blast | G | GM | M | Eₒ | Meg |
|---|---|---|---|---|---|---|
| Saline | 12 | 19 | 7 | 14 | 1 | 22 ± 3 |
| ABT-737 (1 μM) | 16 | 14 | 6 | 14 | 1 | 14 ± 6 |

TABLE 6
Peripheral blood cell values of mice carrying mutant alleles of Bcl-x

| | Bcl-x^{+/+} | Bcl-x^{+/Plt20} | Bcl-x^{+/Plt16} | Bcl-x^{Plt20/Plt20} | Bcl-x^{Plt16/Plt20‡} |
|---|---|---|---|---|---|
| Erythrocytes (×10⁶/μL) | 10.6 ± 0.3 | 10.7 ± 0.4 | 10.8 ± 0.5 | 10.2 ± 0.5 | 9.5 ± 0.4 |
| Hematocrit (%) | 51.5 ± 1.7 | 51.7 ± 2.3 | 52.4 ± 2.0 | 50.9 ± 1.6 | 49.6 ± 2.9 |
| MCV (fL) | 48.5 ± 0.7 | 48.5 ± 1.1 | 48.6 ± 1.0 | 50.2 ± 1.4 | 52.1 ± 1.6 |
| Leukocytes (×10³/μL) | 8.2 ± 1.5 | 8.2 ± 1.9 | 7.9 ± 1.7 | 8.4 ± 2.1 | 8.9 ± 0.8 |
| Neutrophils (×10³/μL) | 1.2 ± 0.2 | 1.2 ± 0.3 | 1.1 ± 0.2 | 1.4 ± 0.4 | 1.0 ± 0.3 |
| Lymphocytes (×10³/μL) | 7.1 ± 1.5 | 6.8 ± 1.7 | 6.7 ± 1.6 | 6.9 ± 1.6 | 7.3 ± 0.5 |
| Monocytes (×10³/μL) | 0.1 ± 0.0 | 0.1 ± 0.0 | 0.1 ± 0.0 | 0.1 ± 0.0 | 0.2 ± 0.0 |
| Platelets (×10³/μL) | 1,137 ± 82 | 598 ± 59 | 596 ± 65 | 265 ± 47 | 279 ± 48 |
| MPV (fL) | 7.1 ± 0.7 | 7.3 ± 0.7 | 6.7 ± 0.6 | 7.3 ± 0.6 | 7.3 ± 0.6 |

Values shown are mean ± 1 standard deviation
MCV, mean corpuscular volume; MPV, mean platelet volume
^{‡}All mice were on an inbred BALB/c background, with the exception of Bcl-x^{Plt16/Plt20} which was a mixture of C57BL/6 and BALB/c

### BIBLIOGRAPHY

Adams, Genes Dev., 17:2481-2495, 2003.
Altschul et al., Nucl. Acids Res. 25:3389, 1997.
Arkin et al., Proc. Natl. Acad. Sci. USA, 89:7811-7815, 1992.
Ault et al., Exp. Hematol., 23:996-1001, 1992.
Ausubel et al., Current Protocols in Molecular Biology John Wiley & Sons Inc, 1994-1998, Chapter 15
Ausubel (Ed) Current Protocols in Molecular Biology, 5^{th} Edition, John Wiley & Sons, Inc, NY, 2002.
Bacher et al., Drug Discovery Today, 3(6):265-273, 1998.
Baell, Biochem. Pharmacol., 64(5-6):851-863, 2002.
Bakimer et al., J. Clin. Invest., 89(5):1558, 1992.
Berger et al., Blood 92:4446-4452, 1998.
Bertino et al., Transfusion (Paris), 43:857-866, 2003.
Blajchman, Transfusion (Paris), 37:121-125, 1997.
Boise et al., Cell, 74:597-608, 1993.
Bonner et al., Eur. J. Biochem., 46:83, 1974.
Booyse et al., Biochim, Biophys. Acta., 757:660-663, 1968.
Bouillet et al., Science, 286:1735-1738, 1999.
Brecher et al., Transfusion (Paris), 43:974-978, 2003.
Brown et al., J. Biol. Chem., 275:5987-5996, 2000.
Carpinelli et al., Proc. Natl. Acad. Sci. U.S.A., 101:6553-6558, 2004.
Caserta et al., Apoptosis, 8:345-352, 2003.
Chen et al., Mol. Cell., 17:393-403, 2005.
Cheng et al., Mol. Cell., 8:705-711, 2001.
Chiu et al., Transfusion (Paris) 34:950-954, 1994.
Cohen et al., J. Med. Chem., 33:883-894, 1990.
Cohen et al., Thromb. Res., 113:387-393, 2004.
Coligan et al “Current Protocols in Immunology”, John Wiley & Sons, 1991.
Conner et al., Proc. Natl. Acad. Sci. USA, 80:278-282, 1983.
Cosulich et al., Current Biology, 7:913-920, 1997.
Culver, Gene Therapy: A Primer for Physicians, 2^{nd} Ed., Mary Ann Liebert, 1996.
Danial et al., Cell, 116:205-219, 2004.
Dayhoff et al., Natl. Biomed. Res. Found, 5:345-358, 1978.
De Botton et al., Blood 100:1310-1317, 2002.
Degterev et al., Nat. Cell. Biol, 5:173-182, 2001.
Delgrave et al., Protein Engineering, 6:327-331, 1993.
Denis et al., Cell, 222:379-391, 2005.
Diaz et al., J. Biol. Chem., 272:11350-11355, 1997.
Douillard et al., Basic Facts about Hybridomas, in Compendium of Immunology Vol. II, ed. by Schwartz, 1981.
Drachman, Blood, 103:390-398, 2004.
Enyedy et al., J. Med. Chem., 44:4313-4324, 2001.
Erickson et al., Science, 249:527-533, 1990.
Ekert et al., J. Cell. Biol., 165:835-842, 2004.
Fesik, Nat. Rev. Cancer, 5(11):876-885, 2005.
Finkelstein et al., Genomics, 7:167-172, 1990.
Fodor et al., Science, 251:767, 1991.
Franklin, Biochemistry, 42(27):8223-8231, 2003.
Friedman, In: Therapy for Genetic Disease, T. Friedman, Ed., Oxford University Press, pp. 105-121, 1991.
George et al., Ann. Intern. Med., 129:886-890, 1998.
Green et al., Science, 281:1309-1311, 1998.
Green et al., Science, 305:626-629, 2004.
Gold et al., Annu. Rev. Biochem., 64:763-797, 1995.
Goncalves et al., J. Biol. Chem., 278(37):34812, 2003.
Gonnet et al., Science, 256(5062):1443-1445, 1992.
Grillot et al., Journal of Experimental Medicine, 183:381-391, 1996.
Grompe et al., Proc. Nad. Acad. Sci. USA, 86:5855-5892, 1989.
Grompe, Proc. Natl. Acad. Sci. USA, 86:5855-5892, 1993.
Hacia et al., Nature Genetics, 14:441-447, 1996.
Hodgson, Biotechnology, 9:19-21, 1991.
Hogari et al., Manipulating the Mouse Embryo: A Laboratory Manual, Cold Spring Harbour Laboratory Press, CSH NY, 1986.
Holinger et al., J. Biol. Chem., 274:13298-13304, 1999.
Huang et al, EMBO. J., 16:4628-4638, 1997.
Huang et al, Cell, 103:839-842, 2000.
Jacobson et al., EMBO. J., 13:1899-1910, 1994.
Jayasena, Clin. Chem., 45(9):1628-1650, 1999.
Kaluzhny et al., Blood, 100:1670-1678, 2002.
Kasai et al., Dev Biol 264:202-216, 2003.
Kaufmann et al., Journal of Cell Biology, 160:53-64, 2003.
Kaushansky, Blood, 86:419-431, 1995.
Kieffer et al., Eur. J. Biochem., 164:189-195, 1987.
Kienast et al., Blood, 75:116-121, 1990.
Kile et al., Mamm. Genome, 14:81-85, 2003.
Kile et al., Nat. Rev. Genet., 6:557-567, 2005.
Kinszler et al., Science, 251:1366-1370, 1991.
Kitada et al., J. Med. Chem., 46:4259-4264, 2003.
Knudson et al., Science, 270:96-99, 1995.
Kohler et al., Nature, 256:495-499, 1975.
Kohler et al., European Journal of Immunology, 6:511-519, 1976.
Kothakota et al., Science, 278:294-298, 1997.
Kunkel, Proc. Natl. Acad. Sci. USA, 82:488-492, 1985.
Kunkel et al., Methods in Enzymol., 154:367-382, 1987.
Kurrek, Eur. J. Biochem., 270:1628-1644, 2003.
Kuter et al., Blood, 100:3457-3469, 2002.
Kyte et al., J. Mol. Biol., 157:105-132, 1982.
Leeksma et al., Nature, 175:552-553, 1955.
Li et al., Transfusion (Paris), 40:1320-1329, 2000.
Lindsten et al., Mol. Cell., 6:1389-1399, 2000.
Liu et al., Immunity, 19:341-352, 2003.
McDonnell et al., Cell, 57:79-88, 1989.
Mangin et al., J. Biol. Chem., 278(35):32880, 2003.
Mansour et al., Nature, 336:348-352, 1988.
Marmur et al., J. Mol. Biol., 5:109, 1962.
Marsden et al., Annu. Rev. Immunol., 21:71-105, 2003.
Maxwell et al., J. Biol. Chem., 279(31):32196, 2004.
Meng et al., J. Comp. Chem., 13:505-524, 1992.
Methot, et al., J. Exp. Med., 199:199-207, 2004.
Modrich, Ann. Rev. Genet., 25:229-253, 1991.
Morris et al., Proc. Natl. Acad. Sci., USA, 95(6):2902-2907, 1998.
Motoyama et al., Journal of Experimental Medicine, 189:1691-1698, 1999.
Motoyama et al, Science, 267:1506-1510, 1995.
Nakashima et al., Cancer Research, 60:1229-1235, 2000.
Navia et al., Current Opinions in Structural Biology, 2:202-210, 1992.
Newtown et al., Nucl. Acids. Res. 17:2503-2516, 1989.
O'Connor et al., EMBO. J., 17:384-395, 1998.
Ogilvy et al., Proc. Natl. Acad. Sci. U.S.A., 96:14943-14948, 1999.
Oltersdorf et al., Nature, 435:677-681, 2005.
Oost, J. Med. Chem., 47(18):4417-4426, 2004.
Orita et al., Proc. Nat. Acad. Sci. USA, 86:2776-2770, 1989.
Ottilie et al., Journal of Biological Chemistry, 272:30866-30872, 1997.
Peters et al., Physiol. Genomics., 11:185-193, 2002.
Pickert, Transgenic Animal Technology: A Laboratory Handbook, Academic Press, San Diago, Calif., 1994.
Print et al., Proc. Natl. Acad. Sci. USA, 95:12424-12431, 1998.
Rathmell et al., Nature Immunology, 3:932-939, 2002.
Rein et al., Nucleic Acids Research, 26(10):2255-2264, 1998.
Remington's Pharmaceutical Sciences, 18^{th} Ed., 1990, Mack Publishing, Company, Easton, Pa., U.S.A.
Riedl et al., Proceedings of the National Academy of Sciences of the USA, 98:14790-14795, 2001.
Roberg et al., Science, 269:202, 1995.
Ruano et al., Nucl. Acids. Res. 77:8392, 1989.
Rucker et al., Mol. Endocrinol., 14:1038-1052, 2000.
Sadowsky, J. Am. Chem. Soc., 127(34):11966-11968, 2005.
Sambrook, Molecular Cloning: A Laboratory Manual, 3^{rd} Edition, CSHLP, CSH, NY, 2001.
Sattler et al., Science, 275:983-986, 1997.
Schimmer et al., Cell Death Differ., 8:725-733, 2001.
Schimmer, Cell Death Differ., 13(2):179-188, 2006. Review.
Shangary, Biochemistry, 41:9485-9495, 2002.
Strasser et al., Proc Natl. Acad. Sci. U.S.A, 88:8661-8665, 1991.
Sheffield et al., Proc. Natl. Acad. Sci. USA, 86:232-236, 1989.
Sheffield et al., Am. J. Hum. Genet., 49:699-706, 1991.
Shoemaker et al. (Nature Genetics, 14:450-456, 1996.
Sumrnerton et al., Antisense and Nucleic acid Drug Development, 7:187-195, 1997.
Tavassoli, Blood, 55:537-545, 1980.
Thiele et al., Acta. Haematologica., 97:137-143, 1997.
Thornbeny et al., Science, 281:1312-1316, 1998.
Tzung et al., Nat Cell. Biol., 3:183-191, 2001.
van Delft et al., Cancer Cell, 10:389-399, 2006.
Veis et al., Cell, 75:229-240, 1993.
Vucic, Biochem. J., 385(Pt 1):11-20, 2005.
Walensky et al., Science, 305:1466-1470, 2004.
Wang et al., Proc. Natl. Acad. Sci. U.S.A., 97:7124-7129, 2000a.
Wang et al., Cancer Research, 60:1498-1502, 2000b.
Wang, J. Biol. Chem., 279(46):48168-48176, 2004.
Wartell et al., Nucl. Acids Res., 18:2699-2705, 1990.
Watson et al., “Molecular Biology of the Gene”, Fourth Edition, Benjamin/Cummings, Menlo Park, Calif., 1987.
Webb et al., Leuk. Lymphoma 34:71-84, 1999.
Wells, Methods Enzymol., 202:2699-2705, 1991.
Weyrich et al., Proc. Natl. Acad. Sci. USA, 95:5556-5561, 1998.
White et al., Genomics, 12:301-306, 1992.
White, Cell Death and Differentiation, 13:1371-1377, 2006.
Willis and Adams, Curr. Opin. Cell. Biol., 77:617-625, 2005.
Willis et al., Genes Dev., 19:1294-1305, 2005.
Wilson-Annan et al., J. Cell. Biol., 162:877-888, 2003.
Yang et al., Science, 275:1129-1132, 1997.
Yin, J. Am. Chem. Soc, 127(15):5463-5468, 2005.
Zong et al., Genes Dev., 15:1481-1486, 2001.

1. A method of enhancing or maintaining the viability or lifespan of platelets comprising administering to platelets an effective amount of an agent that down modulates apoptosis wherein the agent enhances the ratio of of Bcl-x_{L}:Bak in a cell, wherein the agent is an agonist of Bcl-x_{L} mediated apoptosis pathway or an antagonist of Bak, or Bax, or Bak and Bax.

2-7. (canceled)

8. The method of claim 1 wherein the agent is administered ex vivo or in vitro.

9. The method of claim 8 wherein the agent is administered to a blood product containing platelets.

10. The method of claim 9 wherein the blood product is whole blood or a platelet preparation.

11. The method of claim 1 wherein the agent is administered in vivo.

12. The method of claim 1 wherein the agent is administered to a subject suffering from or at risk of developing thrombocytopaenia.

13. The method of claim 12 wherein the subject is receiving chemotherapy.

14. The method of claim 11 comprising identifying a subject suffering from or at risk for thrombocytopaenia and administering the agent to the identified subject.

15-18. (canceled)

19. The method of claim 1 wherein the antagonist is a Bak-binding portion of Bcl-x_{L} or a variant or mimic thereof or a Bax-binding portion of Bcl-x_{L} or a variant or mimic thereof or a Bak and Bax-binding portion of Bcl-x_{L} or a variant or mimic thereof.

20. The method of claim 1 wherein the antagonist is a gene silencing agent.

21. The method of claim 1 wherein the agent is an antagonist of downstream effectors of Bak, or Bax, or Bak and Bax activity.

22. The method of claim 1 wherein the agent inhibits the uptake or cellular activity of apoptosis inducing agents in platelets.

23. A method of decreasing the survival, lifespan or viability of platelets comprising administering to platelets an effective amount of an agent that enhances apoptosis, wherein the agent is: an antagonist of Bcl-x_{L} mediated apoptosis pathway; an agonist of Bak polypeptide activity; an agonist of Bax polypeptide activity; an agonist of Bak and Bax polypeptide activity; or an IAP antagonist.

24-47. (canceled)

48. A method of screening for an agent which modulates the survival, lifespan or viability of platelets, said method comprising:
(i) contacting the agent with a system comprising a target selected from the group consisting of a Bcl-x_{L} and/or Bak or Bax polypeptide, and a Bcl-x, Bak or Bax genetic sequence; and
(ii) determining the presence of a complex between the agent and the target, a change in activity of the target, or a change in the level of activity of an indicator of the activity of the target.

49. A method of screening for a molecule which enhances the survival, lifespan or viability of platelets and/or other mammalian cells, said method comprising:
(iii) combining the molecule with a cell;
(iv) contacting the cell with one or more agents that antagonise pro-survival Bcl-2 family molecules in the cell and induce/s apoptosis;
(v) determining the change in survival (viability, lifespan, half-life) of cells in the presence of the molecule relative to a control;
(vi) selecting a molecule which enhances cell survival (viability, half-life); and
(vii) optionally combining the selected molecule from (iv) with platelets to determine the change in cell survival (viability, half-life) of platelets in the presence of the molecule relative to controls.

50. The method of claim 49 wherein the cell is modified to enhance its sensitivity to an apoptosis inducing agent.

51. The method of claim 50 wherein the cell is modified by reducing the level or activity of one or more pro-survival Bcl-2 family members.

52. The method of claim 50 where in the cell is modified to lack one or more pro-survival Bcl-2 family members by gene disruption.

53. The method of claim 49 wherein the cell is an Mcl-1 deficient cell from a multicellular organism and the agent is a Bcl-x_{L} antagonist.

54. The method of claim 49 further comprising: identifying modulation of a Bcl-2 family protein in the cell.

55. A modified population of platelets for administration to a subject in need thereof, the platelets comprising a population of platelets stored ex vivo and contacted with an apoptosis antagonist agent to increase platelet half-life.

56. The modified platelet population of claim 55 wherein the agent comprises an agonist of Bcl-x_{L} or an antagonist of Bak, Bax, or Bak and Bax.

57-64. (canceled)

65. The method of claim 1 wherein the agent is a small molecule

66. The method of claim 1, wherein the agent is an agent of Formula 1.

67. The method of claim 1, wherein the agent is selected from one of agents (a) to (d) in FIG. 10.

68-69. (canceled)


SEQUENCE LISTING (10 sequences)

SEQ ID NO: 1 - DNA, 2392 bp, mus musculus
gcgcttgcaagttcccccggtctcttcaggggaaactaaggccggcttctccgggagattcagcacgagcagtcagccagaaccttatcttggctttggatcctggaagagaatcgctaaacacagagcagacccagtaagtgagcaggtgttttggacaatggactggttgagcccatctctattataaaaatgtctcagagcaaccgggagctggtggtcgactttctctcctacaagctttcccagaaaggatacagctggagtcagtttagtgatgtcgaagagaataggactgaggccccagaagaaactgaagcagagagggagacccccagtgccatcaatggcaacccatcctggcacctggcggatagcccggccgtgaatggagccactggccacagcagcagtttggatgcgcgggaggtgattcccatggcagcagtgaagcaagcgctgagagaggcaggcgatgagtttgaactgcggtaccggagagcgttcagtgatctaacatcccagcttcacataaccccagggaccgcgtatcagagctttgagcaggtagtgaatgaactctttcgggatggagtaaactggggtcgcatcgtggcctttttctcctttggcggggcactgtgcgtggaaagcgtagacaaggagatgcaggtattggtgagtcggattgcaagttggatggccacctatctgaatgaccacctagagccttggatccaggagaacggcggctgggacacttttgtggatctctacgggaacaatgcagcagccgagagccggaaaggccaggagcgcttcaaccgctggttcctgacgggcatgactgtggctggtgtggttctgctgggctcactcttcagtcggaagtgaccagacactgaccgtccactcacctctcacctcccaccctgcccccaccacaactctctcttcagccaccattgctaccaggagaaccactacatgcaactcacgccccttgccctatcacagggttgggcctagacggagtcccctgcaattagctttctaggatctaccacacttctgtgaaagccaccttccccccacatctcagttctcttggcctcaaaactcacaaggttttcccctaaaccagctccttggaggctggcaggagcagagaggggtgtgtgagggaggagagcctgtcttgttggtgggaccctaattacccctgagcctctcgggaatgcttttccagcagggagctcttcccctgaaggagactagcttgccttggttttgatgtgtgtggcctcagacgtgatcatttcccatcccgctgtgtctctgggtctccctcctccccatctccaccccccaagagccattgagtgaggtgcttttagcccttttgactaattagaaattcaggctgcttgggataatgagacaaggaccagaacctcctccccacctgtggcctggccaaaacccccacttgtggtctgaatgttctcctgaggcctctggctagagtctagccccacccagggggaggggcttagctgctgaaagtccaccttgctagagctagggtggctcttgcagcttagcaccccccaagatccttgtccacccctcccccggctcccatgaccatgactgagggaccaatgggcccaagataggtgccccagagctgttaatgacttcagctgcctcacttcctgcaagattggcctgtggcatctttgccttgggtgttggccacagggtcaagggactctggcctcagcccagaagtgaggggaagctatacagagcagctgtgggaaccccaggggcttccctacctcaggcaggaagggcaggaagaagagcctggagcccaggcgagggggacggcaagggcctgtctttgtgctctgccccacctagcctgggcagcagggctgccaaggtcagaatggcctggctaggagcccttcaggcctctctctcctctgtccacccttggcctgtctcatccctgtgggtcccagctcagctctggccaccccgggctctctgctgtacatattcgagactagtttttattccttgtgaagatgatatactatttttgttaagcgtgtctgtatttatgtgtgaggagctgctggcttgctgtgcgcgtgcccgtggagagctggtgcccagagactgacagcctgatgctccctccctgcccttgcctggggaagcccaccaggggttctggcttctgaggggcacctgtccctcctctcccctcaccctacacttgttccagctctttgaaatagtttgtgtgaaggtgaaagtgcagttcagtaataaatggtgtctgtgactctgtgaaaaaaaaaaaaaaaaaaaaaaa

SEQ ID NO: 2 - protein, 233 aa, mus musculus
MSQSNRELVVDFLSYKLSQKGYSWSQFSDVEENRTEAPEETEAERETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIASWMATYLNDHLEPWIQENGGWDTFVDLYGNNAAAESRKGQERFNRWFLTGMTVAGVVLLGSLFSRK

SEQ ID NO: 3 - DNA, 831 bp, mus musculus
cggacgcgtgggcggacgcgtgggcgccgcagccgccggggcgcacccggcgagaggcagcggcagtgatggacgggtccggggagcagcttgggagcggcgggcccaccagctctgaacagatcatgaagacaggggcctttttgctacagggtttcatccaggatcgagcagggaggatggctggggagacacctgagctgaccttggagcagccgccccaggatgcgtccaccaagaagctgagcgagtgtctccggcgaattggagatgaactggacagcaatatggagctgcagaggatgattgctgacgtggacacggactccccccgagaggtcttcttccgggtggcagctgacatgtttgctgatggcaacttcaactggggccgcgtggttgccctcttctactttgctagcaaactggtgctcaaggccctgtgcactaaagtgcccgagctgatcagaaccatcatgggctggacactggacttcctccgtgagcggctgcttgtctggatccaagaccagggtggctgggaaggcctcctctcctacttcgggacccccacatggcagacagtgaccatctttgtggctggagtcctcaccgcctcgctcaccatctggaagaagatgggctgaggcctcccactgccttggactgtgtcttttcttcataaattatgacattttcctgggatgaatgggggaaggggaaaggcatttttcttacttttgtaattattgggaggggtgggaatggtggcctggggaggcgccaataaacctcaggtcccctttgaaaaaaaaaaaaaaaaaaaaaaaa

SEQ ID NO: 4 - protein, 192 aa, mus musculus
MDGSGEQLGSGGPTSSEQIMKTGAFLLQGFIQDRAGRMAGETPELTLEQPPQDASTKKLSECLRRIGDELDSNMELQRMIADVDTDSPREVFFRVAADMFADGNFNWGRVVALFYFASKLVLKALCTKVPELIRTIMGWTLDFLRERLLVWIQDQGGWEGLLSYFGTPTWQTVTIFVAGVLTASLTIWKKMG

SEQ ID NO: 5 - DNA, 2575 bp, homo sapien
ggaggaggaagcaagcgagggggctggttcctgagcttcgcaattcctgtgtcgccttctgggctcccagcctgccgggtcgcatgatccctccggccggagctggtttttttgccagccaccgcgaggccggctgagttaccggcatccccgcagccacctcctctcccgacctgtgatacaaaagatcttccgggggctgcacctgcctgcctttgcctaaggcggatttgaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaaccactacatgcagcccatgcccacctgcccatcacagggttgggcccagatctggtcccttgcagctagttttctagaatttatcacacttctgtgagacccccacacctcagttcccttggcctcagaattcacaaaatttccacaaaatctgtccaaaggaggctggcaggtatggaagggtttgtggctgggggcaggagggccctacctgattggtgcaacccttaccccttagcctccctgaaaatgtttttctgccagggagcttgaaagttttcagaacctcttccccagaaaggagactagattgcctttgttttgatgtttgtggcctcagaattgatcattttccccccactctccccacactaacctgggttccctttccttccatccctaccccctaagagccatttaggggccacttttgactagggattcaggctgcttgggataaagatgcaaggaccaggactccctcctcacctctggactggctagagtcctcactcccagtccaaatgtcctccagaagcctctggctagaggccagccccacccaggagggagggggctatagctacaggaagcaccccatgccaaagctagggtggcccttgcagttcagcaccaccctagtcccttcccctccctggctcccatgaccatactgagggaccaactgggcccaagacagatgccccagagctgtttatggcctcagctgcctcacttcctacaagagcagcctgtggcatctttgccttgggctgctcctcatggtgggttcaggggactcagccctgaggtgaaagggagctatcaggaacagctatgggagccccagggtcttccctacctcaggcaggaagggcaggaaggagagcctgctgcatggggtggggtagggctgactagaagggccagtcctgcctggccaggcagatctgtgccccatgcctgtccagcctgggcagccaggctgccaaggccagagtggcctggccaggagctcttcaggcctccctctctcttctgctccacccttggcctgtctcatccccaggggtcccagccaccccgggctctctgctgtacatatttgagactagtttttattccttgtgaagatgatatactatttttgttaagcgtgtctgtatttatgtgtgaggagctgctggcttgcagtgcgcgtgcacgtggagagctggtgcccggagattggacggcctgatgctccctcccctgccctggtccagggaagctggccgagggtcctggctcctgaggggcatctgcccctcccccaacccccaccccacacttgttccagctctttgaaatagtctgtgtgaaggtgaaagtgcagttcagtaataaactgtgtttactcagtgaaaaaaaaaaaaaaaaaa

SEQ ID NO: 6 - protein, 233 aa, homo sapien
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 7 - DNA, 2203 bp, homo sapien
gtctgcatccggtggccacagagcaacttcctctagagggagctgattggagccgggtgccgctggcacctctatgatcactggagtctcgcgggtccctcgggctgcacagggacaagtaaaggctacatccagatgccgggaatgcactgacgcccattcctggaaactgggctcccactcagcccctgggagcagcagccgccagcccctcgggacctccatctccaccctgctgagccacccgggttgggccaggatcccggcaggctgatcccgtcctccactgagacctgaaaaatggcttcggggcaaggcccaggtcctcccaggcaggagtgcggagagcctgccctgccctctgcttctgaggagcaggtagcccaggacacagaggaggttttccgcagctacgttttttaccgccatcagcaggaacaggaggctgaaggggtggctgcccctgccgacccagagatggtcaccttacctctgcaacctagcagcaccatggggcaggtgggacggcagctcgccatcatcggggacgacatcaaccgacgctatgactcagagttccagaccatgttgcagcacctgcagcccacggcagagaatgcctatgagtacttcaccaagattgccaccagcctgtttgagagtggcatcaattggggccgtgtggtggctcttctgggcttcggctaccgtctggccctacacgtctaccagcatggcctgactggcttcctaggccaggtgacccgcttcgtggtcgacttcatgctgcatcactgcattgcccggtggattgcacagaggggtggctgggtggcagccctgaacttgggcaatggtcccatcctgaacgtgctggtggttctgggtgtggttctgttgggccagtttgtggtacgaagattcttcaaatcatgactcccaagggtgccctttggggtcccggttcagacccctgcctggacttaagcgaagtctttgccttctctgttcccttgcaggggtcccccctcaagagtacagaagctttagcaagtgtgcactccagcttcggagggcccctgcgtgggggccagtcaggctgcagaggcacctcaacattgcatggtgctagtgggccctctctctgggcccaggggctgtggccgtctcctccctcagctctctgggacctccttagccctgtctgctaggcgctggggagactgataacttggggaggcaagagactgggagccacttctccccagaaagtgtttaacggttttagctttttataatacccttgtgagagcccattcccaccattctacctgaggccaggacgtctggggtgtggggattggtgggtctatgttccccaggattcagctattctggaagatcagcaccctaagagatgggactaggacctgagcctggtcctggccgtccctaagcatgtgtcccaggagcaggacctactaggagaggggggccaaggtcctgctcaactctacccctgctcccattcctccctccggccatactgcctttgcagttggactctcagggattctgggcttggggtgtggggtggggtggagtcgcagaccagagctgtctgaactcacgtgtcagaagcctccaagcctgcctcccaaggtcctctcagttctctcccttcctctctccttatagacacttgctcccaacccattcactacaggtgaaggctctcacccccatccctgggggccttgggtgagtggcctgctaaggctcctccttgcccagactacagggcttaggacttggtttgttatatcagggaaaaggagtagggagttcatctggagggttctaagtgggagaaggactatcaacaccactaggaatcccagaggtgggatcctccctcatggctctggcacagtgtaatccaggggtgtagatgggggaactgtgaatacttgaactctgttcccccaccctccatgctcctcacctgtctaggtctcctcagggtggggggtgacagtgccttctctattgggcacagcctagggtcttgggggtcaggggggagaagttcttgattcagccaaatgcagggaggggaggcagatggagcccataggccaccccctatcctctgagtgtttggaaataaactgtgcaatcccctcaccctgaaaaaaaaaaa

SEQ ID NO: 8 - protein, 211 aa, homo sapien
MASGQGPGPPRQECGEPALPSASEEQVAQDTEEVFRSYVFYRHQQEQEAEGVAAPADPEMVTLPLQPSSTMGQVGRQLAIIGDDINRRYDSEFQTMLQHLQPTAENAYEYFTKIATSLFESGINWGRVVALLGFGYRLALHVYQHGLTGFLGQVTRFVVDFMLHHCIARWIAQRGGWVAALNLGNGPILNVLVVLGVVLLGQFVVRRFFKS

SEQ ID NO: 9 - DNA, 579 bp, homo sapien
atggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcgctcaccatctggaagaagatgggctga

SEQ ID NO: 10 - protein, 192 aa, homo sapien
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG